(19) United States
(12) Patent Application Publication
(10) Pub. No.: US 2019/0072555 A1
(43) Pub. Date: Mar. 7, 2019

(54) MULTIPLEX ASSAYS FOR EVALUATING PROSTATE CANCER STATUS

(71) Applicant: OPKO Diagnostics, LLC, Woburn, MA (US)

(72) Inventor: Vincent Linder, Tewksbury, MA (US)

(73) Assignee: OPKO Diagnostics, LLC, Woburn, MA (US)

(21) Appl. No.: 16/053,849

(22) Filed: Aug. 3, 2018

Related U.S. Application Data
(60) Provisional application No. 62/545,288, filed on Aug. 14, 2017.

(51) Int. Cl. G01N 33/574; G01N 33/68; G06F 19/12; G01N 33/53; B01L 3/00

(52) U.S. Cl. CPC G01N 33/57434; G01N 33/689; G06F 19/12; B01L 2300/0819; B01L 3/502753; G01N 2333/811; G01N 33/5304

(57) ABSTRACT

Aspects of the disclosure relate to improved assay systems for measuring certain markers (e.g., kallikrein proteins) and determining their presence and/or level in a sample. Further, aspects of the disclosure related to improved methods are provided for determining the probability of an event associated with prostate cancer in a subject.

### RELATED APPLICATIONS

This application claims the benefit of U.S. Provisional Patent Application Ser. No. 62/545,288, filed Aug. 14, 2017 entitled “MULTIPLEX ASSAYS FOR EVALUATING PROSTATE CANCER STATUS”, which is incorporated herein by reference in its entirety for all purposes.

### FIELD OF THE INVENTION

This disclosure relates to apparatuses and methods for predicting risk of prostate cancer. More particularly this disclosure relates to solid phase assay systems and methods of use therefore in the prediction of risk of prostate cancer.

### BACKGROUND OF INVENTION

Elevated blood levels of total prostate-specific antigen (PSA) are associated with prostate-related disorders, including prostate cancer. There is evidence that measuring levels of multiple different prostate cancers proteins (markers) can lead to improved predictions relating to the presence of prostate cancer in a subject. However, there remains a need for improved assay devices and methods for assessing prostate cancer.

### SUMMARY OF INVENTION

Aspects of the disclosure relate to improved assay systems for measuring certain proteins (markers) (e.g., kallikrein proteins) and determining their presence and/or level in a sample. Multiplex assays detecting and/or accurately measuring the amount of different proteins are challenging, and become even more challenging when the proteins being measured are highly similar (e.g., have high sequence similarity). Highly related proteins may share one or more epitopes (i.e., have common epitopes) and may be recognized by the same antibodies. However, in certain circumstances it may be advantageous to determine the levels of each protein in spite of the presence of one or more highly related proteins in a sample. Accordingly, this application provides articles and methods for overcoming the difficulties associated with evaluating multiple different antigens and uses thereof for measuring the amounts of proteins with common epitopes.

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein the one or more binding partners in the first liquid containment region each specifically bind to an epitope present on one or more proteins selected from the group consisting of: total prostate specific antigen (tPSA), free prostate specific antigen (fPSA), and free human kallikrein 2 (fhK2); and wherein the one or more binding partners in the second liquid containment region each specifically bind to an epitope present on one or more proteins selected from the group consisting of: intact prostate specific antigen (iPSA), macrophage inhibitory cytokine-1 (MIC-1), and beta-microseminoprotein (MSMB); and a detector configured to measure a signal indicative of the extent of binding of the one or more binding partners to the one or more epitopes. According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein the one or more binding partners in the first liquid containment region each specifically bind to an epitope present on one or more proteins selected from the group consisting of: total prostate specific antigen (tPSA), free prostate specific antigen (fPSA), and total human kallikrein 2 (thK2); and wherein the one or more binding partners in the second liquid containment region each specifically bind to an epitope present on one or more proteins selected from the group consisting of: intact prostate specific antigen (iPSA), macrophage inhibitory cytokine-1 (MIC-1), and beta-microseminoprotein (MSMB); and a detector configured to measure a signal indicative of the extent of binding of the one or more binding partners to the one or more epitopes.

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein the one or more binding partners in the first liquid containment region each specifically bind to an epitope present on one or more proteins selected from the group consisting of: total prostate specific antigen (tPSA), free prostate specific antigen (fPSA), and total human kallikrein 2 (thK2); and wherein the one or more binding partners in the second liquid containment region each specifically bind to an epitope present on one or more proteins selected from the group consisting of: macrophage inhibitory cytokine-1 (MIC-1), and beta-microseminoprotein (MSMB); and a detector configured to measure a signal indicative of the extent of binding of the one or more binding partners to the one or more epitopes.

In some embodiments, the one or more binding partners in the second liquid containment region each specifically bind to an epitope present on one or more proteins selected from the group consisting of: intact prostate specific antigen (iPSA), macrophage inhibitory cytokine-1 (MIC-1), and beta-microseminoprotein (MSMB). In another embodiment, the one or more binding partners in a second liquid containment region each specifically bind to an epitope present on one or more proteins selected from the group consisting of: macrophage inhibitory cytokine-1 (MIC-1) and beta-microseminoprotein (MSMB).

According to some aspects of the disclosure, provided herein is a solid-phase assay system comprising: a chip comprising a substantially rigid substrate and one or more fluidically isolated liquid containment regions, each liquid containment region having one or more analysis regions, wherein each analysis region comprises one or more binding partners immobilized to a substrate portion therein, wherein a first binding partner in a first liquid containment region binds to an epitope present on free prostate specific antigen (fPSA); and wherein the first liquid containment region or a second liquid containment region further comprises one or more binding partners that each bind to an epitope present on a protein selected from the group: fhK2, thK2, MIC-1, MSMB, and tPSA; and a detector configured to measure a signal indicative of the extent of binding of the one or more binding partners to the one or more epitopes.

In some embodiments, the solid-phase assay system further comprises a binding partner in the second liquid containment region binds to an epitope present on intact prostate specific antigen (iPSA).

According to some aspects of the disclosure, provided herein is a solid-phase assay system comprising: a chip comprising a substantially rigid substrate and one or more fluidically isolated liquid containment regions, each liquid containment region comprising one or more analysis regions, wherein each analysis region comprises one or more binding partners immobilized to a substrate portion therein, wherein a first binding partner in the first liquid containment region binds to an epitope present on free human kallikrein 2 (fhK2); and a detector configured to measure a signal indicative of the extent of binding of the one or more binding partners to the one or more epitopes.

According to some aspects of the disclosure, provided herein is a solid-phase assay system comprising: a chip comprising a substantially rigid substrate and two or more fluidically isolated liquid containment regions, each liquid containment region having one or more analysis regions, wherein each analysis region comprises one or more binding partners immobilized to a substrate portion therein, wherein a first binding partner in the first liquid containment region binds to an epitope present on free prostate specific antigen (fPSA); and wherein a second binding partner in the second liquid containment region binds to an epitope present on intact prostate specific antigen (iPSA); and a detector configured to measure a signal indicative of the extent of binding of the one or more binding partners to the one or more epitopes.

In some embodiments, the first liquid containment region or the second liquid containment region further comprises one or more binding partners that each bind to an epitope present on a protein selected from the group: fhK2, thK2, MIC-1, MSMB, and tPSA.

According to some aspects of the disclosure, provided herein is a solid-phase assay system comprising: a chip comprising a substantially rigid substrate and one or more fluidically isolated liquid containment regions, each liquid containment region comprising one or more analysis regions, wherein each analysis region comprises one or more binding partners immobilized to a substrate portion therein, wherein a first binding partner in a first liquid containment region binds to an epitope present on total human kallikrein 2 (thK2); and wherein the first liquid containment or a second liquid containment region further comprises one or more binding partners that each bind to an epitope present on a protein selected from the group: MIC-1, MSMB, tPSA, and fPSA; and a detector configured to measure a signal indicative of the extent of binding of the one or more binding partners to the one or more epitopes.

In one embodiment, the first liquid containment region or the second liquid containment region further comprises a binding partner that binds to an epitope present on a protein selected from the group: tPSA and fPSA; and wherein the first binding partner has about a 20-fold higher affinity (e.g., an 18-fold, 19-fold, 20-fold, 21-fold, 22-fold, 23-fold, 24-fold, 25-fold, 26-fold, 27-fold, 28-fold, 29-fold, 30-fold, or higher affinity) for hK2 than for tPSA and/or fPSA.

In some embodiments, the second liquid containment region further comprises a binding partner that binds to an epitope present on a protein selected from the group: tPSA and fPSA; and wherein the first binding partner and the second binding partner have approximately equal affinities (e.g., about 0.7-fold, about 0.8-fold. about 0.9-fold, about 1-fold, about 1.1-fold, about 1.2-fold, about 1.3-fold, about 1.4-fold, about 1.5-fold, about 1.6-fold, about 1.7-fold, about 1.8-fold, about 1.9-fold, or 2-fold) for hK2. In some embodiments, the first liquid containment or the second liquid containment region further comprises two or more binding partners that each bind to an epitope present on a protein selected from the group: MIC-1, MSMB, tPSA, and fPSA. In certain embodiments, the first or second liquid containment region further comprises one or more binding partners that bind to an epitope present on intact prostate specific antigen (iPSA).

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein a first binding partner in the first liquid containment region binds to an epitope present on free human kallikrein 2 (fhK2); and wherein a second binding partner in the second liquid containment region binds to an epitope present on intact prostate specific antigen (iPSA); and a detector configured to measure a signal indicative of the extent of binding of the one or more binding partners to the one or more epitopes.

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein a first binding partner in the first liquid containment region binds to an epitope present on total human kallikrein 2 (thK2); and wherein a second binding partner in the second liquid containment region binds to an epitope present on intact prostate specific antigen (iPSA); and a detector configured to measure a signal indicative of the extent of binding of the one or more binding partners to the one or more epitopes.

In some embodiments, the first liquid containment or the second liquid containment region further comprises one or more binding partners that each bind to an epitope present on a protein selected from the group: MIC-1, MSMB, tPSA, and fPSA. In some embodiments, the first liquid containment or the second liquid containment region further comprises two or more binding partners that each bind to an epitope present on a protein selected from the group: MIC-1, MSMB, tPSA, and fPSA.

In some embodiments, the solid-phase assay system further comprises one or more additional liquid containment regions. In certain embodiments, the solid-phase assay system further comprises a third liquid containment region. In some embodiments, the third liquid containment region further comprises one or more binding partners that each bind to a protein selected from the group: MIC-1, MSMB, tPSA, and fPSA. In some embodiments, the third liquid containment region further comprises two or more binding partners that each bind to a protein selected from the group: MIC-1, MSMB, tPSA, and fPSA. In certain embodiments, the third liquid containment region further comprises a binding partner that binds to MIC-1 and a binding partner that binds MSMB. In some embodiments, the third liquid containment region further comprises a binding partner that binds to iPSA.

According to some aspects of the disclosure, provided herein is a solid-phase assay system comprising: a chip comprising a substantially rigid substrate and one or more fluidically isolated liquid containment regions, each liquid containment region comprising multiple binding partners immobilized to distinct substrate portions within one or more analysis regions, wherein each of the multiple binding partners in a first liquid containment region specifically binds to a unique epitope on a protein selected from the group consisting of: total prostate specific antigen (tPSA), free prostate specific antigen (fPSA), and free human kallikrein 2 (fhK2); and a detector configured to measure a signal indicative of the extent of binding of the multiple binding partners to the unique epitopes.

In one embodiment, the multiple binding partners in a second liquid containment region each specifically bind to a unique epitope present on one or more proteins selected from the group consisting of: intact prostate specific antigen (iPSA), macrophage inhibitory cytokine-1 (MIC-1), and beta-microseminoprotein (MSMB). In another embodiment, the multiple binding partners in a second liquid containment region each specifically bind to a unique epitope present on one or more proteins selected from the group consisting of: macrophage inhibitory cytokine-1 (MIC-1) and beta-microseminoprotein (MSMB).

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein the one or more binding partners in the first liquid containment region each specifically bind to an epitope present on one or more proteins selected from the group consisting of: total prostate specific antigen (tPSA), free prostate specific antigen (fPSA), and free human kallikrein 2 (fhK2); and wherein the one or more binding partners in the second liquid containment region each specifically bind to an epitope present on one or more proteins selected from the group consisting of: macrophage inhibitory cytokine-1 (MIC-1), and beta-microseminoprotein (MSMB); and a detector configured to measure a signal indicative of the extent of binding of the one or more binding partners to the one or more epitopes. According to some aspects of the disclosure, provided herein is a solid-phase assay system comprising: a chip comprising a substantially rigid substrate and one or more fluidically isolated liquid containment regions, each liquid containment region comprising multiple binding partners immobilized to distinct substrate portions within one or more analysis regions, wherein each of the multiple binding partners in a first liquid containment region specifically bind to an epitope present on one or more proteins selected from the group consisting of: total prostate specific antigen (tPSA), free prostate specific antigen (fPSA), and total human kallikrein 2 (thK2); and a detector configured to measure a signal indicative of the extent of binding of the one or more binding partners to the one or more epitopes. In one embodiment, the multiple binding partners in the second liquid containment region each specifically bind to a unique epitope present on one or more proteins selected from the group consisting of: intact prostate specific antigen (iPSA), macrophage inhibitory cytokine-1 (MIC-1), and beta-microseminoprotein (MSMB)

According to some aspects of the disclosure, provided herein is a solid-phase assay system comprising: a chip comprising a substantially rigid substrate and two or more fluidically isolated liquid containment regions, each liquid containment region comprising one or more analysis regions, wherein each analysis region comprises one or more binding partners immobilized to a substrate portion therein, wherein a first binding partner in the first liquid containment region binds to an epitope present on a first kallikrein protein, wherein a second binding partner in the second liquid containment region binds to an epitope present on a second kallikrein protein, wherein the first kallikrein protein and the second kallikrein protein have at least one common epitope; and a detector configured to measure a signal indicative of the extent of binding of the one or more binding partners to the one or more epitopes.

In some embodiments, the first kallikrein protein and the second kallikrein protein are each selected from the group: free human kallikrein 2 (fhK2), total human kallikrein 2 (thK2), total prostate specific antigen (tPSA), and free prostate specific antigen (fPSA). In some embodiments, the first kallikrein protein and the second kallikrein protein are each selected from the group: free human kallikrein 2 (fhK2), total human kallikrein 2 (thK2), total prostate specific antigen (tPSA), intact prostate specific antigen (iPSA), and free prostate specific antigen (fPSA). In certain embodiments, the first kallikrein protein is fPSA and the second kallikrein protein is iPSA. In some embodiments, the first kallikrein protein is fPSA and the second kallikrein protein is fhK2 or thK2. In some embodiments, the first kallikrein protein is selected from fhK2 or thK2 and the second kallikrein protein is iPSA. In some embodiments, the first kallikrein protein is tPSA and the second kallikrein protein is fhK2 or thK2.

According to some aspects of the disclosure, provided herein is an in vitro method, comprising: (a) introducing a sample from a subject into an assay system, wherein the assay system comprises the chip of any preceding claim; (b) allowing one or more epitopes from the sample to bind with the one or more binding partners in the one or more analysis regions; (c) determining a characteristic of one or more proteins using one or more detectors associated with the one or more analysis regions; (d) inputting the characteristics of the one or more proteins into a processor programmed to evaluate a logistic regression model to determine the probability of an event associated with prostate cancer in a subject, wherein evaluating the logistic regression model comprises scaling each of a plurality of variables by a different coefficient value to produce scaled variables and summing values for the scaled variables used to produce the probability of the event associated with prostate cancer in a subject, wherein the plurality of variables includes one or more clinical variables and two or more variables included in the information received from the detector; and (e) determining the probability of the event associated with prostate cancer in the subject.

In some embodiments, the method additionally comprises obtaining the sample from the subject. In some embodiments, the characteristic of the one or more proteins is a signal indicative of the extent of binding of the one or more binding partners to the one or more epitopes. In some embodiments, the clinical variables are selected from the group consisting of: age, family history, and previous prostate biopsy.

In certain embodiments, the plurality of variables further comprises one or more of the following variables: prostate volume, abnormal or suspicious results from a digital rectal examination, and the presence or absence or one or more single nucleotide polymorphisms (SNPs).

In some embodiments, the sample is serum, plasma, or whole blood. In some embodiments, the subject is a human.

In some embodiments, the one or more SNPs are selected from: rs138213197, rs7818556, rs6983267, rs10993994, rs12793759, rs16901979, rs9911515, rs1016343, rs7106762, rs6579002, rs16860513, rs5945619, rs16902094, rs10896437, rs651164, rs7679673, rs13265330, rs2047408, rs10107982, rs620861, rs9297746, rs1992833, rs7213769, rs2710647, rs888507, rs17021918, rs12500426, rs2028900, rs7102758, rs16901922, rs6062509, rs2659051, rs17832285, rs12543663, rs4699312, rs11091768, rs3120137, rs6794467, rs10086908, rs7141529, rs2315654, rs12151618, rs747745, rs1009, rs2132276, rs2735839, rs11568818, rs684232, rs9364554, rs9830294, rs2660753, rs10807843, rs1933488, rs17467139, rs12947919, rs721048, rs385894, rs2331780, rs1894292, rs2107131, rs6545962, rs11649743, rs758643, rs2297434, rs902774, rs2647262, rs17224342, rs5918762, rs11672691, rs17138478, rs3019779, rs1873555, rs9457937, rs2838053, rs12946864, rs12475433, rs3765065, rs2018334, rs3771570, rs4871779, rs10875943, rs11601037, rs6489721, rs11168936, rs9297756, rs11900952, rs6569371, rs7752029, rs5934705, rs3745233, rs1482679, rs749264, rs6625760, rs5978944, rs2366711, rs5935063, rs10199796, rs2473057, rs4925094, rs3096702, rs12490248, rs4245739, rs10094059, rs306801, rs2823118, rs2025645, rs9359428, rs10178804, rs6090461, rs2270785, rs16901841, rs2465796, rs17256058, rs16849146, rs2269640, rs8044335, rs6530238, rs712242, rs9267911, rs11134144, rs12880777, rs7090755, rs132774, rs17779822, rs398146, rs4844228, rs4237185, rs7125415, rs1439024, rs6770955, rs11253002, rs4822763, rs2162185, rs12640320, rs5945637, rs3818714, rs6762443, rs10508678, rs2272668, rs2227270, rs6437715, rs3759129, rs1891158, rs7358335, rs12988652, rs3796547 rs7234917, rs6509345, rs966304, rs1515542, rs11631109, rs871688, rs4382847, rs9972541, rs13113975, rs4119478, rs1380862, rs7529518, rs785437, rs1140809, rs4830488, rs10458360, rs2738571, rs11634741, rs1950198, rs539357, rs16887736, rs7658048, rs11222496, rs2207790, rs12506850, rs4512641, rs2813532, rs6934898, rs582598, rs10191478, rs10486562, rs17395631, rs7525167, rs12637074, rs10887926, rs7485441, rs1944047, rs7178085, rs17318620, rs10489871, rs2691274, rs6962297, rs1827611, rs4806120, rs7164364, rs2293710, rs13017302, rs4570588, rs2386841, rs40485, rs524908, rs10795841, rs4273907, rs12612891, rs10496470, rs6755901, rs1943821, rs13319878, rs6957416, rs12552397, rs6489794, rs4346531, rs7777631, rs1046011, rs16988279, rs986472, rs10508422, rs9456490, rs1295683, rs2449600, rs7075945, rs9358913, rs1477886, rs753032, rs409558, rs4246742, rs10060513, rs17070292, rs10826398, rs17744022, rs7801918, rs885479, rs1863610, rs3805284, rs10832514, rs2509867, rs2070874, rs2339654, rs12903579, rs11610799, rs2272316, rs6961773, rs2078277, rs17324573, rs6760417, rs2911756, rs12233245, rs896615, rs4760442, rs2087724, rs439378, rs4833103, rs6539333, rs4423250, rs12594014, rs17123359, rs12505546, or rs585197.

The details of one of more embodiments of the disclosure are set forth in the description below. Other features or advantages of the present disclosure will be apparent from the detailed description of several embodiments and also from the appended claims.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein. In the figures:

FIG. 1 is a schematic of an exemplary chip with two fluidically isolated liquid containment regions. In this exemplary chip, binding partners for tPSA, fPSA, and hK2 are included in the first zone, and binding partners for iPSA, MIC-1, and MSMB are included in the second zone with replicate spots grouped together.

FIG. 2 is a schematic of an exemplary chip with two fluidically isolated liquid containment regions. In this exemplary chip, binding partners for tPSA, fPSA, and hK2 are included in the first zone, and binding partners for iPSA, MIC-1, and MSMB are included in the second zone with interspersed replicate spots.

FIG. 3 is a schematic of an exemplary solid state assay with multiple fluidically isolated liquid containment regions.

FIG. 4 is a chart with exemplary assay configurations requiring between two and six separate liquid containment regions.

FIG. 5 is a chart with exemplary assay configurations requiring between two and five separate liquid containment regions. Binding partners for iPSA are not present in any of the zones.

### DETAILED DESCRIPTION OF INVENTION

Aspects of the disclosure relate to improved solid-phase assay systems for determining levels of kallikrein markers. As a non-limiting example, kallikrein proteins (also called “kallikreins”) are a subgroup of enzymes capable of cleaving peptide bonds in proteins called serine proteases. Plasma kallikrein (KLKB1) as well as a group of fifteen closely related serine proteases known as tissue kallikrein-related peptidases (KLKs) are present in humans. KLK2, KLK3, KLK4, KLK5 and KLK14 are expressed in the prostate. Different forms of KLK3 (also known as Prostate-specific antigen; PSA; HK3, human kallikrein gene 3; and gamma-seminoprotein) and KLK2 (also known as hK2; human kallikrein 2; human glandular kallikrein; and hGK-1) are used as tumor markers proteins (markers) in several different forms for prostate cancer. The different (i.e., processed) forms of these proteins measured to determine prostate cancer risk may include: total prostate-specific antigen (tPSA), free prostate specific antigen (fPSA), intact prostate specific antigen (iPSA), total human Kallikrein 2 (thK2), and free human Kallikrein 2 (fhK2). PSA and hK2 have high homology including six regions where 15 consecutive amino acids are identical, and their structures are similar. Certain epitopes on the kallikrein proteins measured for prostate cancer risk are common epitopes and have therefore been highly problematic for the creation of multiplex assays for these markers. This application provides articles and methods for overcoming the difficulties associated with evaluating multiple different antigens and uses thereof for measuring the amounts of proteins having common or similar epitopes. Other aspects of the disclosure are related to methods for determining the probability of an event associated with prostate cancer based on evaluating amounts proteins having common or similar epitopes. Methods and assays disclosed herein may be employed by a healthcare provider for purposes of determining whether a subject being monitored has prostate cancer based on results of such assays. Methods and assays disclosed herein may also be employed by a healthcare provider for purposes of determining whether a subject being monitored may be at risk for developing prostate cancer based on results of such assays.

Assays described herein for determining levels of kallikrein markers generally involve the use of binding partners of the one or more binding partners that specifically bind to epitope(s) present on one or more kallikrein markers. As used herein, the term “binding partner”, with reference to a particular target molecule (e.g., a kallikrein protein), refers to an agent or molecule that specifically binds to the target molecule. Generally, the binding partner specifically binds to an epitope on the target molecule. In some embodiments, a binding partner is a protein, nucleic acid, glycoprotein, carbohydrate, or hormone. Specific examples of binding partners and targets include antibody/antigen, antibody fragment/antigen, antibody/hapten, antibody fragment/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

As a set of non-limiting examples, a binding partner may be any type of antibody including, but not limited to: an intact (i.e., full-length) polyclonal or monoclonal antibody, antigen-binding fragments thereof (such as Fab, Fab′, F(ab′)₂, or Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. A binding partner may be an antibody of any class, such as IgD, IgE, IgG, IgA, or IgM (or sub-class thereof, e.g., IgG1, IgG2, IgG3, IgG4, IgA1 and IgA2.

In certain embodiments, antibodies for use with the compositions and methods described herein specifically bind to an epitopes on their target molecule. The term “specifically binds,” as used herein, when referring to a binding partner (e.g., antibody or antibody fragment), refers to a binding reaction that is capable of discriminating between a target molecule and a non-target molecule. For example, a binding partner may specifically binds to a target molecule with at least a 2-fold greater affinity than non-target molecule, e.g., at least 4-fold, 5-fold, 6-fold, 7-fold, 8-fold, 9-fold, 10-fold, 20-fold, 25-fold, 50-fold, or 100-fold greater affinity. For example, in some embodiments, an antibody that specifically binds to hK2 will binds to hK2 with at least a 2-fold greater affinity than a non-hK2 protein or target (e.g., tPSA).

As used herein, “binding affinity” refers to the apparent association constant or K_{A}. The K_{A} is the reciprocal of the dissociation constant (K_{D}). In some embodiments, a binding partner described herein has a binding affinity (K_{D}) of at least 10⁻⁵, 10⁻⁶, 10⁻⁷, 10⁻⁸, 10⁻⁹, 10⁻¹⁰ M, or lower. An increased binding affinity corresponds to a decreased K_{D}. Higher affinity binding of a binding partner (e.g., an antibody) to a first molecule relative to a second molecule can be indicated by a higher K_{A} (or a smaller numerical value K_{D}) for binding the first target than the K_{A} (or numerical value K_{D}) for binding the second target. In such cases, the antibody has specificity for the first molecule (e.g., a protein in a first conformation or mimic thereof) relative to the second molecule (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

In some cases, an assay may take place in or on a chip. In some embodiments, a binding partner may be associated with (e.g., bound to) a surface of a solid support (e.g., the surface of a chip), and the complementary binding partner may be present in a fluid phase. Other solid-phase assays that involve affinity reaction between proteins or other biomolecules (e.g., DNA, RNA, carbohydrates), or non-naturally occurring molecules, can also be performed. Non-limiting examples of typical reactions that can be performed in or on a chip include chemical reactions, enzymatic reactions, immuno-based reactions (e.g., antigen-antibody), and cell-based reactions.

Sections of the solid support may be modified with one or multiple binding partners. The solid support may be linked in any manner to one or multiple binding partners. As a non-limiting example, the binding partners may be physisorbed or otherwise bound (e.g., directly) onto the surface of the substrate or covalently linked through appropriate coupling chemistry in any manner including, but not limited to: linkage through a epoxide on the surface, creation of an amido link (i.e., through NHS EDC chemistry) using a amine or carboxylic acid group present on the surface, linkage between a thiol and a thiol reactive group (i.e., a maleimide group), formation of a Schiff base between aldehyde and amines, reaction to an anhydride present on the surface, and/or through a photo-activatable linker.

In some embodiments, the instant disclosure provides one or more immunoassays that measure levels of prostate specific antigens, such as one or more of the following kallikrein markers: total prostate-specific antigen (tPSA), free prostate specific antigen (fPSA), intact prostate specific antigen (iPSA), total human Kallikrein 2 (thK2), and free human Kallikrein 2 (fhK2). In some embodiments, the instant disclosure provides one or more immunoassays that measure levels of prostate specific antigens, such as one or more of the following kallikrein markers: total prostate-specific antigen (tPSA), free prostate specific antigen (fPSA), total human Kallikrein 2 (thK2), and free human Kallikrein 2 (fhK2). In some embodiments, the immunoassays do not measure intact prostate specific antigen (iPSA).

In further embodiments, the one or more immunoassays also measure levels of macrophage inhibitory cytokine-1 (MIC-1) and beta-microseminoprotein (MSMB). In certain embodiments, the methods involve using a blood sample obtained from a subject to additionally conduct one or more immunoassays that measure levels of human prostatic acid phosphatase (PAP), early prostate cancer antigen (EPCA), glutathione S-transferase π (GSTP1), and/or a-methylacyl coenzyme A racemase (AMACR). In some embodiments, the methods involve using a blood sample obtained from a subject to additionally conduct one or more immunoassays that measure levels of additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG).

In some embodiments, the methods involve using a blood sample obtained from a subject to conduct one or more immunoassays that measure levels of prostate specific antigens, such as one or more of the following kallikrein proteins (markers): total prostate-specific antigen (tPSA), free prostate specific antigen (fPSA), intact prostate specific antigen (iPSA), total human Kallikrein 2 (thK2) free human Kallikrein 2 (fhK2). In some embodiments, the methods involve using a blood sample obtained from a subject to conduct one or more immunoassays that measure levels of prostate specific antigens, such as one or more of the following kallikrein proteins (markers): total prostate-specific antigen (tPSA), free prostate specific antigen (fPSA), total human Kallikrein 2 (thK2) free human Kallikrein 2 (fhK2). In some embodiments, the immunoassays do not measure intact prostate specific antigen (iPSA). In further embodiments, the methods involve using a blood sample obtained from a subject to additionally conduct one or more immunoassays that measure levels of macrophage inhibitory cytokine-1 (MIC-1) and beta-microseminoprotein (MSMB). In certain embodiments, the methods involve using a blood sample obtained from a subject to additionally conduct one or more immunoassays that measure levels of human prostatic acid phosphatase (PAP), early prostate cancer antigen (EPCA), glutathione S-transferase π (GSTP1), and/or a-methylacyl coenzyme A racemase (AMACR). In some embodiments, the methods involve using a blood sample obtained from a subject to additionally conduct one or more immunoassays that measure levels of additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG).

In some embodiments, a predictive model (e.g., a logistic regression model or a cubic spline model) is provided that incorporates plasma levels of tPSA, fPSA, iPSA, and/or hK2 (total and/or free hK2) to determine the probability of an event associated with prostate cancer in a subject. In some embodiments, a predictive model (e.g., a logistic regression model or a cubic spline model) is provided that incorporates plasma levels of tPSA, fPSA, and/or hK2 (total and/or free hK2) to determine the probability of an event associated with prostate cancer in a subject. In some embodiments, the predictive model does not incorporate levels of intact prostate specific antigen (iPSA). In further embodiments, a predictive model (e.g., a logistic regression model or a cubic spline model) is provided that additionally incorporates plasma levels of MIC-1 and/or MSMB to determine the probability of an event associated with prostate cancer in a subject. In certain embodiments, a predictive model (e.g., a logistic regression model or a cubic spline model) is provided that additionally incorporates plasma levels of human prostatic acid phosphatase (PAP), early prostate cancer antigen (EPCA), glutathione S-transferase π (GSTP1), and/or a-methylacyl coenzyme A racemase (AMACR). In some embodiments, a predictive model is provided that additionally incorporates plasma levels of additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HER3, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG). The predictive model may additionally incorporate information obtained, at least in part, during a clinical examination (e.g., a digital rectal exam or DRE) of the subject. The predictive model may further incorporate information from a category of single nucleotide polymorphisms (SNPs) related to prostate cancer, by measuring a presence or absence of each of a plurality of SNPs. The predictive model may also incorporate the age of the subject.

Accordingly, improved solid state assays for measuring levels of certain types of proteins (markers) and methods using the same are provided that may be useful for determining whether a subject should undergo an invasive prostate tissue biopsy.

Aspects of the disclosure provide methods of determining the probability that a prostate tissue biopsy obtained from a subject would contain detectable prostate cancer, e.g., aggressive prostate cancer. Such methods may involve subjecting a blood sample (e.g., serum, blood plasma, or whole blood) of a subject to an immunoassay that measures at least a level of total prostate specific antigen (tPSA) in the blood sample. If the tPSA level is above a threshold level, then the probability that a prostate tissue biopsy would contain detectable prostate cancer may be determined by weighting the measured level of tPSA and a parameter indicative of whether the subject has had a prior biopsy of prostate tissue. On the other hand, if the tPSA level is at or below the threshold level, then the probability that a prostate tissue biopsy would contain detectable prostate cancer may be determined by weighting measured levels of tPSA, fPSA, iPSA, hK2 (total and/or free hK2), MSMB, and/or MIC-1 and/or a parameter indicative of whether the subject has had a prior biopsy of prostate tissue. In certain cases, if the tPSA level is at or below the threshold level, then the probability that a prostate tissue biopsy would contain detectable prostate cancer may be determined by weighting measured levels of tPSA, fPSA, hK2 (total and/or free hK2), MSMB, and/or MIC-1 and/or a parameter indicative of whether the subject has had a prior biopsy of prostate tissue. In some embodiments, the determination of probability that a prostate tissue biopsy would contain detectable prostate cancer does not include weighting measured levels of intact prostate specific antigen (iPSA). In certain embodiments, the probability that a prostate tissue biopsy would contain detectable prostate cancer may be determined by additionally weighting measured levels of human prostatic acid phosphatase (PAP), early prostate cancer antigen (EPCA), glutathione S-transferase it (GSTP1), and/or a-methylacyl coenzyme A racemase (AMACR). In certain embodiments, the determination of probability that a prostate tissue biopsy would contain detectable prostate cancer may be determined by additionally weighting one or more additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG).

Accordingly, in some embodiments, methods provided herein may involve subjecting the blood sample to an immunoassay that measures levels of free prostate specific antigen (fPSA), intact prostate specific antigen (iPSA), human kallikrein 2 (free or total hK2), MSMB, and/or MIC-1 in the blood plasma sample. In some embodiments, methods provided herein may involve subjecting the blood sample to an immunoassay that measures levels of free prostate specific antigen (fPSA), human kallikrein 2 (free or total hK2), MSMB, and/or MIC-1 in the blood plasma sample. In certain embodiments, methods provided herein may additionally involve subjecting the blood plasma sample to an immunoassay that measures levels of human prostatic acid phosphatase (PAP), early prostate cancer antigen (EPCA), glutathione S-transferase π (GSTP1), and/or a-methylacyl coenzyme A racemase (AMACR). In some embodiments, the methods may additionally involve subjecting the sample to one or more additional immunoassays that measure levels of additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG). In some embodiments, the methods provided herein do not include measuring iPSA.

In some embodiments, the immunoassay does not measure levels of intact prostate specific antigen (iPSA). In some embodiments, the probability is further determined by weighting a parameter indicative of the subject's age. In some embodiments, the probability is further determined by weighting at least one clinical factor, such as, for example, one or more parameters indicative of the outcome of a digital rectal examination performed on the subject. In some embodiments, the at least one clinical factor is selected from: number of prostate tissue biopsies performed on the subject to date; results of prior prostate tissue biopsies performed on the subject to date; occurrence of any negative biopsy since an initial diagnosis of non-aggressive prostate cancer; occurrence of any negative biopsy in one-year prior to obtaining the blood sample; total number of biopsies since an initial diagnosis of non-aggressive prostate cancer; prostate volume on prior biopsy; number of positive cores on prior biopsy; percent positive cores on prior biopsy; cross-sectional area of cancer in biopsy core sections; maximum cross-sectional area of cancer in any biopsy core sections; PSA density; race of subject; family history of prostate cancer; maximum percent of positive cores from any prior biopsy; and maximum number of positive cores from any prior biopsy.

In some embodiments, the threshold level of tPSA used for model selection is a level that indicates whether using tPSA alone, or together with certain subject specific information (e.g., prior biopsy status), would be sufficient for purposes of establishing a probability that a prostate tissue biopsy would contain detectable prostate cancer. In some embodiments, the threshold level is 5 ng/mL, 10 ng/mL, 15 ng/mL, 20 ng/mL, 25 ng/mL, 30 ng/mL, 35 ng/mL or 40 ng/mL. Because tPSA levels combined with certain subject specification information, particularly prior biopsy status, may be enough to make informative predictions, in some embodiments, it may be cost effective not to perform immunoassays to detect other antigens before first determining levels of tPSA. However, in some embodiments, levels of tPSA may be determined in parallel or together with other protein (marker) levels, e.g., fPSA, iPSA, hK2 (free or total), MSMB, and/or MIC-1. In some embodiments, levels of tPSA may be determined in parallel or together with other protein (marker) levels, e.g., fPSA, hK2 (free or total), MSMB, and/or MIC-1. In some embodiments, the levels of tPSA are not determined in parallel or together with the level of iPSA. In certain embodiments, additional proteins (markers) may include human prostatic acid phosphatase (PAP), early prostate cancer antigen (EPCA), glutathione S-transferase π (GSTP1), and/or a-methylacyl coenzyme A racemase (AMACR). In some embodiments, additional proteins (markers) may be selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG).

In some embodiments, multiple kallikrein protein (marker) levels (e.g., levels of two or more of tPSA, fPSA, iPSA, and hK2 (total or free)) are determined in parallel in the same assay. In some embodiments, multiple kallikrein protein (marker) levels (e.g., levels of two or more of tPSA, fPSA, and hK2 (total or free)) are determined in parallel in the same assay. In some embodiments, the assay does not include iPSA. In other embodiments, such antigen levels are determined in separate assays. In some embodiments, antigen levels are determined from the same original blood draw (e.g., a venous blood draw) from a subject. In some embodiments, antigen levels are determined from different blood draws. In some embodiments, antigen levels are determined using plasma preparations from the same or different blood draws. In some embodiments, one or more antigen levels are determined using a plasma preparation and one or more other antigens are determined using a different type of blood preparation, e.g., serum. Blood plasma is a pale-yellow liquid component of blood. In some embodiments, blood plasma may be prepared by spinning a tube of blood containing an anticoagulant (e.g., Heparin, EDTA, etc.) in a centrifuge until blood cells and debris move to the bottom of the tube, after which the blood plasma may be poured or drawn off.

Methods are provided herein for determining whether a subject is a candidate for a prostate tissue biopsy. Such methods may involve a physician or health care provider obtaining a blood sample from a subject and determining the probability that the prostate tissue biopsy would contain detectable prostate cancer (e.g., aggressive prostate cancer) based, at least in part, on measured levels of antigens determined using the blood sample. The blood sample may be processed locally (e.g., within the same health care facility or business that the subject is being evaluated) or may send it out to an external or third-party laboratory or facility for processing and analysis. If a tPSA level measured using the blood sample is above a threshold level, the probability may be determined based on weighting the tPSA level. Otherwise, if the tPSA level is at or below the threshold level, the probability may be based on weighting levels of tPSA, fPSA, iPSA, and hK2 (total or free) measured using the blood sample. In some embodiments, if the tPSA level is at or below the threshold level, the probability may be based on weighting levels of tPSA, fPSA, and hK2 (total or free) measured using the blood sample. In some embodiments, the probability is not based on weighting the level of iPSA. In further embodiments, the probability may be additionally based on weighting levels of human prostatic acid phosphatase (PAP), early prostate cancer antigen (EPCA), glutathione S-transferase π (GSTP1), and/or a-methylacyl coenzyme A racemase (AMACR). In some embodiments, the probability may be additionally based on weighting levels of additional proteins (markers) selected from the group consisting of: 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and zinc-alpha2-glycoprotein (ZAG). In either case, the probability is typically also based on weighting at least one clinical factors, such as, for example, a parameter indicative of whether the subject had a prior biopsy of prostate tissue. The physician or healthcare provider may determine whether the subject is a candidate for the prostate tissue biopsy based on the probability that the prostate tissue biopsy will contain detectable prostate cancer.

In some embodiments, if a subject is determined to be a candidate for a prostate tissue biopsy, then the physician or health care provider may obtain or order to be obtained a prostate tissue biopsy from the subject and determine whether the subject has prostate cancer based on an analysis of the prostate tissue biopsy. The prostate tissue biopsy may be analyzed using any appropriate method including, for example, a cytological or histological analysis. The tissue sample may be characterized based on its clinical stage of cancer. The sample may be characterized based on a Gleason grade. Gleason 3+3 (6.0) corresponds to a tumor of low grade and a favorable prognosis. Gleason 3+4 (7.0) and 3+5 (8.0) typically correspond to tumors that have tissue of primarily low grade transformation with some high grade transformation. Gleason 4+3 (7.0) and 5+3 (8.0) typically correspond to tumor that have tissue of primarily high grade transformation with some low grade transformation. Gleason 4+4 (8.0), 4+5 (9.0), (9.0), and 5+5 (10.0) corresponds to high grade tumors. Accordingly, in some embodiments, the prostate cancer comprises high grade cancer (e.g., Gleason≥7.0).

In some embodiments, the probability that a subject has prostate cancer is based on a linear combination of the data based on the measured protein (marker) level(s). In some embodiments, the probability that a subject has prostate cancer is further determined by weighting a cubic spline term based on the measured protein (marker) level(s).

Further aspects of the disclosure relate to a method for determining a probability of an event associated with prostate cancer, the event being an upgrade from a non-aggressive prostate cancer to an aggressive prostate cancer. In some embodiments, the methods involve receiving, via an input interface, information indicative of levels of one or more kallikrein proteins (markers) selected from: tPSA, fPSA, iPSA, and hK2 (free hK2 or total hK2) in a blood plasma sample of a subject previously diagnosed as having a non-aggressive prostate cancer; receiving, via an input interface, information about at least one clinical factor of the subject evaluating, using at least one processor, a logistic regression model based, at least in part, on the received information to determine a probability of an event associated with prostate cancer in the subject, wherein evaluating the logistic regression model comprises: determining the probability of the event associated with prostate cancer based, at least in part, on the information indicative of levels of one or more of tPSA, fPSA, iPSA, hK2 (free or total hK2), MSMB, and/or MIC-1 and the information about the at least one clinical factor; and outputting an indication of the probability of the event associated with prostate cancer. In some embodiments, the methods involve receiving, via an input interface, information indicative of levels of one or more kallikrein proteins (markers) selected from: tPSA, fPSA, and hK2 (free hK2 or total hK2) in a blood plasma sample of a subject previously diagnosed as having a non-aggressive prostate cancer; receiving, via an input interface, information about at least one clinical factor of the subject evaluating, using at least one processor, a logistic regression model based, at least in part, on the received information to determine a probability of an event associated with prostate cancer in the subject, wherein evaluating the logistic regression model comprises: determining the probability of the event associated with prostate cancer based, at least in part, on the information indicative of levels of one or more of tPSA, fPSA, hK2 (free or total hK2), MSMB, and/or MIC-1 and the information about the at least one clinical factor; and outputting an indication of the probability of the event associated with prostate cancer. In some embodiments, the methods do not make use of information or data about or the level of iPSA.

Further aspects of the disclosure relate to a computer for determining a probability of an event associated with prostate cancer, the event being an upgrade from a non-aggressive prostate cancer to an aggressive prostate cancer. In some embodiments, the computer comprises an input interface configured to receive information indicative of levels of one or more kallikrein proteins (markers) selected from: tPSA, fPSA, iPSA, and hK2 (free or total hK2) in a blood sample from a subject and information about at least one clinical factor of the subject; at least one processor programmed to evaluate a logistic regression model based, at least in part, on the received information to determine a probability of an event associated with prostate cancer in the subject; and an output interface configured to output an indication of the probability of the event associated with prostate cancer, wherein the event associated with prostate cancer is an upgrade from a non-aggressive prostate cancer to an aggressive prostate cancer. In some embodiments, the computer comprises an input interface configured to receive information indicative of levels of one or more kallikrein proteins (markers) selected from: tPSA, fPSA, and hK2 (free or total hK2) in a blood sample from a subject and information about at least one clinical factor of the subject; at least one processor programmed to evaluate a logistic regression model based, at least in part, on the received information to determine a probability of an event associated with prostate cancer in the subject; and an output interface configured to output an indication of the probability of the event associated with prostate cancer, wherein the event associated with prostate cancer is an upgrade from a non-aggressive prostate cancer to an aggressive prostate cancer.

In some embodiments, the computer does not make use of information or data about or the level of iPSA. In some embodiments, evaluating the logistic regression model comprises: determining the probability of the event associated with prostate cancer based, at least in part, on the information indicative of levels of one or more of tPSA, fPSA, iPSA, hK2 (free or total hK2), MSMB, and/or MIC-1 and the information about the at least one clinical factor. In some embodiments, evaluating the logistic regression model comprises: determining the probability of the event associated with prostate cancer based, at least in part, on the information indicative of levels of one or more of tPSA, fPSA, hK2 (free or total hK2), MSMB, and/or MIC-1 and the information about the at least one clinical factor. In some embodiments, evaluating the logistic regression model does not comprise use of information indicative of the level of iPSA.

Further aspects of the disclosure relate to a system for determining a probability of an event associated with prostate cancer, the event associated with prostate cancer being an upgrade from a non-aggressive prostate cancer to an aggressive prostate cancer. In some embodiments, the system comprises: a) a detector configured to measure levels of one or more kallikrein proteins (markers) selected from: tPSA, fPSA, iPSA, and hK2 (free or total hK2) in a blood plasma sample of a subject; and b) a computer in communication (e.g., electronic communication or wireless communication) with the detector. In some embodiments, the computer includes an input interface configured to receive information from the detector indicative of the measured levels of one or more of tPSA, fPSA, iPSA, hK2 (free or total hK2), MSMB, and/or MIC-1, and to receive information about at least one clinical factor of the subject; at least one processor programmed to evaluate a logistic regression model based, at least in part, on the received information to determine a probability of an event associated with prostate cancer in the subject; and an output interface configured to output an indication of the probability of the event associated with prostate cancer. In some embodiments, aspects of the disclosure relate to a system for determining a probability of an event associated with prostate cancer, the event associated with prostate cancer being an upgrade from a non-aggressive prostate cancer to an aggressive prostate cancer. In some embodiments, the system comprises: a) a detector configured to measure levels of one or more kallikrein proteins (markers) selected from: tPSA, fPSA, and hK2 (free or total hK2) in a blood plasma sample of a subject; and b) a computer in communication (e.g., electronic communication or wireless communication) with the detector.

In some embodiments, the computer includes an input interface configured to receive information from the detector indicative of the measured levels of one or more of tPSA, fPSA, hK2 (free or total hK2), MSMB, and/or MIC-1, and to receive information about at least one clinical factor of the subject; at least one processor programmed to evaluate a logistic regression model based, at least in part, on the received information to determine a probability of an event associated with prostate cancer in the subject; and an output interface configured to output an indication of the probability of the event associated with prostate cancer. In some embodiments, the system does not make use of information or data about or the level of iPSA.

In some embodiments, evaluating the logistic regression model involves: determining the probability of the event associated with prostate cancer based, at least in part, on the information indicative of levels of one or more of tPSA, fPSA, iPSA, hK2 (free or total hK2), MSMB, and/or MIC-1 and the information about the at least one clinical factor. In some embodiments, evaluating the logistic regression model involves: determining the probability of the event associated with prostate cancer based, at least in part, on the information indicative of levels of one or more of tPSA, fPSA, hK2 (free or total hK2), MSMB, and/or MIC-1 and the information about the at least one clinical factor. In some embodiments, evaluating the logistic regression model does not comprise use of information indicative of the level of iPSA.

Still further aspects of the disclosure relate to a computer-readable storage medium encoded with a plurality of instructions that, when executed by a computer, perform a method for determining a probability of an event associated with prostate cancer, the event being an upgrade from a non-aggressive prostate cancer to an aggressive prostate cancer. In some embodiments, the method involves evaluating a logistic regression model based, at least in part, on information indicative of levels of one or more kallikrein proteins (markers) selected from: tPSA, fPSA, iPSA, and hK2 (free or total hK2) in a blood plasma sample of a subject and information about at least one clinical factor of the subject to determine a probability of an event associated with prostate cancer in the subject, and outputting an indication of the probability of the event associated with prostate cancer. In some embodiments, evaluating the logistic regression model involves: determining the probability of the event associated with prostate cancer based, at least in part, on the information indicative of levels of one or more of tPSA, fPSA, iPSA, hK2 (free or total hK2), MSMB, and/or MIC-1 and the information about the at least one clinical factor. In some embodiments, the method involves evaluating a logistic regression model based, at least in part, on information indicative of levels of one or more kallikrein proteins (markers) selected from: tPSA, fPSA, and hK2 (free or total hK2) in a blood plasma sample of a subject and information about at least one clinical factor of the subject to determine a probability of an event associated with prostate cancer in the subject, and outputting an indication of the probability of the event associated with prostate cancer. In some embodiments, evaluating the logistic regression model involves: determining the probability of the event associated with prostate cancer based, at least in part, on the information indicative of levels of one or more of tPSA, fPSA, hK2 (free or total hK2), MSMB, and/or MIC-1 and the information about the at least one clinical factor.

### Immunoassays

Levels of specific antigens [e.g., kallikrein proteins (markers) such as tPSA, iPSA, fPSA, and/or hK2 (total hK2 or free hK2) as well as additional proteins (markers) such as MSMB1, MIC-1, PAP, EPCA, GSTP1, AMACR, 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and/or zinc-alpha2-glycoprotein (ZAG)] can be assessed by any appropriate method. In certain embodiments, levels of iPSA are not assessed. In some embodiments, antibodies or antigen-binding fragments are provided that are suited for use in immunoassays. Immunoassays utilizing such antibody or antigen-binding fragments may competitive and non-competitive immunoassays in either a direct or indirect formats. Non-limiting examples of such immunoassays are Enzyme Linked Immunoassays (ELISA), radioimmunoassays (RIA), sandwich assays (immunometric assays), flow cytometry-based assays, western blot assays, immunoprecipitation assays, immunohistochemistry assays, immuno-microscopy assays, lateral flow immuno-chromatographic assays, and proteomics arrays.

Probes including antigens or antibodies or antigen-binding fragments that bind to the specific antigens of interest can be immobilized, e.g., by binding to solid supports (e.g., a chip, carrier, membrane, columns, proteomics array, etc.). In one set of embodiments, a material used to form the solid support has an optical transmission of greater than 90% between 400 and 800 nm wavelengths of light (e.g., light in the visible range). Optical transmission may be measured through a material having a thickness of, for example, about 2 mm (or in other embodiments, about 1 mm or about 0.1 mm). In some instances, the optical transmission is greater than or equal to 80%, greater than or equal to 85%, greater than or equal to 88%, greater than or equal to 92%, greater than or equal to 94%, or greater than or equal to 96% between 400 and 800 nm wavelengths of light. In some embodiments, the material used to form the solid support has an optical transmission of less than or equal to 99.9%, less than or equal to 96%, less than or equal to 94%, less than or equal to 92%, less than or equal to 90%, less than or equal to 85%, less than or equal to 80%, less than or equal to 50%, less than or equal to 30%, or less than or equal to 10% between 400 and 800 nm wavelengths of light. Combinations of the above-referenced ranges are also possible. Non-limiting examples of solid support materials include glass, plastics, elastomeric materials, membranes, or other suitable materials for performing immunoassays. The solid support may be formed from one material, or it may be formed from two or more materials.

For example, the probes can be antibodies (or antibody fragments) with specificity towards protein of interest including PSA, hK2 (total or free hK2), MSMB or MIC1 (also known as GDF-15). Antibodies against other proteins known to be associated with medical conditions of interest may be used. Medical conditions of interest include, but are not limited to: benign prostatic hyperplasia, prostate cancer, aggressive prostate cancer, high-grade prostate cancer, metastatic prostate cancer, and lethal prostate cancer. PSA and hK2 can be found in human clinical samples in different isoforms. Commonly PSA refers to total PSA, the sum of PSA in all its detectable forms by immunoassay: free or complexed to α-antichymotrypsin or alpha-2-macroglobulin, intact (single chain) or nicked, with or without pre-pro amino sequence (or a partial prepro amino acid sequence). Similarly hK2 can be found as free or complexed. Antibodies can be selected to bind to epitopes that are specific for the different forms of PSA or hK2. In a sandwich assay format, the selectivity for a given form of PSA or hK2 is achieved by combining the specificity of two antibodies. For instance intact PSA can be detected by exposing the sample to a free PSA capture antibody, and after a wash, reacting with an antibody specific to intact PSA. The antibodies can be selected from a list of known clones, or can be new antibodies with known affinities to the protein of interest.

Specific solid support materials may include, but are not limited to: any type of glass (e.g., fused silica, borosilicate glass, Pyrex®, or Duran®). In one embodiment, the solid support is a glass chip. The solid support may also comprise a non-glass substrate coated with a glass film dioxide produced by a process such as sputtering, oxidation of silicon, or through reaction of silane reagents. The glass surface may be further modified with functionalized silane reagents including, for example: amine-terminated silanes (aminopropyltriethoxy silane) and epoxide-terminated silanes (glycidoxypropyltrimethoxysilane).

Additional specific solid support materials may include, but are not limited to: thermoplastic polymers and may comprise one or more of: polystyrene, polycarbonate, polymethylmetacrylate, cyclic olefin copolymers, polyethylene, polypropylene, polyvinyl chloride, polyvinylidene difluoride, any fluoropolymers (e.g., polytetrafluoroethylene, also known as Teflon®), polylactic acid, poly(methyl methacrylate) (also known as PMMA or acrylic; e.g., Lucite®, Perspex®, and Plexiglas®), and acrylonitrile butadiene styrene.

Additional specific solid support materials may include, but are not limited to: one or more elastomeric materials including polysiloxanes (silicones such as polydimethylsiloxane) and rubbers (polyisoprene, polybutadiene, chloroprene, styrene-butadiene, nitrile rubber, polyether block amides, ethylene-vinyl acetate, epichlorohydrin rubber, isobutene-isoprene, nitrile, neoprene, ethylene-propylene, and hypalon).

Additional specific solid support materials may include, but are not limited to: one or more membrane substrates such as dextran, amyloses, nylon, Polyvinylidene fluoride (PVDF), fiberglass, and natural or modified celluloses (e.g., cellulose, nitrocellulose, CNBr-activated cellulose, and cellulose modified with polyacrylamides, agaroses, and/or magnetite). The nature of the support can be either fixed or suspended in a solution (e.g., beads).

Sections of the solid support may be modified with one or multiple capture probes. The solid support may be linked in any manner to one or multiple capture probes. As a non-limiting example, the capture probe may be physisorbed or otherwise bound (e.g., directly) onto the surface of the substrate or covalently linked through appropriate coupling chemistry in any manner including, but not limited to: linkage through a epoxide on the surface, creation of an amido link (i.e., through NHS EDC chemistry) using a amine or carboxylic acid group present on the surface, linkage between a thiol and a thiol reactive group (i.e., a maleimide group), formation of a Schiff base between aldehyde and amines, reaction to an anhydride present on the surface, and/or through a photo-activatable linker.

The solid support may also contain one or more additional chemical groups suitable to achieve covalent coupling to a one or more probes. These additional chemical groups may any such group or molecule known in the art. As a non-limiting example, the additional group or molecule may be streptavidin (or derivatives such as neutravidin, avidin, and captavidin) are bound to the surface as indicated above, and then reacted with a biotinylated probe.

The probe may be a protein with naturally occurring amino acids or artificial amino acids, one or more nucleic acids made of naturally occurring based or artificial bases (including, for example, DNA or RNA), sugars, carbohydrates, one or more small molecules (including, but not limited to one or more of: a vitamin, hormone, cofactor, heme group, chelate, fatty acid, or other known small molecule, and/or a phage.

The probes may be applied to the surface of the substrate by deposition of a droplet at a pre-defined location in any manner and using any device including, but not limiting to: the use of a pipette, a liquid dispenser, plotter, nano-spotter, nano-plotter, arrayer, spraying mechanism or other suitable fluid handling device.

In some embodiments, the material and dimensions (e.g., thickness) of a chip and/or cover are chosen such that it is substantially impermeable to water vapor. For instance, a chip may include a cover comprising a material known to provide a high vapor barrier, such as metal foil, certain polymers, certain ceramics and combinations thereof. Examples of materials having low water vapor permeability are provided below. In other cases, the material is chosen based at least in part on the shape and/or configuration of the chip. For instance, certain materials can be used to form planar devices whereas other materials are more suitable for forming devices that are curved or irregularly shaped.

A material used to form all or portions of a section or component of a device may have, for example, a water vapor permeability of less than about 5.0 g·mm/m²˜d, less than about 4.0 g·mm/m²˜d, less than about 3.0 g·mm/m²˜d, less than about 2.0 g·mm/m²˜d, less than about 1.0 g·mm/m²˜d, less than about 0.5 g·mm/m²˜d, less than about 0.3 g·mm/m²˜d, less than about 0.1 g·mm/m²˜d, or less than about 0.05 g·mm/m²˜d. In some cases, the water vapor permeability may be, for example, between about 0.01 g·mm/m²˜d and about 2.0 g·mm/m²˜d, between about 0.01 g·mm/m²˜d and about 1.0 g·mm/m²˜d, between about 0.01 g·mm/m²˜d and about 0.4 g·mm/m²˜d, between about 0.01 g·mm/m²˜d and about 0.04 g·mm/m²˜d, or between about 0.01 g·mm/m²˜d and about 0.1 g·mm/m²˜d. The water vapor permeability may be measured at, for example, 40° C. at 90% relative humidity (RH). Combinations of materials with any of the aforementioned water vapor permeabilities may be used in the instant assays or methods.

In some embodiments, the material and dimensions of a chip and/or cover vary. For example, the chip may be configured to provide one or more regions (e.g., liquid containment regions). In certain embodiments, the chip may be configured to provide two or more regions (e.g., liquid containment regions). In certain embodiments, two or more of the regions are fluidically separated from other regions. In one embodiment, all of the regions are fluidically separated from other regions. The chip may comprise any number of liquid containment regions. As a non-limiting example, the chip may comprise one, two, three, four, five, six, seven, eight, nine, or ten liquid containment regions, each of which may be fluidically separated from one another. In other embodiments, the chip may comprise one, two, three, four, five, six, seven, eight, nine, or ten liquid containment regions that are fluidically connected to one another.

In some instances, a chip may be comprised of a combination of two or more materials, such as the ones listed above. For instance, portions of the chip may be formed in polystyrene or other polymers (e.g., by injection molding). In one embodiment, biocompatible tape may be used to separate and/or seal one or more liquid containment regions of the chip. The biocompatible tape or flexible material may include a material known to improve vapor barrier properties (e.g., metal foil, polymers or other materials known to have high vapor barriers), and may optionally allow access to inlets and outlets by puncturing or unpeeling the tape. A variety of methods can be used to join multiple layers of the chip, including but not limited to, the use of adhesives, use adhesive tapes, gluing, bonding, lamination of materials, or by mechanical methods (e.g., clamping, snapping mechanisms, etc.).

A chip described herein may have any suitable volume for carrying out an analysis such as a chemical and/or biological reaction or other process. The entire volume of a chip includes, for example, any reagent storage areas, analysis regions, liquid containment regions, waste areas, as well as one or more identifiers (discussed in further detail below). In some embodiments, small amounts of reagents and samples are used and the entire volume of the a liquid containment region is, for example, less than or equal to 10 mL, less than or equal to 5 mL, less than or equal to 1 mL, less than or equal to 500 μL, less than or equal to 250 μL, less than or equal to 100 μL, less than or equal to 50 μL, less than or equal to 25 μL, less than or equal to 10 μL, less than or equal to 5 μL, or less than or equal to 1 μL. In some embodiments, small amounts of reagents and samples are used and the entire volume of the a liquid containment region is, for example, at least 10 mL, at least 5 mL, at least 1 mL, at least 500 μL, at least 250 μL, at least 100 μL, at least 50 μL, at least 25 μL, at least 10 μL, at least 5 μL, or at least 1 μL. Combinations of the above-referenced values are also possible.

The length and/or width of the chip may be, for example, less than or equal to 300 mm, less than or equal to 200 mm, less than or equal to 150 mm, less than or equal to 100 mm, less than or equal to 95 mm, less than or equal to 90 mm, less than or equal to 85 mm, less than or equal to 80 mm, less than or equal to 75 mm, less than or equal to 70 mm, less than or equal to 65 mm, less than or equal to 60 mm, less than or equal to 55 mm, less than or equal to 50 mm, less than or equal to 45 mm, less than or equal to 40 mm, less than or equal to 35 mm, less than or equal to 30 mm, less than or equal to 25 mm, or less than or equal to 20 mm. In some embodiments, the length and/or width of the chip may be, for example, at least 300 mm, at least 200 mm, at least 150 mm, at least 100 mm, at least 95 mm, at least 90 mm, at least 85 mm, at least 80 mm, at least 75 mm, at least 70 mm, at least 65 mm, at least 60 mm, at least 55 mm, at least 50 mm, at least 45 mm, at least 40 mm, at least 35 mm, at least 30 mm, at least 25 mm, or at least 20 mm. Combinations of the above-referenced values are also possible. In some embodiments, the thickness of the chip may be, for example, less than or equal to 5 mm, less than or equal to 3 mm, less than or equal to 2 mm, less than or equal to 1 mm, less than or equal to 0.9 mm, less than or equal to 0.8 mm, less than or equal to 0.7 mm, less than or equal to 0.5 mm, less than or equal to 0.4 mm, less than or equal to 0.3 mm, less than or equal to 0.2 mm, or less than or equal to 0.1 mm. In some embodiments, the thickness of the chip may be, for example, at least 5 mm, at least 3 mm, at least 2 mm, at least 1 mm, at least 0.9 mm, at least 0.8 mm, at least 0.7 mm, at least 0.5 mm, at least 0.4 mm, at least 0.3 mm, at least 0.2 mm, or at least 0.1 mm. Combinations of the above-referenced values are also possible. One or more chips may be analyzed at the same time by any suitable device. An adapter may be used with the one or more chips in order to insert and securely hold them in the analyzer.

It should be understood that the chips and their respective components described herein are exemplary and that other configurations and/or types of chips and components can be used with the systems and methods described herein.

The binding of a substrate (e.g., to detect the binding of a protein of interest including, but not limited to, antigen-bound antibody complexes) may be quantified by interrogating an active molecule bound to the tracer antibody. In a multiplexed format, where more than one assay is being performed on a continuous area, the signals associated with each assay must be differentiable from the other assays. Any suitable strategy known in the art may be used including, but not limited to: (1) using a label with substantially non-overlapping spectral and/or electrochemical properties: (2) using a signal amplification chemistry that remains attached or deposited in close proximity to the tracer itself.

In some embodiments, labeled antibodies or antigen binding fragments may be used as tracers to detect antigen bound antibody complexes. Examples of the types of labels which can be used to generate tracers include enzymes, radioisotopes, colloidal metals, fluorescent compounds, magnetic, chemiluminescent compounds, electrochemiluminescent groups, metal nanoparticles, and bioluminescent compounds. Radiolabeled antibodies are prepared in known ways by coupling a radioactive isotope such as ¹⁵³Eu, ³H, ³²P, ³⁵S, ⁵⁹Fe, or ¹²⁵I, which can then be detected by gamma counter, scintillation counter or by autoradiography. As discussed herein, antibodies and antigen-binding fragments may alternatively be labeled with enzymes such as yeast alcohol dehydrogenase, horseradish peroxidase, alkaline phosphatase, and the like, then developed and detected spectrophotometrically or visually. The label may be used to react a chromogen into a detectable chromophore (especially if the chromogen is a precipitating dye).

Suitable fluorescent labels may include, but are not limited to: fluorescein, fluorescein isothiocyanate, fluorescamine, rhodamine, Alexa Fluor® dyes (such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, or Alexa Fluor® 790), cyanine dyes including, but not limited to: Cy2, Cy3, Cy3.5, Cy5, Cy5.5, Cy7, and Cy7.5, and the like. The labels may also be time-resolved fluorescent (TRF) atoms (e.g., Eu or Sr with appropriate ligands to enhance TRF yield). More than one fluorophore capable of producing a fluorescence resonance energy transfer (FRET) may also be used. Suitable chemiluminescent labels may include, but are not limited to: acridinium esters, luminol, imidazole, oxalate ester, luciferin, and any other similar labels.

Suitable electrochemiluminescent groups for use may include, as a non-limiting example: Ruthenium and similar groups. A metal nanoparticle may also be used as a label. The metal nanoparticle may be used to catalyze a metal enhancement reaction (such as gold colloid for silver enhancement). Any of the labels described herein or known in the field may be linked to the tracer using covalent or non-covalent means. The label may be presented on or inside an object like a bead (including, for example, a plain bead, hollow bead, or bead with a ferromagnetic core), and the bead is then attached to the tracer antibody. The label may also be a nanoparticle including, but not limited to, an up-converting phosphorescent system, nanodot, quantum dot, nanorod, and/or nanowire. The label linked to the antibody may also be a nucleic acid, which might then be amplified (e.g., using PCR) before quantification by one or more of optical, electrical or electrochemical means.

An antigen is a molecule or a portion of a molecule that can have antibodies generated against it. Antigens are usually peptides, polysaccharides or lipids and may originate from within the body (a “self-antigen”) or from the external environment (a “non-self-antigen”).

An epitope (also known as an antigenic determinant) is the part of the antigen recognized (or bound by) an antibody. For example, the epitope is the specific piece of the antigen to which an antibody binds. The part of an antibody that binds to the epitope is called a paratope. An epitope may be a conformational epitope (composed of discontinuous amino acids or sections of the antigen) or a linear epitope (composed of continuous amino acids). Certain proteins share segments of high sequence homology and/or structural similarity. These similar proteins may have common epitopes (i.e., the epitopes on different antibodies may be bound by the same antibody). Further, a protein that has been processed differentially (i.e., a protein that has gone a further enzymatic process) may share some, but not all epitopes with its pre-processing form. Examples of different epitopes that may be added or removed during processing may include an N-terminal signal peptide (as seen, for example, on pre-pro-peptides) or the changes seen when an inactive protein (e.g., a pro-peptide) is turned into an active form by post-translational modification. As a specific example, human blood can contain a variety of forms of PSA such as different forms of free PSA (nicked, intact and proPSA) and complexed PSA (PSA complexed with protein C inhibitor (PCI, encoded by SERPINA2), a protease inhibitor). Since the different forms of PSA have common (or overlapping) epitopes, analysis of the levels of these forms may present difficulty.

Kallikreins are a subgroup of enzymes capable of cleaving peptide bonds in proteins called serine proteases. Humans have plasma kallikrein (KLKB1; located at chromosome 4q34-35) as well as a group of fifteen closely related serine proteases known as tissue kallikrein-related peptidases (KLKs). The KLKs are located at chromosome 19q13 and form the largest contiguous cluster of proteases within the human genome. KLK2, KLK3, KLK4, KLK5 and KLK14 are expressed in the prostate. KLK3 (also known as Prostate-specific antigen; PSA; HK3, human kallikrein gene 3; and gamma-seminoprotein) and KLK2 (also known as hK2; human kallikrein 2; human glandular kallikrein; and hGK-1) are used as tumor proteins (markers) for prostate cancer. PSA and hK2 have high homology including six regions where 15 consecutive amino acids are identical, and their structures are similar. See: Hentty et al. Ann Med 1994; 26:165-71 and Henttu and Vihko Biochem. Biophys. Res. Commun. 1989; 160, 903-910, each of which is herein incorporated by reference in its entirety. Therefore, certain epitopes on PSA and hK2 are common epitopes.

An immunoassay may comprise contacting a sample, e.g., a plasma sample, containing an antigen with an antibody, or antigen-binding fragment (e.g., F(ab), F(ab)₂), under conditions enabling the formation of binding complexes between antibody or antigen-binding fragment and antigen. In some embodiments, a plasma sample is contacted with an antibody or antigen-binding fragment under conditions suitable for binding of the antibody or antigen-binding fragment to a target antigen, if the antigen is present in the sample. This may be performed in a suitable reaction chamber, such as a tube, plate well, membrane bath, cell culture dish, microscope slide, and other chamber. In some embodiments, an antibody or antigen-binding fragment is immobilized on a solid support. An antibody or antigen binding fragments that binds to an antigen in a sample may be referred to as a capture antibody. In some embodiments, the capture antibody comprises a tag (e.g., a biotin label) that facilitates its immobilization to a solid support by an interaction involving the tag (e.g., a biotin-streptavidin interaction in which the streptavidin is immobilized to a solid support). In some embodiments, the solid support is the surface of a reaction chamber. In some embodiments, the solid support is of a polymeric membrane (e.g., nitrocellulose strip, Polyvinylidene Difluoride (PVDF) membrane, etc.). In other embodiments, the solid support is a biological structure (e.g., bacterial cell surface). Other exemplary solid supports are disclosed herein and will be apparent to one of ordinary skill in the art.

In some embodiments, the antibody or antigen-binding fragment is immobilized on the solid support prior to contacting with the antigen. In other embodiments, immobilization of the antibody and antigen-binding fragment is performed after formation of binding complexes. In still other embodiments, the antigen is immobilized on a solid support prior to formation of binding complexes. In some embodiments, a tracer may be added to the reaction chamber to detect immobilized binding complexes. In some embodiments, the tracer comprises a detectably labeled secondary antibody directed against the antigen. In some embodiments, the tracer comprises a detectably labeled secondary antibody directed against the capture antibody. In some embodiments, the primary antibody or antigen-binding fragment is itself detectable labeled.

In one embodiment, immunoassay methods disclosed herein comprise immobilizing antibodies or antigen-binding fragments to a solid support (e.g., a chip); applying a sample (e.g., a plasma sample) to the solid support under conditions that permit binding of antigen to the antibodies or antigen-binding fragment, if present in the sample; removing the excess sample from the solid support; applying a tracer (e.g., detectably labeled antibodies or antigen-binding fragments) under conditions that permit binding of the tracer to the antigen-bound immobilized antibodies or antigen-binding fragments; washing the solid support and assaying for the presence tracer.

In some embodiments, the antibody or antigen-binding fragment are immobilized on the solid support after contacting with the antigen in a reaction chamber. In some embodiments, the antibody or antigen-binding fragment are immobilized on the solid support prior to contacting with the antigen in a reaction chamber. In either case, a tracer may be added to the reaction chamber to detect immobilized binding complexes. In some embodiments, a tracer comprises a detectably labeled secondary antibody directed against the antigen. In some embodiments, the tracer comprises a detectably labeled secondary antibody directed against the primary antibody or antigen-binding fragment. As disclosed herein, the detectable label may be, for example, a radioisotope, a fluorophore, a luminescent molecule, an enzyme, a biotin-moiety, an epitope tag, or a dye molecule. Suitable detectable labels are described herein or may be ascertained from the field.

In some embodiments, it has been found that performing certain immunoassays in low pH buffer leads to more sensitive antigen detection. Accordingly, in some embodiments, a tracer antibody is contacted with a capture antibody in a buffer having a pH in a range of 6.5 to less than 7.75 such that the tracer binds to the capture-antibody-antigen complex. In some embodiments, the buffer pH is about 6.5, 6.6, 6.7, 6.8, 6.9, 7.0, 7.1, 7.2, 7.3, 7.4, 7.5, or 7.6. In other embodiments, the pH is less than 6.5. For example, the pH may be about 5.5, 5.6, 5.7, 5.8, 5.9, 6.0, 6.1, 6.2, 6.3, or 6.4. In certain embodiments, the pH may be less than about 5.5. In certain embodiments, the pH of the buffer used may vary in the different liquid containment regions and at different times during the assay.

It should be appreciated that in any of the assays disclosed herein capture antibodies may be swapped with tracer antibodies.

In some embodiments, an immunoassay that measures the level of fPSA involves contacting fPSA present in the plasma blood sample with a capture antibody specific for fPSA under conditions in which the first capture antibody binds to fPSA, thereby producing a capture-antibody-fPSA complex; and detecting the capture-antibody-fPSA complex using a tracer. In certain embodiments, the capture antibody may be a H117 antibody or fragment thereof (e.g., a F(ab) fragment or a F(ab)₂ fragment). In other embodiments, the capture antibody may be a 5A10, 2C1, 7G1, or 6H10 antibody or fragment thereof (e.g., a F(ab) fragment or a F(ab)₂ fragment). In some embodiments, the tracer comprises a 5A10 or a 2C1 antibody or fragment thereof (e.g., a F(ab) fragment or a F(ab)₂ fragment). In other embodiments, the tracer or capture antibody may be a H117, 2C1, 7G1, 6H10, 5A10, 10-P21A, GWB-258FAA, ab188389, 5G52C10, 1F5H4, 4E9C2, 1D3E6, 1B4A5, M217, M167, M357, or P9054-63 antibody or fragment thereof (e.g., a F(ab) fragment or a F(ab)₂ fragment).

In some embodiments, an immunoassay that measures the level of iPSA involves contacting iPSA present in the plasma blood sample with a capture antibody specific for free PSA, which includes iPSA and nicked PSA, under conditions in which the second capture antibody binds at least to iPSA, thereby producing a capture-antibody-iPSA complex and detecting the capture-antibody-iPSA complex using a second tracer. In some embodiments, the tracer comprises a 4D4 antibody. In some embodiments, the capture antibody is a 5A10 antibody or fragment thereof (e.g., a F(ab) fragment or a F(ab)₂ fragment). In other embodiments, the capture antibody may be 7G1, 2H11, 6H10, or 5E4 antibody or fragment thereof (e.g., a F(ab) fragment or a F(ab)₂ fragment). In certain embodiments, the tracer or capture antibody may be a 4D4, 5A10, 7G1, 2H11, 6H10, 5E4, antibody or fragment thereof (e.g., a F(ab) fragment or a F(ab)₂ fragment) or the antibody from a Human Prostate Specific Antigen (PSA) ELISA Kit from Innovative Research. In one embodiment, the capture antibody is a 5A10 antibody or fragment thereof (e.g., a F(ab) fragment or a F(ab)₂ fragment) and the tracer antibody is a 4D4 antibody or fragment thereof (e.g., a F(ab) fragment or a F(ab)₂ fragment). In another embodiment, the capture antibody is a 4D4 antibody or fragment thereof (e.g., a F(ab) fragment or a F(ab)₂ fragment) and the tracer antibody is a 5A10 antibody or fragment thereof (e.g., a F(ab) fragment or a F(ab)₂ fragment). In certain embodiments, iPSA is not measured in the immunoassay.

In some embodiments, an immunoassay that measures the level of tPSA involves contacting tPSA present in the plasma blood sample with a capture antibody specific for tPSA under conditions in which the third capture antibody binds to tPSA, thereby producing a capture-antibody-tPSA complex; and detecting the capture-antibody-tPSA complex using a third tracer. In some embodiments, the tracer comprises an H50 antibody or fragment thereof (e.g., a F(ab) fragment or a F(ab)₂ fragment). In some embodiments, the tracer comprises a 2E9, 5E4, H117, 6H10, or 2C1 antibody or fragment thereof (e.g., a F(ab) fragment or a F(ab)₂ fragment). In some embodiments, the capture antibody is a H117 antibody. In other embodiments, the capture antibody is a 2C1, 7G1, 2H11, 5E4 or 6H10 antibody or fragment thereof (e.g., a F(ab) fragment or a F(ab)₂ fragment). In other embodiments, the capture or tracer antibody may be an H50, 2E9, 5E4, H117, 6H10, 2C1, 7G1, 2H11, 5A11E9, 251698, 5A11, ab403, ab2218, ab188388, 181823, SPM352, Catalog#10771-H08H, GWB-36F98B, or 214 antibody or fragment thereof (e.g., a F(ab) fragment or a F(ab)₂ fragment).

In some embodiments, an immunoassay that measures the level of hK2 involves contacting PSA in the plasma blood sample with blocking antibodies specific for PSA; contacting hK2 present in the plasma blood sample with a fourth capture antibody specific for hK2 under conditions in which the fourth capture antibody binds to hK2, thereby producing a capture-antibody-hK2 complex; and detecting the capture-antibody-hK2 complex using a fourth tracer. In some embodiments, an immunoassay that measures the level of hK2 involves contacting PSA in the plasma blood sample without using blocking antibodies; contacting hK2 present in the plasma blood sample with a fourth capture antibody specific for hK2 under conditions in which the fourth capture antibody binds to hK2, thereby producing a capture-antibody-hK2 complex; and detecting the capture-antibody-hK2 complex using a fourth tracer. In some embodiments, the tracer comprises a 7G1 antibody or fragment thereof (e.g., a F(ab) fragment or a F(ab)₂ fragment). In other embodiments, the tracer antibody may be 2C1, 5A10, 11B6, 7D7, or 2H11. In some embodiments, the capture antibody is a 6H10 antibody or fragment thereof (e.g., a F(ab) fragment or a F(ab)₂ fragment). In certain embodiments, the capture antibody is a 6H10 F(ab)₂. In other embodiments, the capture antibody may be 2E9, 5E4, H117, 7D7, 11B6, or 6H10. In some embodiments, a blocking antibody is used. In certain embodiments, the blocking antibody comprise a 5H7 antibody, a 5H6 antibody, and/or a 2E9 antibody or fragment thereof. In other embodiments, no blocking antibodies are used. In other embodiments, the capture or tracer antibody may be an 7G1, 2C1, 5A10, 11B6, 7D7, 2H11, 2E9, 5E4, H117, 6H10, orb69302, 2867, 3D3, H.738.7, 10R-4360, sc-130358 (1A7), HK2 antibody from MyBiosource, Inc., or OTI4C5 antibody or fragment thereof (e.g., a F(ab) or F(ab)₂ fragment). In one embodiment, the capture antibody is a 6H10 antibody or fragment thereof (e.g., a F(ab) fragment or a F(ab)₂ fragment) and the tracer antibody is a 7G1 antibody or fragment thereof (e.g., a F(ab) fragment or a F(ab)₂ fragment). In another embodiment, the capture antibody is a 7G1 antibody or fragment thereof (e.g., a F(ab) fragment or a F(ab)₂ fragment) and the tracer antibody is a 6H10 antibody or fragment thereof (e.g., a F(ab) fragment or a F(ab)₂ fragment).

In some embodiments, an immunoassay that measures the level of MSMB involves contacting MSMB present in the plasma blood sample with a capture antibody specific for MSMB under conditions in which the third capture antibody binds to MSMB, thereby producing a capture-antibody-MSMB complex; and detecting the capture-antibody-MSMB complex using a third tracer.

In some embodiments, the capture or tracer antibody may be an 6C7, ab128897, ab19070, ab180479 (2E7), OTI6C7, GTX84083, LS-C336888, YPSP-3, M08, or monoclonal anti-MSMB produced in mouse from Sigma-Aldrich, Inc. antibody or fragment thereof (e.g., a F(ab) fragment or a F(ab)₂ fragment).

In some embodiments, an immunoassay that measures the level of MIC-1 involves contacting MIC-1 present in the plasma blood sample with a capture antibody specific for MIC-1 under conditions in which the third capture antibody binds to MIC-1, thereby producing a capture-antibody-MIC-1 complex; and detecting the capture-antibody-MIC-1 complex using a third tracer. In some embodiments, the capture or tracer antibody may be an sc-390305, 4E4, OTI4E4, MIC1-1C3, D2A3, or LS-C173852 antibody or fragment thereof (e.g., a F(ab) fragment or a F(ab)₂ fragment).

Table 1 below lists antibodies and antigen-binding fragments that may be used in the methods disclosed herein and their corresponding antigens or epitopes. Additional antibodies for measuring intact PSA may be found in, for example, Pauliina Nurmikko et al., Clinical Chemistry October 2000, 46 (10) 1610-1618, or in any of the references and sources cited in Table 1, the entire contents of each of which is incorporated herein by reference for their teachings of antibodies.

TABLE 1
Antibodies and Corresponding Epitopes or Antigens

| Antibody Name | Epitope/Antigen | Reference or Source |
|---|---|---|
| 2E9 | amino acids 79-93 and/or 80-91 | Lilja et al. 1991. Prostate-Specific |
| | of PSA protein | Antigen in Serum Occurs |
| | | Predominantly in Complex with |
| | | alpha-1-Antichymotrypsin. Clin |
| | | Chem. 37(9), 1618-1625. |
| | | Piironen, et al. Determination and |
| | | analysis of antigenic epitopes of |
| | | prostate specific antigen (PSA) and |
| | | human glandular kallikrein 2 (hK2) |
| | | using synthetic peptides and |
| | | computer modeling. Protein |
| | | Science (1998), 7: 259-269 |
| 5F7 | | Nurmikko et al. 2000. Production |
| | | and Characterization of Novel Anti- |
| | | Prostate-specific Antigen (PSA) |
| | | Monoclonal Antibodies That Do |
| | | Not Detect Internally Cleaved |
| | | Lys145-Lys146 Inactive PSA. Clin |
| | | Chem. 46(10): 1610-1618. |
| 5H6 | amino acids 225-237 of PSA | Nurmikko et al. 2000. Supra |
| | protein | |
| 7G1 | recognizes PSA, PSA-ACT, | Nurmikko et al. 2000. Supra |
| | hK2, and hK2-ACT with equal | |
| | affinities | |
| Fab 5A10 | amino acids 75-89, 80-94 | Eriksson et al. 2000. Dual-label |
| | and/or 82-39 of PSA protein | time-resolved immunofluorometric |
| | | assay of free and total Prostate- |
| | | specific Antigen Based on |
| | | Recombinant Fab Fragments. Clin |
| | | Chem 46(5), 658-666. Piironen et |
| | | al. Protein; 1998: 7: 259-69. |
| 4D4 | amino acids 130-144 of PSA | U.S. Pat. No. 7,872,104 |
| | protein | |
| 5A10 | amino acids 75-89, 80-94 | U.S. Pat. No. 5,939,533, |
| | and/or 82-39 of PSA protein | European Collection of Animal Cell |
| | | Cultures Accession number 93091201. |
| | | Piironen et al., 1998 |
| 2C1 | Free PSA and PSA-ACT | Pettersson K, Piironen T, Seppälä |
| | (amino acids 50-64 and 55-69 | M, Liukkonen L, Christensson A, |
| | of the PSA protein), hK2 | Matikainen M T, Suonpää M, et al. |
| | | Free and complexed prostate- |
| | | specific antigen (PSA): In vitro |
| | | stability, epitope map, and development |
| | | of immunofluorometric assays for |
| | | specific and sensitive detection of |
| | | free PSA and PSA-alpha1- |
| | | antichymotrypsin complex. Clin |
| | | Chem 1995; 41: 1480-8; Rajakoski, |
| | | Piironen, Pettersson, Lo, & Karp; |
| | | Epitope mapping of human prostate- |
| | | specific antigen and glandular |
| | | kallikrein expressed in insect cells. |
| | | Prostate Cancer Prostatic Dis. 1997; |
| | | 1(1): 16-20; Piironen et al., 1998 |
| 3C1 | Free PSA and PSA-ACT, hK2 | Pettersson et al., 1995; Piironen et |
| | | al., 1998 |
| 4H5 | Free PSA and PSA-ACT, hK2 | Pettersson et al., 1995; Piironen et |
| | | al., 1998 |
| 5F11 | Free PSA and PSA-ACT, hK2 | Piironen et al., 1998 |
| F5 | Free PSA and PSA-ACT, hK2 | Piironen et al., 1998 |
| 2H11 | Free PSA and PSA-ACT | Pettersson et al., 1995; Piironen et |
| | (regions of the PSA protein that | al., 1998; Rajakoski, Piironen, |
| | include amino acids 53-64 and | Pettersson, Lo, & Karp 1997; |
| | 151-164) | Stenman et al., Summary report of |
| | | the TD-3 workshop: characterization |
| | | of 83 antibodies against prostate- |
| | | specific antigen. 1999; Tumour |
| | | Biol. 1999; 20 Suppl 1: 1-12. |
| 5E4 | PSA (amino acids 3 to 10, 140- | Leinonen, Wu, & Stenman, Epitope |
| | 142), hK2 | mapping of antibodies against |
| | | prostate-specific antigen with use of |
| | | peptide libraries. 2002 (Dec) Clin |
| | | Chem.; 48(12): 2208-16.; Wang et |
| | | al., Western blotting analysis of |
| | | antibodies to prostate-specific |
| | | antigen: specificities for prostate- |
| | | specific antigen and prostate- |
| | | specific antigen fragments. 1999; |
| | | Tumour Biol. 1999; 20 Suppl 1: 79- |
| | | 85.; Leinonen J, Leinimaa M, Zhang |
| | | W-M, Piironen T, Pettersson K, |
| | | Lilja H, Dowell B, et al. Reactivity |
| | | of Anti-PSA Monoclonal |
| | | Antibodies with Recombinant |
| | | Human Kallikrein-2. Tumor Biol |
| | | 1999; 20 Suppl 1: 35-7; Stenman et |
| | | al., 1999 |
| Kallikrein 3/PSA | PSA protein (measuring Total | Novus Biologicals |
| Antibody (5A11E9) | PSA) | |
| PSA Antibody (Cat. | PSA protein (measuring Total | Abbiotec, LLC. |
| No. 251698) | PSA) | |
| PSA (5A11) Antibody | PSA protein (measuring Total | Abbiotec, LLC. |
| | PSA) | |
| Anti-Prostate Specific | PSA protein (measuring Total | Abcam, PLC. |
| Antigen antibody | PSA) | |
| [8301](ab403) | | |
| Anti-Prostate Specific | PSA protein (measuring Total | Abcam, PLC. |
| Antigen antibody | PSA) | |
| [A67-B/E3] (ab2218) | | |
| Human Total Prostate | PSA protein (measuring Total | Abcam, PLC. |
| Specific Antigen | PSA) | |
| ELISA Kit (ab188388) | | |
| Kallikrein 3/PSA | PSA protein (measuring Total | Novus Biologicals |
| Antibody (181823) | PSA) | |
| [Unconjugated] | | |
| Kallikrein 3/PSA | PSA protein (measuring Total | Novus Biologicals |
| Antibody (SPM352) | PSA) | |
| Recombinant Human | PSA protein (measuring Total | Sino Biological, Inc. |
| KLK3/Kallikrein 3 | PSA) | |
| protein | | |
| (Catalog#10771- | | |
| H08H) | | |
| PSA Total Antibody | PSA protein (measuring Total | GenWay Biotech, Inc. |
| (GWB-36F98B) | PSA) | |
| Prostate Specific | PSA protein (measuring Total | Bio-Rad Laboratories, Inc. |
| Antigen Antibody \| | PSA) | |
| 214 | | |
| PSA antibody (10- | PSA protein (measuring Free | Fitzgerald Industries International |
| P21A) | PSA) | |
| PSA Free Antibody | PSA protein (measuring Free | GenWay Biotech, Inc. |
| (GWB-258FAA) | PSA) | |
| Human Free Prostate | PSA protein (measuring Free | Abcam, PLC. |
| Specific Antigen | PSA) | |
| ELISA Kit (ab188389) | | |
| PSA, free, Monoclonal | PSA protein (measuring Free | Full Moon Biosystems |
| Antibody, 5G52C10 | PSA) | |
| (Cat. No. MP017) | | |
| PSA, free, Monoclonal | PSA protein (measuring Free | Full Moon Biosystems |
| Antibody, 1F5H4 | PSA) | |
| Human free PSA | PSA protein (measuring Free | GenScript |
| Antibody, mAB, | PSA) | |
| Mouse (4E9C2, | | |
| 1D3E6 and 1B4A5) | | |
| Human Free PSA | PSA protein (measuring Free | CalBioreagents |
| (M217, M167, M357) | PSA) | |
| P9054-63 Mouse Anti- | PSA protein (measuring Free | United States Biological |
| Prostate Specific | PSA) | |
| Antigen, Free (Free | | |
| PSA) | | |
| Human Prostate | PSA protein (measuring Intact | Innovative Research |
| Specific Antigen | PSA) | |
| (PSA) ELISA Kit | | |
| F(ab)₂ 6H10 | 20-fold higher affinity for hK2 | Becker et al. 2000. Sensitive and |
| | when compared to affinity for | Specific Immunodetection of |
| | PSA; undetectable binding for | Human Glandular Kallikrein 2 in |
| | PSA when blockers 2E9, 5H6, and | Serum. Clin Chem. 46(2), 198-206; |
| | 5F7 are added during capture step | Väisänen et al., Development of |
| | | sensitive immunoassays for free and |
| | | total human glandular kallikrein 2. |
| | | Clin Chem. 2004 September; 50(9): 1607-17. |
| 7D7 | selective for free hK2, with | Väisänen et al., 2004; |
| | recognition toward hK2-PCI | US20160369009 |
| | (protein C inhibitor), hK2-PAI- | |
| | 1 (plasmiogen activator | |
| | inhibitor-1), and hK2-AT | |
| | (antitrypsin) at 7%, 4%, 5%, | |
| | and 24% that of free hK2; | |
| | affinity constant for hK2 7.4 × | |
| | 10\hat{ }8 L/mol | |
| 11B6 | selective for free hK2, with | Väisänen et al., 2004; U.S. Pat. No. 9,345,782 |
| | recognition toward hK2-PCI, | |
| | hK2-PAI-l, and hK2-AT at 2%, | |
| | 31%, 26%, and 35% that of free | |
| | hK2; affinity constant for hK2 | |
| | at 7.9 × 10\hat{ }8 L/mol | |
| 11B6 (recombinant) | see 11B6 above | Creative Biolabs |
| TAB-0622CL | | |
| 11B6 scFv | see 11B6 above | Creative Biolabs |
| TAB-0622CL-S(P) | | |
| HB6Fab | see 11B6 above | Creative Biolabs |
| TAB-0622CL-F(E) | | |
| EPR6676 (ab124899) | hK2 | Abcam |
| | Approximate K_{D} 8.78 × 10\hat{ }−11 | |
| 6B7 (ab40749) | hK2 | Abcam |
| | No detectable binding to PSA | W02003029427A2 |
| | | Fisher T L, et al. Prostate; |
| | | 2002; 51: 153-65. |
| 3C5; Catalog # | hK2 | Abnova |
| H00003817-M03 | | |
| 5D6; Catalog # LS- | hK2 | LSBio |
| C336461 | | |
| LS-C308674 | hK2 | LSBio |
| Clone 426723 | hK2 | R&D Systems |
| 3E6 | hK2 | W02003029427A2; |
| | No detectable binding to PSA | University Of Rochester |
| | | Fisher T L, et al. Prostate; |
| | | 2002; 51: 153-65. |
| 1F8 | hK2 | W02003029427A2; |
| | No detectable binding to PSA | University Of Rochester |
| | | Fisher T L, et al. Prostate; |
| | | 2002; 51: 153-65. |
| 3C7 | hK2 | W02003029427A2; |
| | | University Of Rochester |
| | | Fisher T L, et al. Generation of |
| | | monoclonal antibodies specific for |
| | | human kallikrein 2 (hK2) using |
| | | hK2-expressing tumors. Prostate; |
| | | 2002; 51: 153-65. |
| 11C4 | hK2 | W02003029427A2; |
| | | University Of Rochester |
| | | Fisher T L, et al. Prostate; |
| | | 2002; 51: 153-65. |
| 9B4 | hK2 | W02003029427A2; |
| | | University Of Rochester |
| | | Fisher T L, et al. Prostate; |
| | | 2002; 51: 153-65. |
| 2D3 | hK2 and PSA; higher affinity | W02003029427A2; |
| | for hK2 but also binds PSA | University Of Rochester |
| | | Fisher T L, et al. Prostate; |
| | | 2002; 51: 153-65. |
| OTI5D6 (formerly 5D6) | hK2 | Acris |
| TA802077 | | |
| Kallikrein 2 Antibody | hK2 | Invitrogen |
| (PA5-27208) | | |
| Kallikrein 2 Antibody | hK2 | Invitrogen |
| (PA5-18674) | | |
| Kallikrein 2 Antibody | hK2 | Invitrogen |
| (PA5-13342) | | |
| 8311 | strong reactivity to hK2 (also | Leinonen J, et al. Reactivity of anti- |
| | binds free and complexed PSA) | PSA monoclonal antibodies with |
| | | recombinant human kallikrein-2. |
| | | Tumor Biol; 1999; 20: 35-7. |
| 11E5C6 | weak reactivity to hK2 (also | Leinonen J, et al. Tumor Biol; |
| | binds free and complexed PSA) | 1999; 20: 35-7. |
| 12C11C3 | strong reactivity to hK2 (also | Leinonen J, et al. Tumor Biol; |
| | binds free and complexed PSA) | 1999; 20: 35-7. |
| 1C5 | weak reactivity to hK2 (also | Leinonen J, et al. Tumor Biol; |
| | binds free and complexed PSA) | 1999; 20: 35-7. |
| 7P401 | strong reactivity to hK2 (also | Leinonen J, et al. Tumor Biol; |
| | binds free and complexed PSA) | 1999; 20: 35-7. |
| 92-283 | weak reactivity to hK2 (also | Leinonen J, et al. Tumor Biol; |
| | binds free and complexed PSA) | 1999; 20: 35-7. |
| 94R48 | weak reactivity to hK2 (also | Leinonen J, et al. Tumor Biol; |
| | binds free and complexed PSA) | 1999; 20: 35-7. |
| 9C5 | weak reactivity to hK2 (also | Leinonen J, et al. Tumor Biol; |
| | binds free and complexed PSA) | 1999; 20: 35-7. |
| E72 | weak reactivity to hK2 (also | Leinonen J, et al. Tumor Biol; |
| | binds free and complexed PSA) | 1999; 20: 35-7. |
| E74 | strong reactivity to hK2 (also | Leinonen J, et al. Tumor Biol; |
| | binds free and complexed PSA) | 1999; 20: 35-7. |
| E87 | strong reactivity to hK2 (also | Leinonen J, et al. Tumor Biol; |
| | binds free and complexed PSA) | 1999; 20: 35-7. |
| E89 | weak reactivity to hK2 (also | Leinonen J, et al. Tumor Biol; |
| | binds free and complexed PSA) | 1999; 20: 35-7. |
| M704 | strong reactivity to hK2 (also | Leinonen J, et al. Tumor Biol; |
| | binds free and complexed PSA) | 1999; 20: 35-7. |
| PCS#4 | strong reactivity to hK2 (also | Leinonen J, et al. Tumor Biol; |
| | binds free and complexed PSA) | 1999; 20: 35-7. |
| PSA399 | weak reactivity to hK2 (also | Leinonen J, et al. Tumor Biol; |
| | binds free and complexed PSA) | 1999; 20: 35-7. |
| PSA66 | strong reactivity to hK2 (also | Leinonen J, et al. Tumor Biol; |
| | binds free and complexed PSA) | 1999; 20: 35-7. Piironen et al., 1998. |
| | | Nilsson et al., Antigenic |
| | | determinants of prostate-specific |
| | | antigen (PSA) and development of |
| | | assays specific for different forms of |
| | | PSA. Br J Cancer Nature; 1997; |
| | | 75: 789-97 |
| H117 | strong reactivity to hK2 (also | U.S. Pat. No. 5,672,480 |
| | binds free and complexed PSA) | Leinonen J, et al. Tumor Biol; |
| | | 1999; 20: 35-7.; Piironen et al., 1998 |
| H50 | strong reactivity to hK2 (also | Leinonen J, et al. Tumor Biol; |
| | binds free and complexed PSA) | 1999; 20: 35-7. |
| H50 | binds both hK2 and free and | Piironen et al., 1998 |
| | complexed PSA | |
| H179 | binds both hK2 and free and | Piironen et al., 1998 |
| | complexed PSA | |
| MIC1 Antibody (D-7): | MIC1 protein | Santa Cruz Biotechnology, Inc. |
| sc-390305 | | |
| Mic1 Antibody (4E4) | MIC1 protein | Novus Biologicals |
| Anti-C18orf8 | MIC1 protein | OriGene Technologies, Inc. |
| TRUEMAB Antibody | | |
| Clone OTI4E4 | | |
| Oval Cell Marker | MIC1 protein | Thermo Fisher Scientific, Inc. |
| Antibody (MIC1-1C3) | | |
| Mic-1 (D2A3) Rabbit | MIC1 protein | Cell Signaling Technology, Inc. |
| mAb #8479 | | |
| Anti-C18orf8/MIC1; | MIC1 protein | LifeSpan BioSciences, Inc. |
| Antibody (clone 4E4) | | |
| LS-C173852 | | |
| Prostate Secretory | MSMB protein | Novus Biologicals |
| Protein/PSP Antibody | | |
| (6C7) | | |
| Anti-Prostate | MSMB protein | Abcam, PLC. |
| Secretory Protein/PSP | | |
| antibody [EPR7345] | | |
| (ab128897) | | |
| Anti-Prostate | MSMB protein | Abcam, PLC. |
| Secretory Protein/PSP | | |
| antibody [YPSP-1] | | |
| (ab19070) | | |
| Anti-Prostate | MSMB protein | Abcam, PLC. |
| Secretory Protein/PSP | | |
| antibody [2E7] | | |
| (ab180479) | | |
| Anti-MSMB | MSMB protein | OriGene Technologies, Inc. |
| TRUEMAB Antibody | | |
| Clone OTI6C7 | | |
| GTX84083 MSMB | MSMB protein | GeneTex Inc |
| antibody [6C7] | | |
| Anti-MSMB/MSP | MSMB protein | LifeSpan BioSciences, Inc. |
| Antibody (clone 4C1) | | |
| LS-C336888 | | |
| MSMB monoclonal | MSMB protein | Abnova Corporation |
| antibody, clone YPSP-3 | | |
| Monoclonal Anti- | MSMB protein | Sigma-Aldrich, Inc. |
| MSMB antibody | | |
| produced in mouse | | |
| MSMB monoclonal | MSMB protein | Abnova Corporation |
| antibody (M08), clone | | |
| 3B11 | | |

Reagents can be stored in or on a chip for various amounts of time. For example, a reagent may be stored for longer than 1 hour, longer than 6 hours, longer than 12 hours, longer than 1 day, longer than 1 week, longer than 1 month, longer than 3 months, longer than 6 months, longer than 1 year, or longer than 2 years. Optionally, the chip may be treated in a suitable manner in order to prolong storage. For instance, chips having stored reagents contained therein may be vacuum sealed, stored in a dark environment, and/or stored at low temperatures (e.g., below 4 degrees C. or 0 degrees C.). The length of storage depends on one or more factors such as the particular reagents used, the form of the stored reagents (e.g., wet or dry), the dimensions and materials used to form the substrate and cover layer(s), the method of adhering the substrate and cover layer(s), and how the chip is treated or stored as a whole. Storing of a reagent (e.g., a liquid or dry reagent) on a solid support material may involve covering and/or sealing the chip prior to use or during packaging.

The quantification of presence or concentration of any of the proteins (markers) discussed herein can be performed in various ways including, but not limited to, immunoprecipitation assays, immuno-fluorescence assays, radio-immuno-assays, and mass spectrometry using matrix-assisted laser desorption/ionization (MALDI). Another method for determining the presence of any of the proteins (markers) discussed herein comprises the use of enzyme linked immunosorbent assays (ELISA) which use antibodies and a calibration curve to assess the presence and/or concentration of one or more selected markers. See, for example “Association between saliva PSA and serum PSA in conditions with prostate adenocarcinoma.” by Shiiki N et al., published in Biomarkers. 2011 September; 16(6):498-503, which is hereby incorporated by reference herein. Another method for determining the presence or concentration of one or more of the proteins (markers) discussed herein is the use of a microarray assay. A typical microarray assay comprises a flat glass slide onto which a plurality of different capture reagents (typically one or more antibodies) each selected to specifically capture one type of biomarker (protein) is attached in non-overlapping areas on one side of the slide. A sample (e.g., a blood sample such as a blood plasma sample) is allowed to contact an area (e.g., one or more liquid containment regions) where the capture reagents are located. Subsequently, the area where the capture reagents are located is subjected to one or more washes. Next, one or more detection reagents are added to the area where the capture reagents are located (which now also potentially comprises bound proteins (markers) from the sample). The detection reagents selected for use in this assay should be capable of (i) binding to the protein (marker) presented on the glass slide and (ii) producing a detectable signal (e.g., a fluorescent signal).

### SNP Analysis

Additional genetic profile information may also be used in the assays and methods disclosed herein. One example of genetic profile information that may be gathered and used is SNP analysis. The quantification of SNP data through the analysis of a biological sample typically involves MALDI mass spectrometry analysis based on allele-specific primer extensions, even though other methods are equally applicable. This applies to any type of genetic status, i.e., any combination of SNPs related to prostate cancer or prostate cancer risk factors. The quantification of SNP data may be performed on the same chip as any of the other assays described herein. The quantification of SNP data may be performed on one or more additional chips (i.e., the quantification of SNP data may be performed on a chip that does not comprise any of the other assays described herein). In some embodiments, no additional genetic profile information is used in or with the assays and methods described herein. In certain embodiments, no SNP analysis is used in or with the assays and methods described herein.

Certain SNPs related to solid tumor cancer are SNPs known to be related to prostate cancer, which may include, but are not limited to: rs12621278 (Chromosome 2, locus 2q31.1), rs9364554 (Chromosome 6, locus 6q25.3), rs10486567 (Chromosome 7, locus 7pl 5.2), rs6465657 (Chromosome 7, locus 7q21.3), rs2928679 (Chromosome 8, locus 8pl 1), rs6983561 (Chromosome 8, locus 8q24.21), rs16901979 (Chromosome 8, locus 8q24.21), rs16902094 (Chromosome 8, locus 8q24.21), rs12418451 (Chromosome 11, locus 11 q 13.2), rs4430796 (Chromosome 17, locus 17q 12), rs11649743 (Chromosome 17, locus 17q12), rs2735839 (Chromosome 19, locus 19q13.33), rs9623117 (Chromosome 22, locus 22q13.1), and rs138213197 (Chromosome 17, locus 17q21). SNPs related to prostate cancer may also include, but are not limited to: rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, and rs2405942. Other SNPs related to prostate cancer may include rs138213197 as described in the report “Germline mutations in HOXB13 and prostate-cancer risk.” by Ewing C M et al., N Engl J Med. 2012 Jan. 12; 366(2):141-9, 11OOdelC (22ql2.1) and I157T (22ql2.1) as described in “A novel founder CHEK2 mutation is associated with increased prostate cancer risk.” by Cybulski et al., Cancer Res. 2004 Apr. 15; 64(8):2677-9, and 657del5 (8q21) as described in “NBS1 is a prostate cancer susceptibility gene” by Cybulski et al., Cancer Res. 2004 Feb. 15; 64(4): 1215-9. Each of the foregoing references is incorporated herein by reference in its entirety). Additional SNPs relevant to prostate cancer may include, but are not limited to SNPs related to the concentration or expression level of relevant biomarkers such as Prostate-specific antigen (PSA) in either free form or complexed form, total PSA (tPSA), intact PSA (iPSA), human kallikrein 2 (hK2), early prostate cancer antigen (EPCA), Macrophage Inhibitory Cytokine 1 (also known as MIC-1 or GDF-15), MSMB (also known as Prostate Secretory Protein, PSP94, and beta-microseminoprotein), human prostatic acid phosphatase (PAP), glutathione S-transferase π (GSTP1), a-methylacyl coenzyme A racemase (AMACR). Further SNPs relevant to prostate cancer may include, but are not limited to SNPs related to the concentration or expression level of relevant biomarkers such as 14-3-3 (YWHAG), antigen receptor isoforms (e.g., WT, T877A, and/or 0CAG), antithrombin-III, arginase-2 mitochondrial (ARG2), ATP-synthase-β-chain (ATP5B), Bax (Bcl-2-associated X protein), B-tubulin (TUBB), caveolin-1, caveolin-2, carnitine palmitoyltransferase 2 (CPT2), cellular retinoic acid-binding protein 2 (CRABP2), clusterin (CLU), coatomer protein complex, subunit alpha (COPA), complement C3, complement C4-B, complement C4a truncated form (C4a des-Arg), creatine kinase-β-chain (CKB), cytokeratin 7 (KRT7), cytokeratin 8 (KRT8), cytokeratin 18 (KRT18), desmin (DES), dimethylargininedimethylaminohydrolase 1 (DDAH1), enhancer of zeste homolog 2 (EZH2), enoyl CoA-hydrase, EPLIN (epithelial protein lost in neoplasm), eukaryotic initiation factor 4A-III (eIF4A3), ezrin (EZR), fatty acid-binding protein, epidermal (FABP5), filamin-A (FLNA), FK506-binding protein 4 (FKBP4), growth differentiation factor 15 (GDF15), haptoglobin, hemopexin, HER2, HERS, HSP60, HSP70, HSP71, inorganic pyrophosphatase 2 (PPA2), inosine monophosphate dehydrogenase II (IMPDH2), keratin-II (KRT2), lamin A (LMNA), metaxin 2 (MTX2) increased, Metalloproteinase inhibitor-1 (TIMP1), methylcrotonoyl coenzyme A carboxylase 2 (beta) (MCCC2), MSK1/2 (mitogen- and stress-activated protein kinase 1 and 2 protein kinase), NAAA (N-acylethanolamine acid amidase), nucleoside diphosphate kinase 1 (NDPK1), periostin (POSTN), peroxiredoxins (e.g., PRDX3 and PRDX4), pigment epithelium-derived factor (PEDF), pro-NPY, PPP1CB (serine/threonine-protein phosphatase PP1β), prohibitin (PHB), protein C inhibitor-N-terminal fragment, protein disulfide isomerase (P4HB), prothrombin, PSMB6 (proteasome subunit, beta type, 6), PTEN, PTK7 (Tyrosine kinase 7), secernin-1, serum amyloid A-1 protein, SLP2, SM22, STAT3, Smac/Diablo, TFG (TRK-fused gene), TTR (Transthyretin), tumor necrosis factor receptor-associated Protein 1 (TRAP1), UBE2N (Ubiquitin-conjugating enzyme E2N), vinculin, and/or zinc-alpha2-glycoprotein (ZAG).

Additional SNPs relevant to prostate cancer may include, but are not limited to: rs3213764, rs1354774, rs2736098, rs401681, rs10788160, and rs11067228 (related to the expression level of PSA); rs3213764 and rs1354774 (related to the expression level of free PSA); rs1363120, rs888663, rs1227732, and rs1054564 (related to the expression level of the inflammation cytokine biomarker MIC-1); and rs3817334, rs10767664, rs2241423, rs7359397, rs7190603, rs571312, rs29941, rs2287019, rs2815752, rs713586, rs2867125, rs9816226, rs10938397, and rs1558902 (related to the BMI of an individual). Additional SNPs relevant to prostate cancer may include, but are not limited to those SNPs discussed in “Contribution of 32 GWAS-identified common variants to severe obesity in European adults referred for bariatric surgery” by Magi et al., PLoS One (2013) Aug. 7; 8(8):e70735 (which is incorporated by reference herein).

Any set or subset of the SNPs listed herein is suitable for use in the assays or methods of the instant disclosure. Other additional SNPs known in the art or disclosed herein may also be used in the methods or assays of the instant disclosure. The SNPs disclosed (all or any subset of the SNP listed herein) may be bound to one or more solid supports or chips as disclosed herein or known in the art. As an example, about 1, 2, 3, 4, 5, 6, 7, 8, 9, 10, 15, 20, 25, 30, 35, 40, 45, 50, 55, 60, 65, 70, 75, 80, 85, 90, 95, 100, or more than 100 SNPs may be used in the assays and/or methods disclosed herein. As another example, about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of the SNPs listed herein may be used in the assays and/or methods disclosed herein.

In one aspect, when one or more SNPs are used in a method as defined herein in the context of prostate cancer, the data regarding the one or more SNPs is combined with one or more of the biomarkers selected from the group consisting of: PSA, free PSA, intact PSA, hK2 (total hK2 or free hK2), MIC-1 and MSMB, such as 1, 2, 3, 4, 5, 6, 7, or 8 members of this group. In another aspect, when one or more SNPs are used in a method as defined herein in the context of prostate cancer, the data regarding the one or more SNPs is combined with one or more of the biomarkers selected from the group consisting of: PSA, free PSA, hK2 (total hK2 or free hK2), MIC-1 and MSMB, such as 1, 2, 3, 4, 5, 6, 7, or 8 members of this group.

Group 1

SNPs which are optionally included are selected from the group consisting of: rs138213197, rs7818556, rs6983267, rs10993994, rs12793759, rs16901979, rs9911515, rs1016343, rs7106762, rs6579002, rs16860513, rs5945619, rs16902094, rs10896437, rs651164, rs7679673, rs13265330, rs2047408, rs10107982, rs620861, rs9297746, rs1992833, rs7213769, rs2710647, rs888507, rs17021918, rs12500426, rs2028900, rs7102758, rs16901922, rs6062509, rs2659051, rs17832285, rs12543663, rs4699312, rs11091768, rs3120137, rs6794467, rs10086908, rs7141529, rs2315654, rs12151618, rs747745, rs1009, rs2132276, rs2735839, rs11568818, rs684232, rs9364554, rs9830294, rs2660753, rs10807843, rs1933488, rs17467139, rs12947919, rs721048, rs385894, rs2331780, rs1894292, rs2107131, rs6545962, rs11649743, rs758643, rs2297434, rs902774, rs2647262, rs17224342, rs5918762, rs11672691, rs17138478, rs3019779, rs1873555, rs9457937, rs2838053, rs12946864, rs12475433, rs3765065, rs2018334, rs3771570, rs4871779, rs10875943, rs11601037, rs6489721, rs11168936, rs9297756, rs11900952, rs6569371, rs7752029, rs5934705, rs3745233, rs1482679, rs749264, rs6625760, rs5978944, rs2366711, rs5935063, rs10199796, rs2473057, rs4925094, and rs3096702, or a subset thereof

Group 2

Further SNPs which are optionally included are selected from the group consisting of: rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197, or a subset thereof.

Group 3

Further SNPs which are optionally included are selected from the group consisting of: rs12490248, rs4245739, rs10094059, rs306801, rs2823118, rs2025645, rs9359428, rs10178804, rs6090461, rs2270785, rs16901841, rs2465796, rs17256058, rs16849146, rs2269640, rs8044335, rs6530238, rs712242, rs9267911, rs11134144, rs12880777, rs7090755, rs132774, rs17779822, rs398146, rs4844228, rs4237185, rs7125415, rs1439024, rs6770955, rs11253002, rs4822763, rs2162185, rs12640320, rs5945637, rs3818714, rs6762443, rs10508678, rs2272668, rs2227270, rs6437715, rs3759129, rs1891158, rs7358335, rs12988652, rs3796547 rs7234917, rs6509345, rs966304, rs1515542, rs11631109, rs871688, rs4382847, rs9972541, rs13113975, rs4119478, rs1380862, rs7529518, rs785437, rs1140809, rs4830488, rs10458360, rs2738571, rs11634741, rs1950198, rs539357, rs16887736, rs7658048, rs11222496, rs2207790, rs12506850, rs4512641, rs2813532, rs6934898, rs582598, rs10191478, rs10486562, rs17395631, rs7525167, rs12637074, rs10887926, rs7485441, rs1944047, rs7178085, rs17318620, rs10489871, rs2691274, rs6962297, rs1827611, rs4806120, rs7164364, rs2293710, rs13017302, rs4570588, rs2386841, rs40485, rs524908, rs10795841, rs4273907, rs12612891, rs10496470, rs6755901, rs1943821. rs13319878, rs6957416, rs12552397, rs6489794, rs4346531, rs7777631, rs1046011, rs16988279, rs986472, rs10508422, rs9456490, rs1295683, rs2449600, rs7075945, rs9358913, rs1477886, rs753032, rs409558, rs4246742, rs10060513, rs17070292, rs10826398, rs17744022, rs7801918, rs885479, rs1863610, rs3805284, rs10832514, rs2509867, rs2070874, rs2339654, rs12903579, rs11610799, rs2272316, rs6961773, rs2078277, rs17324573, rs6760417, rs2911756, rs12233245, rs896615, rs4760442, rs2087724, rs439378, rs4833103, rs6539333, rs4423250, rs12594014, rs17123359, rs12505546, and rs585197, or a subset thereof.

Group 4

Further SNPs which are optionally included are selected from the group consisting of:

rs582598, rs439378, rs2207790, rs1046011, rs10458360, rs7525167, rs10489871, rs7529518, rs4245739, rs4512641, rs10178804, rs11900952, rs1873555, rs10191478, rs6755901, rs6545962, rs721048, rs2710647, rs12612891, rs2028900, rs1009, rs12233245, rs6760417, rs10496470, rs10199796, rs12475433, rs16860513, rs12151618, rs3765065, rs13017302, rs12988652, rs871688, rs749264, rs3771570, rs4346531, rs6770955, rs12637074, rs2660753, rs13319878, rs6437715, rs2162185, rs1515542, rs2270785, rs9830294, rs1439024, rs6762443, rs888507, rs6794467, rs12490248, rs1477886, rs4833103, rs3796547, rs17779822, rs2366711, rs16849146, rs1894292, rs12640320, rs3805284, rs12500426, rs4699312, rs17021918, rs7679673, rs2047408, rs2647262, rs12506850, rs7658048, rs2078277, rs12505546, rs13113975, rs4246742, rs2736098, rs401681, rs11134144, rs10060513, rs40485, rs2087724, rs1482679, rs16901841, rs1295683, rs2070874, rs7752029, rs2018334, rs9358913, rs1140809, rs409558, rs3096702, rs9267911, rs2025645, rs9359428, rs6569371, rs2813532, rs1933488, rs712242, rs6934898, rs9456490, rs651164, rs3120137, rs9364554, rs9457937, rs10486562, rs10807843, rs7801918, rs6962297, rs2465796, rs6957416, rs7777631, rs2272316, rs6961773, rs2132276, rs13265330, rs16887736, rs2911756, rs2272668, rs2339654, rs1380862, rs9297746, rs12543663, rs10086908, rs16901922, rs1016343, rs17832285, rs16901979, rs4871779, rs10107982, rs16902094, rs620861, rs17467139, rs6983267, rs9297756, rs10094059, rs7818556, rs1992833, rs986472, rs12552397, rs4273907, rs4237185, rs753032, rs11253002, rs2386841, rs10795841, rs10508422, rs7075945, rs10508678, rs539357, rs10826398, rs3818714, rs7090755, rs10993994, rs4382847, rs1891158, rs10887926, rs10788160, rs6579002, rs10832514, rs7358335, rs1944047, rs3019779, rs10896437, rs12793759, rs7106762, rs7102758, rs2449600, rs585197, rs2509867, rs11568818, rs7125415, rs11601037, rs11222496, rs4570588, rs6489721, rs3213764, rs17395631, rs4423250, rs11168936, rs10875943, rs3759129, rs902774, rs1827611, rs4760442, rs11610799, rs6539333, rs11067228, rs7485441, rs6489794, rs4119478, rs17070292, rs2293710, rs17256058, rs1950198, rs2331780, rs7141529, rs12880777, rs17123359, rs785437, rs524908, rs12903579, rs7178085, rs7164364, rs896615, rs11634741, rs9972541, rs12594014, rs11631109, rs1558902, rs8044335, rs2738571, rs885479, rs385894, rs684232, rs4925094, rs17138478, rs11649743, rs2107131, rs7213769, rs12946864, rs306801, rs138213197, rs1863610, rs17224342, rs9911515, rs12947919, rs966304, rs17744022, rs7234917, rs1943821, rs2227270, rs1363120, rs888663, rs1227732, rs1054564, rs4806120, rs11672691, rs758643, rs3745233, rs6509345, rs2659051, rs2735839, rs1354774, rs2691274, rs6090461, rs2297434, rs6062509, rs2315654, rs2823118, rs2838053, rs398146, rs16988279, rs2269640, rs4822763, rs132774, rs747745, rs5978944, rs6530238, rs5934705, rs5935063, rs4830488, rs17318620, rs5945619, rs5945637, rs11091768, rs2473057, rs5918762, rs4844228, rs6625760 and rs17324573, or a subset thereof.

Group 5

Further SNPs which are optionally included are selected from the group consisting of: rs138213197, rs7818556, rs6983267, rs10993994, rs12793759, rs16901979, rs9911515, rs1016343, rs7106762, rs6579002, rs16860513, rs5945619, rs16902094, rs10896437, rs651164, rs7679673, rs13265330, rs2047408, rs10107982, rs620861, rs9297746, rs1992833, rs7213769, rs2710647, rs888507, rs17021918, rs12500426, rs2028900, rs7102758, rs16901922, rs6062509, rs2659051, rs17832285, rs12543663, rs4699312, rs11091768, rs3120137, rs6794467, rs10086908, rs7141529, rs2315654, rs12151618, rs747745, rs1009, rs2132276, rs2735839, rs11568818, rs684232, rs9364554, rs9830294, rs2660753, rs10807843, rs1933488, rs17467139, rs12947919, rs721048, rs385894, rs2331780, rs1894292, rs2107131, rs6545962, rs11649743, rs758643, rs2297434, rs902774, rs2647262, rs17224342, rs5918762, rs11672691, rs17138478, rs3019779, rs1873555, rs9457937, rs2838053, rs12946864, rs12475433, rs3765065, rs2018334, rs3771570, rs4871779, rs10875943, rs11601037, rs6489721, rs11168936, rs9297756, rs11900952, rs6569371, rs7752029, rs5934705, rs3745233, rs1482679, rs749264, rs6625760, rs5978944, rs2366711, rs5935063, rs10199796, rs2473057, rs4925094, and rs3096702, or a subset thereof.

Group 6

Further SNPs which are optionally included are selected from the group consisting of: rs12490248, rs4245739, rs10094059, rs306801, rs2823118, rs2025645, rs9359428, rs10178804, rs6090461, rs2270785, rs16901841, rs2465796, rs17256058, rs16849146, rs2269640, rs8044335, rs6530238, rs712242, rs9267911, rs11134144, rs12880777, rs7090755, rs132774, rs17779822, rs398146, rs4844228, rs4237185, rs7125415, rs1439024, rs6770955, rs11253002, rs4822763, rs2162185, rs12640320, rs5945637, rs3818714, rs6762443, rs10508678, rs2272668, rs2227270, rs6437715, rs3759129, rs1891158, rs7358335, rs12988652, rs3796547 rs7234917, rs6509345, rs966304, rs1515542, rs11631109, rs871688, s4382847, rs9972541, rs13113975, rs4119478, rs1380862, rs7529518, rs785437, rs1140809, rs4830488, rs10458360, rs2738571, rs11634741, rs1950198, rs539357, rs16887736, rs7658048, rs11222496, rs2207790, rs12506850, rs4512641, rs2813532, rs6934898, rs582598, rs10191478, rs10486562, rs17395631, rs7525167, rs12637074, rs10887926, rs7485441, rs1944047, rs7178085, rs17318620, rs10489871, rs2691274, rs6962297, rs1827611, rs4806120, rs7164364, rs2293710, rs13017302, rs4570588, rs2386841, rs40485, rs524908, rs10795841, rs4273907, rs12612891, rs10496470, rs6755901, rs1943821. rs13319878, rs6957416, rs12552397, rs6489794, rs4346531, rs7777631, rs1046011, rs16988279, rs986472, rs10508422, rs9456490, rs1295683, rs2449600, rs7075945, rs9358913, rs1477886, rs753032, rs409558, rs4246742, rs10060513, rs17070292, rs10826398, rs17744022, rs7801918, rs885479, rs1863610, rs3805284, rs10832514, rs2509867, rs2070874, rs2339654, rs12903579, rs11610799, rs2272316, rs6961773, rs2078277, rs17324573, rs6760417, rs2911756, rs12233245, rs896615, rs4760442, rs2087724, rs439378, rs4833103, rs6539333, rs4423250, rs12594014, rs17123359, rs12505546, and rs585197, or a subset thereof.

Group 7

Further SNPs which are optionally included are selected from the group consisting of: rs10060513, rs10086908, rs1009, rs10094059, rs10107982, rs1016343, rs10178804, rs10191478, rs10199796, rs1041449, rs10458360, rs1046011, rs10486562, rs10486567, rs10489871, rs10496470, rs10508422, rs10508678, rs1054564, rs10788160, rs10795841, rs10807843, rs10826398, rs10832514, rs10875943, rs10887926, rs10896437, rs10993994, rs11067228, rs11091768, rs11134144, rs11135910, rs11168936, rs11222496, rs11253002, rs1140809, rs11568818, rs11601037, rs11610799, rs11631109, rs11634741, rs11649743, rs11650494, rs11672691, rs11704416, rs11900952, rs12130132, rs12151618, rs12233245, rs1227732, rs12418451, rs12475433, rs12490248, rs12500426, rs12505546, rs12506850, rs12543663, rs12552397, rs12594014, rs12612891, rs12621278, rs12637074, rs12640320, rs1270884, rs12793759, rs12880777, rs12903579, rs12946864, rs12947919, rs1295683, rs12988652, rs13017302, rs13113975, rs13265330, rs132774, rs13319878, rs1354774, rs1363120, rs1380862, rs138213197, rs1439024, rs1477886, rs1482679, rs1515542, rs1558902, rs16849146, rs16860513, rs16887736, rs16896742, rs16901841, rs16901922, rs16901979, rs16902094, rs16988279, rs17021918, rs17070292, rs17123359, rs17138478, rs17224342, rs17256058, rs17318620, rs17324573, rs17395631, rs17467139, rs17744022, rs17779822, rs17832285, rs1827611, rs1863610, rs1873555, rs1891158, rs1894292, rs1933488, rs1943821, rs1944047, rs1950198, rs1992833, rs2018334, rs2025645, rs2028900, rs2047408, rs2070874, rs2078277, rs2087724, rs2107131, rs2132276, rs2162185, rs2207790, rs2227270, rs2269640, rs2270785, rs2272316, rs2272668, rs2273669, rs2293710, rs2297434, rs2315654, rs2331780, rs2339654, rs2366711, rs2386841, rs2405942, rs2449600, rs2465796, rs2473057, rs2509867, rs2647262, rs2659051, rs2660753, rs2691274, rs2710647, rs2735839, rs2736098, rs2738571, rs2813532, rs2823118, rs2838053, rs2911756, rs2928679, rs3019779, rs306801, rs3096702, rs3120137, rs3213764, rs3745233, rs3759129, rs3765065, rs3771570, rs3796547, rs3805284, rs3818714, rs3850699, rs385894, rs3863641, rs398146, rs401681, rs40485, rs409558, rs4119478, rs4237185, rs4245739, rs4246742, rs4273907, rs4346531, rs4382847, rs439378, rs4423250, rs4430796, rs4512641, rs4570588, rs4643253, rs4699312, rs4760442, rs4806120, rs4822763, rs4830488, rs4833103, rs4844228, rs4871779, rs4925094, rs524908, rs539357, rs582598, rs585197, rs5918762, rs5934705, rs5935063, rs5945619, rs5945637, rs5978944, rs6062509, rs6090461, rs620861, rs6437715, rs6465657, rs6489721, rs6489794, rs6509345, rs651164, rs6530238, rs6539333, rs6545962, rs6569371, rs6579002, rs6625760, rs6755901, rs6760417, rs6762443, rs6770955, rs6794467, rs684232, rs6869841, rs6934898, rs6957416, rs6961773, rs6962297, rs6983267, rs6983561, rs7075945, rs7090755, rs7102758, rs7106762, rs712242, rs7125415, rs7141529, rs7164364, rs7178085, rs721048, rs7213769, rs7234917, rs7241993, rs7358335, rs747745, rs7485441, rs749264, rs7525167, rs7529518, rs753032, rs758643, rs7611694, rs7658048, rs7679673, rs7752029, rs7777631, rs7801918, rs7818556, rs785437, rs8008270, rs8044335, rs871688, rs885479, rs888507, rs888663, rs896615, rs902774, rs9267911, rs9297746, rs9297756, rs9358913, rs9359428, rs9364554, rs9456490, rs9457937, rs9623117, rs966304, rs9830294, rs986472, rs9911515, and rs9972541, or a subset thereof.

Group 8

Further SNPs which are optionally included are selected from the group consisting of: rs4245739, rs13385191, rs1465618, rs6545977, rs721048, rs10187424, rs12621278, rs2292884, rs7584330, rs9311171, rs17181170, rs2660753, rs9284813, rs7629490, rs10934853, rs6763931, rs345013, rs10936632, rs17021918, rs12500426, rs7679673, rs2242652, rs12653946, rs2121875, rs4466137, rs37181, rs1983891, rs10498792, rs339331, rs651164, rs9364554, rs12155172, rs10486567, rs6465657, rs1512268, rs12543663, rs10086908, rs1016343, rs13252298, rs1456315, rs13254738, rs6983561, rs188140481, rs16902094, rs445114, rs6983267, rs7000448, rs1447295, rs4242382, rs4242384, rs7837688, rs817826, rs1571801, rs10993994, rs3123078, rs11199874, rs4962416, rs7127900, rs10896449, rs7931342, rs12418451, rs11228565, rs7130881, rs731236, rs10875943, rs902774, rs12827748, rs9600079, rs1529276, rs4775302, rs684232, rs7501939, rs4430796, rs11649743, rs138213197, rs11650494, rs7210100, rs1859962, rs103294, rs8102476, rs887391, rs2735839, rs9623117, rs742134, rs5759167, rs5945572, rs5945619, rs1327301, and rs5919432, or a subset thereof.

Group 9

Further SNPs which are optionally included are selected from the group consisting of: rs12621278, rs76862931, rs76264695, rs2293648, rs12053442, rs75166599, rs16860426, rs74461567, rs77167534, rs16860438, rs1574259, rs1574256, rs115112210, rs7584330, rs13390494, rs58643200, rs7606177, rs66509917, rs13432426, rs6757376, rs17021918, rs1139697, rs998071, rs10590, rs17021972, rs3762876, rs1043848, rs2136486, rs7679673, rs10007915, rs12653946, rs10866528, rs1983891, rs4714485, rs2104506, rs9369290, rs4714487, rs1886816, rs6458228, rs3747744, rs339331, rs339356, rs339353, rs1512658, rs2274911, rs6901971, rs339301, rs9364554, rs388170, rs3105751, rs7740824, rs10486567, rs7808935, rs67152137, rs6983267, rs12682374, rs7127900, rs11043135, rs11603101, rs7121039, rs7395734, rs7481129, rs10896449, rs7121816, rs7929962, rs11228565, rs7117034, rs8102476, rs8100395, rs7247241, rs7250689, rs34582151, rs11083450, rs12611084, and rs3786877, or a subset thereof.

### Sample Analyzers

It should be appreciated that any of the immunoassay methods disclosed herein may be performed or implemented using a device (e.g., a chip) and/or a sample analyzer. For example, a chip may be used to determine one or more characteristics of kallikrein proteins (markers) (e.g., levels of tPSA, fPSA, iPSA, free hK2, and/or total hK2) and may additionally be used to determine one or more characteristics of additional proteins (markers) (e.g, levels of MSMB and/or MIC-1). In some embodiments, the chip is not used to determine one or more characteristics of iPSA. In some embodiments, a system may include a sample analyzer, which for example, may be configured to analyze a sample on one or more chips (e.g., capable of analyzing antigen-antibody complexes, tracers, etc.). In some embodiments, an analyzer comprises an optical system including one or more light sources and/or one or more detectors configured for measuring levels of antigen-antibody complexes and/or tracers present on one or more chips. Furthermore, in some embodiments, systems are provided, which may include a processor or computer programmed to evaluate a predictive model (e.g., a logistic regression model) in electronic communication with the sample analyzer or other device for determining a probability of an event associated with prostate cancer based on levels of proteins (markers) (e.g., levels of tPSA, fPSA, iPSA, free hK2, and/or total hK2). In some embodiments, determining the probability of an event associated with prostate cancer is not based on levels of iPSA.

In one particular example, a system includes a sample analyzer comprising a housing and an opening in the housing configured to receive at least one solid support assay (e.g., one or more chips), wherein the housing includes a component configured to interface with a mating component on the at least one solid support assay (e.g., one or more chips) or an adaptor therefor to detect the presence of the at least one solid support assay within the housing. The system further includes an optical system positioned within the housing, the optical system including at least one light source and at least one detector spaced apart from the light source, wherein the light source is configured to pass light through the at least one solid support assay when it is inserted into the sample analyzer and wherein the detector is positioned opposite the light source to detect the amount of light that passes through the at least one solid support assay.

The methods and systems described herein may involve variety of different types of analyses, and can be used to determine a variety of different samples. In some cases, an analysis involves a chemical and/or biological reaction. In some embodiments, a chemical and/or biological reaction involves binding. Different types of binding may take place in or on the chips described herein.

Typical sample fluids include physiological fluids such as human or animal whole blood, blood serum, blood plasma, semen, tears, urine, sweat, saliva, cerebro-spinal fluid, vaginal secretions; in vitro fluids used in research or environmental fluids such as aqueous liquids suspected of being contaminated by the analyte.

In some embodiments, one or more reagents that can be used to determine the presence or concentration of an analyte in a sample (e.g., a binding partner of the analyte to be determined) is stored in or on the chip prior to first use in order to perform a specific test or assay. In cases where an antigen is being analyzed, a corresponding antibody or aptamer can be the binding partner associated with a surface of a solid support (e.g., the surface of a chip). If an antibody is the analyte, then an appropriate antigen or aptamer may be the binding partner associated with the surface. When a disease condition is being determined, it may be preferable to put the antigen on the surface and to test for an antibody that has been produced in the subject. It should be appreciated that while antibodies are referred to herein, antibody fragments may be used in combination with or in place of antibodies.

In some embodiments, a chip may be adapted and arranged to perform an analysis involving use of an opaque material on one or more regions of the chip. An opaque material may include a substance that interferes with the transmittance of light at one or more wavelengths. An opaque material does not merely refract light, but reduces the amount of transmission through the material by, for example, absorbing or reflecting light. Different opaque materials or different amounts of an opaque material may allow transmittance of less than, for example, 90, 80, 70, 60, 50, 40, 30, 20, 10 or 1 percent of the light illuminating the opaque material. Examples of opaque materials include molecular layers of metal (e.g., elemental metal), ceramic layers, polymeric layers, and layers of an opaque substance (e.g., a dye). The opaque material may, in some cases, be a metal that can be electrolessly deposited. These metals may include, for example, silver, copper, nickel, cobalt, palladium, and platinum.

The opaque material may include a series of discontinuous independent particles that together form an opaque layer, but in one embodiment, is a continuous material that takes on a generally planar shape. The opaque material may have a dimension (e.g., a width of length) of, for example, greater than or equal to 1 micron, greater than or equal to 5 microns, greater than 10 microns, greater than or equal to 25 microns, or greater than or equal to 50 microns. In some cases, the opaque material extends across the width of one or more regions (e.g., an analysis region or a liquid containment region) containing the opaque material. The opaque layer may have a thickness of, for example, less than or equal to 10 microns, less than or equal to 5 microns, less than or equal to 1 micron, less than or equal to 100 nanometers or less than or equal to 10 nanometers. Even at these small thicknesses, a detectable change in transmittance can be obtained. The opaque layer may provide an increase in assay sensitivity when compared to techniques that do not form an opaque layer.

A variety of determination (e.g., measuring, quantifying, detecting, and qualifying) techniques may be used, e.g., to analyze a sample component or other component or condition associated with a chip described herein. Determination techniques may include optically-based techniques such as light transmission, light absorbance, light scattering, light reflection and visual techniques. Determination techniques may also include luminescence techniques such as photoluminescence (e.g., fluorescence), chemiluminescence, bioluminescence, and/or electrochemiluminescence. In other embodiments, determination techniques may measure conductivity or resistance. As such, an analyzer may be configured to include such and other suitable detection systems.

Different optical detection techniques provide a number of options for determining reaction (e.g., assay) results. In some embodiments, the measurement of transmission or absorbance means that light can be detected at the same wavelength at which it is emitted from a light source. Although the light source can be a narrow band source emitting at a single wavelength it may also may be a broad spectrum source, emitting over a range of wavelengths. In some embodiments, a system may be operated with a minimum of optical devices (e.g., a simplified optical detector). For instance, the determining device may be free of a photomultiplier, may be free of a wavelength selector such as a grating, prism or filter, may be free of a device to direct or columnate light such as a columnator, or may be free of magnifying optics (e.g., lenses). Elimination or reduction of these features can result in a less expensive device.

An optical system may be positioned in the housing of an analyzer. The optical system may include, for example, at least a first light source and a detector spaced at the same position, near to, or apart from the first light source. The first light source may be configured to pass light through one or more liquid containment regions of the chip when the chip is inserted into the analyzer. The first detector may be positioned opposite the first light source to detect the amount of light that passes through the first analysis region of the chip when it is inserted. In another embodiment, the light source and the detector may be on the same side of the chip. For example, the light source and the detector may be positioned above the chip. As another example, the light source and the detector may both be positioned below the chip. It should be appreciated that in other embodiments, the number of light sources and detectors may vary as the invention is not so limited. Each chip may include a plurality of liquid containment regions (e.g., two or more liquid containment regions) and the one or more chips or a chip adapter may be positioned within the analyzer such that each liquid containment region aligns with at least one light source and corresponding detector. In some embodiments, the light source includes an optical aperture which may help direct light from the light source to a particular region within a liquid containment region of the chip.

In one embodiment, the light source is one or more lasers, e.g., a gas laser or a solid state laser. For example, the laser could be a helium-neon (HeNe) laser, a carbon dioxide (CO₂) laser, a carbon monoxide (CO) laser, a nitrogen laser, or a transversely excited atmospheric (TEA) laser. The power output of the laser may be, for example, between 0.1 mW and 100 mW. For example, the power output of the laser may be 0.1-0.5 mW, 0.5-25 mW, 25-50 mW, 50-75 mW, or 75-100 mW. As a specific example, the power output of the laser may be 0.1 mW, 0.2 mW, 0.3 mW, 0.4 mW, 0.5 mW, 1 mW, 2 mW, 3 mW, 4 mW, 5 mW, 6 mW, 7 mW, 8 mW, 9 mW, 10 mW, 20 mW, 30 mW, 40 mW, 50 mW, 60 mW, 70 mW, 80 mW, 90 mW, or 100 mW.

In one embodiment, the light sources are light emitting diodes (LEDs) or laser diodes. For example, an InGaAlP red semiconductor laser diode emitting at 654 nm may be used. Other light sources can also be used. The light source may be positioned within a nest or housing. The nest or housing may include a narrow aperture or thin tube that may assist in collimating light. The light sources may be positioned above where the one or more chips are inserted into the analyzer such that the light source shines down onto the top surface of the one or more chips. The light sources may be positioned below where the one or more chips are inserted into the analyzer such that the light source shines up onto the bottom surface of the one or more chips. Other suitable configurations of the light source with respect to the one or more chips are also possible.

It should be appreciated that the wavelength of the light sources may vary as the invention is not so limited. For example, in one embodiment, the wavelength of the light source is approximately 670 nm, and in another embodiment, the wavelength of the light source is approximately 650 nm. In another embodiment, the wavelength of the light source is approximately 488 nm, 532 nm, 594 nm, or 633 nm. It should be appreciated that in one embodiment, the wavelength of each light source may be different such that each analysis region of the one or more chips receives a different light wavelength. In other embodiments, however, the wavelength of each light source may be the same such that each analysis region of the one or more chips receives the same light wavelength. Combinations of the same and different wavelengths of light sources are also possible. One or more filters may be used with the laser. As a non-limiting example, the filter may be a CY5 (692/40), ROX (635/35), CY3 (575/50), or FITC (535/25) filter.

The analyzer may have a working distance of any value. As a non-limiting example, the working distance may be a value of 0.5-10 mm. For example, the depth of focus may be about 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

The depth of focus of the analyzer may be any value. As a non-limiting example, the depth of focus (80-100% intensity) may be about 10-1000 μm. For example, the depth of focus (80-100% intensity) may be about 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm.

As mentioned, a detector may be spaced near to or apart from a light source to detect the amount of light that is emitted from an analysis region. In one embodiment, one or more of the detectors are photodetectors (e.g., photomultiplier tubes or photodiodes). In certain embodiments, the photodetector may be any suitable device capable of detecting the emission of light from the one or more analysis regions. In certain embodiments, the photodetector may be any suitable device capable of detecting the transmission of light from the one or more analysis regions. One type of photodetector is an optical integrated circuit (IC) including a photodiode having a peak sensitivity at 700 nm, an amplifier and a voltage regulator. The detector may be positioned within a nest or housing which may include a narrow aperture or thin tube to ensure that only light from analysis region 709 is measured at the detector 884. If the light source is pulse modulated, the photodetector may include a filter to remove the effect of light that is not at the selected frequency. When multiple and neighboring signals are detected at the same time, the light source used for each analysis region (e.g., detection region) can be modulated at a frequency sufficiently different from that of its neighboring light source. In this configuration, the each detector can be configured (e.g., using software) to select for its attributed light source, thereby avoiding interfering light form neighboring optical pairs. There may be one or more than one detectors (i.e., one or more photomultiplier tubes) such as two, three, four, or five detectors.

The amount of light transmitted through or emitted from an analysis region of the chip may be used to determine information about not only the sample, but also information about specific processes related to the one or more chips. In certain embodiments, quality control or abnormalities in chip can be determined. For example, feedback from a control analysis region can be used to determine whether abnormalities have occurred in the manufacturing or storage of the chip.

In one embodiment, the analyzer includes a temperature regulating system positioned within the housing which may be configured to regulate the temperature within the analyzer. For certain sample analysis, the sample may need to be kept within a certain temperature range. For example, in one embodiment, it is desirable to maintain the temperature within the analyzer at approximately 37° C. Accordingly, in one embodiment, the temperature regulating system includes a heater configured to heat the one or more chips. In one embodiment, the heater is a resistive heater which may be positioned on the underside of where the one or more chips are placed in the analyzer. In one embodiment, the temperature regulating system also includes a thermistor to measure the temperature of one or more of the chips and a controller circuit may be provided to control the temperature.

In one embodiment, the passive flow of air within the analyzer may act to cool the air within the analyzer if needed. A fan may optionally be provided in the analyzer to lower the temperature within the analyzer. In some embodiments, the temperature regulating system may include Peltier thermoelectric heaters and/or coolers within the analyzer.

The information from or associated with an identifier can, in some embodiments, be stored, for example in computer memory or on a computer readable medium, for future reference and record-keeping purposes. For example, certain control systems may employ information from or associated with identifiers to identify which components (e.g., chips) or types of chips were used in a particular analysis, the date, time, and duration of use, the conditions of use, etc. Such information may be used, for example, to determine whether one or more components of the analyzer should be cleaned or replaced. Optionally, a control system or any other suitable system could generate a report from gathered information, including information encoded by or associated with the identifiers, that may be used in providing proof of compliance with regulatory standards or verification of quality control.

Information encoded on or associated with an identifier may also be used, for example, to determine whether the component associated with the identifier (e.g., a chip) is authentic or counterfeit. In some embodiments, the determination of the presence of a counterfeit component causes system lockout. In one example, the identifier may contain a unique identity code. In this example, the process control software or analyzer would not permit system startup (e.g., the system may be disabled) if a foreign or mismatched identity code (or no identity code) was detected.

In certain embodiments, the information obtained from or associated with an identifier can be used to verify the identity of a customer to whom the chip and/or analyzer is sold or for whom a biological, chemical, or pharmaceutical process is to be performed. In some cases, the information obtained from or associated with an identifier is used as part of a process of gathering data for troubleshooting a system. The identifier may also contain or be associated with information such as batch histories, assembly process and instrumentation diagrams (P and IDs), troubleshooting histories, among others. Troubleshooting a system may be accomplished, in some cases, via remote access or include the use of diagnostic software.

In one embodiment, the analyzer includes a user interface, which may be positioned within the housing and configured for a user to input information into the sample analyzer. In one embodiment, the user interface is a touch screen.

The touch screen may guide a user through the operation of the analyzer, providing text and/or graphical instructions for use of the analyzer. The touch screen user interface may, for example, guide the user to insert the one or more chips or chip adapter into the analyzer. It may then guide the user to input the subject's name or other subject identification source/number into the analyzer (e.g., age, results of a DRE exam, etc.). It should be appreciated that the subject information such as name, date of birth, and/or subject ID number may be inputted into the touch screen user interface to identify the subject. The touch screen may indicate the amount of time remaining to complete the analysis of the sample. The touch screen user interface may then illustrates the results of the sample analysis along with the subject's name or other identifying information.

In another embodiment, the user interface may be configured differently, such as with an LCD display and a single button scroll through menu. In another embodiment, the user interface may simply include a start button to activate the analyzer. In other embodiments, the user interface from separate independent devices (such as a smart phone or mobile computer) can be used to interface with the analyzer.

The above-described analyzer may be used in a variety of ways to process and analyze a sample placed within the analyzer. In one particular embodiment, once a mechanical component configured to interface with the one or more chips or the chip adapter indicates that the one or more chips or chip adapter is properly loaded in the analyzer, the identification reader reads and identifies information associated with the one or more chips. The analyzer may be configured to compare the information to data stored in a control system to ensure that it has calibration information for this particular sample. In the event that the analyzer does not have the proper calibration information, the analyzer may output a request to the user to upload the specific information needed. The analyzer may also be configured to review expiration date information associated with the one or more chips and cancel the analysis if the expiration date has passed.

In one embodiment, the optical system may take initial measurements to obtain reference readings. Such reference readings may be taken both with the light sources activated and deactivated.

In one particular set of embodiments, the analyzer is used to measure the level of iPSA, fPSA, tPSA, total hK2 and/or free hK2 in a blood sample. In one particular set of embodiments, the analyzer is used to measure the level of fPSA, tPSA, total hK2 and/or free hK2 in a blood sample. In certain embodiments, the analyzer is not used to measure the level of iPSA. In some embodiments, three, four, five, six or more analyses may be utilized to analyze the sample.

The system may include a user interface associated with the housing for inputting at least one clinical factor (e.g., the age of a person). The system may include a processor in electronic communication with the analyzer, the processor programmed to evaluate a logistic regression model as described herein in combination with information indicative of levels of one or more kallikrein proteins (markers) selected from: tPSA, fPSA, iPSA, total hK2, and/or free hK2 in a blood plasma sample of a subject previously diagnosed as having a non-aggressive prostate cancer. In some embodiments, the system may include a processor in electronic communication with the analyzer, the processor programmed to evaluate a logistic regression model as described herein in combination with information indicative of levels of one or more kallikrein proteins (markers) selected from: tPSA, fPSA, total hK2, and/or free hK2 in a blood plasma sample of a subject previously diagnosed as having a non-aggressive prostate cancer. In certain embodiments, the information indicative of levels of one or more kallikrein proteins (markers) is not information about iPSA.

Non-limiting examples of suitable devices are disclosed in US Patent Application Publication Number US 2013/0273643, entitled “Methods and Apparatuses for Predicting Risk of Prostate Cancer and Prostate Gland Volume,” which published on Oct. 17, 2013, and U.S. Pat. No. 8,765,062, entitled “Systems and Devices for Analysis of Samples”, which issued on Jul. 1, 2014, the contents of which are incorporated herein by reference in their entirety for all purposes. It should be appreciated, however, that other types of device may also be used (e.g., any and all examples of slide readers, plate readers, analyzers for microwell ELISA-type assays, etc.) as the disclosure is not limited in this respect.

An analyzer may be used with any of the embodiments described herein. The analyzer may include a housing which is configured to cover or retain the components of the analyzer which are discussed in greater detail below. An opening in the housing may be configured to receive one or more chips or a chip adapter. As set forth in greater detail below, the analyzer may also include a user interface positioned within the housing which is configured for a user to input information into the sample analyzer. In this particular embodiment, the user interface includes a touch screen, but as discussed below, the user interface may be configured differently.

In some embodiments, the analyzer may include an identification reader configured to read information associated with the one or more chips and/or a chip adapter and/or a mechanical subsystem which includes a component configured to interface with the one or more chips or a chip adapter to detect the one or more chips or a chip adapter within the housing. As mentioned above, an opening in the housing is configured to receive one or more chips or a chip adapter. The opening may be configured as an elongated slot. The opening may be configured in this manner to receive a substantially card-shaped or slide-shaped chip. The opening may be configured in this manner to receive a substantially card-shaped chip or slide adapter. It should be appreciated that in other embodiments, the opening may be shaped and configured differently as the invention is not so limited.

As mentioned above, the analyzer may be configured to receive a variety of types of chips or chip adapters (e.g., microarray devices). The chip may be substantially card-shaped (i.e., similar to a card key) or slide shaped (e.g., similar to a glass slide used for microscopy) having a substantially rigid plate-like structure.

As used herein, “prior to first use of the chip” means a time or times before the chip is first used by an intended user after commercial sale. First use may include any step(s) requiring manipulation of the device by a user. For example, first use may involve one or more steps such as opening the packaging containing one or more chips described herein, preparation of the chip (e.g., loading of reagents into or onto the chip) before analysis of a sample, loading of a sample onto the chip, preparation of a sample in a region of the chip, performing a reaction with a sample, detection of a sample, etc. First use, in this context, does not include manufacture or other preparatory or quality control steps taken by the manufacturer of the chip. Those of ordinary skill in the art are well aware of the meaning of first use in this context, and will be able easily to determine whether a chip of the invention has or has not experienced first use. In one set of embodiments, chips of the invention are disposable after first use (e.g., after completion of an assay), and it is particularly evident when such devices are first used, because it is typically impractical to use the devices at all (e.g., for performing a second assay) after first use.

The assay system described herein may include, for example, a chip or chip adapter operatively associated with one or more components such as one or more light sources, detection systems (e.g., for detecting one or more fluids and/or processes), and/or a temperature regulating systems or fans (e.g., to heat and/or cool one or more regions of the one or more chips). The components may be external or internal to the device, and may optionally include one or more processors for controlling the component or system of components. In certain embodiments, one or more such components and/or processors are associated with a sample analyzer configured to process and/or analyze a sample contained in or on one or more chips. The processor may optionally be programmed to evaluate a linear regression model as described herein.

In general, as used herein, a component that is “operatively associated with” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected (e.g., via channels such as tubing) so as to cause or enable the components so associated to perform their intended functionality.

The components of the analyzer, as well as other optional components such as those described herein, may be operatively associated with a control system. In some embodiments, the control system may be used to control fluids and/or conduct quality control by the use of feedback from one or more events taking place in the system. For instance, the control system may be configured to receive input signals from the one or more components, to calculate and/or control various parameters, to compare one or more signals or a pattern of signals with signals preprogrammed into the control system, and/or to send signals to one or more components to control operation of the system. The control system may also be optionally associated with other components such as a user interface, an identification system, an external communication unit (e.g., a USB), and/or other components, as described in more detail below.

One or more chips or a chip adapter (e.g., one or more microarrays) may have any suitable configuration of channels and/or components for performing a desired analysis. In one set of embodiments, one or more chips or a chip adapter contains stored reagents that can be used for performing a chemical and/or biological reaction (e.g., an immunoassay), e.g., as described in more detail herein.

### Predictive Models

Aspects of the disclosure provide computer implemented methods for determining a probability of an event associated with prostate cancer, such as an upgrade from non-aggressive to aggressive prostate cancer. Such methods may involve receiving, via an input interface, information indicative of the level of tPSA present in a blood plasma sample of a subject and receiving, via an input interface, information about whether the subject had a prior biopsy of prostate tissue. In some embodiments, the methods further involve evaluating, using at least one processor, a suitable predictive model (e.g., a logistic regression model) based, at least in part, on the received information to determine a probability of the event associated with prostate cancer in the subject. The predictive model may generate the probability of the event associated with prostate cancer based, at least in part, on measured levels of tPSA and information about whether the subject had a prior biopsy of prostate tissue. The predictive model may generate the probability of the event associated with prostate cancer based, at least in part, on measured levels of tPSA, fPSA, iPSA, free hK2, total hK2, MSMB, and/or MIC-1 and information about whether the subject had a prior biopsy of prostate tissue. The predictive model may generate the probability of the event associated with prostate cancer based, at least in part, on measured levels of tPSA, fPSA, free hK2, total hK2, MSMB, and/or MIC-1 and information about whether the subject had a prior biopsy of prostate tissue. In some embodiments, the predictive model is not based (even in part) on measured levels of iPSA.

One or more values representing subject data corresponding to age, digital examination status and/or prior biopsy status may be received by at least one processor for processing using one or more of the techniques described herein. First, one or more values representing protein (marker) data for tPSA, fPSA, iPSA, free hK2, total hK2, MSMB, and/or MIC-1 are received by the at least one processor. Alternatively, one or more values representing protein (marker) data for tPSA, fPSA, free hK2, total hK2, MSMB, and/or MIC-1 are received by the at least one processor. In some embodiments, the values do not represent data for iPSA. The values may be received in any suitable way including, but not limited to, through a local input interface such as a keyboard, touch screen, microphone, or other input device, from a network-connected interface that receives the value(s) from a device located remote from the processor(s), or directly from one or more detectors that measure the blood protein (marker) value(s) (e.g., in an implementation where the processor(s) are integrated with a measurement device that includes the one or more detectors).

After receiving the value(s) for tPSA, the process proceeds such that if levels of tPSA are above a threshold (e.g., 25 ng/mL), then a first predictive model may be selected and, if levels of tPSA are at or below the threshold, then a second predictive model may be selected. Accordingly, if the levels of tPSA are above the threshold level then a predictive model may be selected that is based DRE status, prior biopsy status and tPSA levels. Alternatively, if the levels of tPSA are at or below the threshold level, then a predictive model is selected based on DRE status, prior biopsy status and tPSA, fPSA, iPSA free hK2, total hK2, MSMB, and/or MIC-1 levels. In some embodiments, if the levels of tPSA are at or below the threshold level, then a predictive model is selected based on DRE status, prior biopsy status and tPSA, fPSA, free hK2, total hK2, MSMB, and/or MIC-1 levels. In certain embodiments, the predictive model is not based on iPSA levels. A predictive model is then used to determine the probability that a subject has a prostate cancer. The prediction may be for a cancer of any grade or for a cancer of high grade or for an upgrade from non-aggressive to aggressive prostate cancer depending on the model used.

After determining a probability of an event associated with prostate cancer (e.g., an upgrade from non-aggressive prostate cancer to aggressive prostate cancer), output to a user (e.g., a physician, a subject) may be provided to guide a further diagnostic procedure and/or treatment decisions. The probability may be output in any suitable way. For example, in some embodiments, the probability may be output by displaying a numeric value representing the probability on a display screen of a device. In other embodiments, the probability may be output using one or more lights or other visual indicators on a device. In yet other embodiments, the probability may be provided using audio output, tactile output, or some combination of one or more of audio, tactile, and visual output. In some embodiments, outputting the probability comprises sending information to a network-connected device to inform a user about the determined probability. For example, the probability may be determined by one or more processors located at a remote site, and an indication of the probability may be sent to an electronic device of a user (e.g., a physician) using one or more networks, in response to determining the probability at the remote site. The electronic device that provides output to a user in accordance with the techniques described herein may be any suitable device including, but not limited to, a laptop, desktop, or tablet computer, a smartphone, a pager, a personal digital assistant, and an electronic display.

In some embodiments, the probability of the event associated with prostate cancer is determined in accordance with equation (I), reproduced below:

$$Probability = \frac{{e}^{L}}{1 + {e}^{L}} \qquad ( 1 )$$

where the log it (L) is determined using any of a plurality of logistic regression models.

The data may be combined using any type of algorithmic analysis of any set or subset of results. This algorithmic analysis may be a linear combination of the data wherein the linear combination improves the diagnostic performance (e.g., as measured using ROC-AUC). Other possible methods for analyzing the data and/or combining the data into a model capable of producing a diagnostic estimate may include (but are not limited to) non-linear polynomials, support vector machines, neural network classifiers, discriminant analysis, random forest, gradient boosting, partial least squares, ridge regression, lasso, elastic nets, and/or k-nearest neighbors. Specific methods for predicting or classifying a particular outcome may also be found, for example, in “The Elements of Statistical Learning: Data Mining, Inference, and Prediction, Second Edition” by T Hastie et al., Springer Series in Statistics, ISBN 978-0387848570, which is incorporated by reference herein in its entirety.

As a non-limiting example, a linear model could be designed using only information regarding the age, family history, and the ratio of free PSA to total PSA (free/total PSA) for a subject. The first linear model would be defined as: LM1=1.07679−0.00118523*[AGE]+0.0952954*[FAMILYHISTORY]−0.0234183*[free/total PSA].

A second linear model could be designed using age and family history in addition to the levels of tPSA, fPSA, hK2 (free hK2 or total hK2), and the ratio of free PSA to total PSA (free/total PSA) for a subject. The second linear model would be defined as: LM2=0.806743−0.000112063*[AGE]+0.0541963*[FAMILYHISTORY]+0.000537*[tPSA]+0.0605211*[fPSA]−0.0218285*[free/total PSA]+0.624642*[hK2].

A third linear model could be designed using previous biopsy information (“PrevBiop” where the subject has previously had a prostate biopsy before, indicated by a 1, or has not previously had a biopsy, indicated by a 0), and ‘GenScore’ is the genetic score variable computed as described in the public report “Polygenic Risk Score Improves Prostate Cancer Risk Prediction: Results from the Stockholm-I Cohort Study” by Markus Aly et al., European Urology; 60 (2011) 21-28, which takes results of SNP testing into account. The parameters “[hK2]”, “[fPSA]”, “[iPSA]”, “[MIC-1]”, “[MSMB]”, and “[tPSA]” refer to the respective measured values of these biomarkers, “free/total PSA” is the ratio of free PSA to total PSA, and “age” is the age of the subject. Further, “[hK2]” may refer to the measured value of free hK2 or total hK2. The third linear model would be defined as: LM3=0.0275109+0.4272770*PrevBiop+0.0006496*[tPSA]++0.0868130*GenScore−0.0334401*[hK2]+0.0082864*[iPSA]+0.0110069*[MIC-1]+0.0069329*[MSMB]+0.0084636*age−0.0018337*[fPSA]−1.6079442*(free/total PSA). In certain cases, the linear model does not include an iPSA component.

A fourth linear model could be designed using results from one or more parameters of markers that are kallikrein proteins. The parameters “[hK2]”, “[fPSA]”, “[iPSA]”, “[MIC-1]”, “[MSMB]”, and “[tPSA]” refer to the respective measured values of these biomarkers, “free/total PSA” is the ratio of free PSA to total PSA, and “age” is the age of the subject. Further, “[hK2]” may refer to the measured value of free hK2 or total hK2. In certain cases, the linear model does not include an iPSA component. If values for one or more of the kallikrein proteins (markers) are missing or suspected to be faulty or compromised, the kallikrein score (here shown as K, which represents the kallikrein score using all discussed proteins (markers) and K₁-K₃, which represents the kallikrein score using a subset of the markers) would vary according to the following formulae:

K=(0.07316*[tPSA]−0.13778*[fPSA]+0.01293*[hK2]+0.08323[iPSA]−0.01844*[free/total PSA])/0.07316−0.13778+0.01293+0.08323−0.01844)

K₁=(0.07316*[tPSA]−0.13778*[fPSA]+0.08323*[iPSA]−0.01844*[free/total PSA])/0.07316−0.13778+0.08323−0.01844)

K₂=(0.07316*[PSA]−0.13778*[fPSA]−0.01844*[free/total PSA])/(0.07316−0.13778−0.01844)

K₃=(0.07316*[tPSA]−0.13778*[fPSA])/(0.07316−0.13778)

The predictive model for assessing risk of prostate cancer would then be: LM4=(kallikrein score)*C1+GenScore*C2+[MIC-1]*C3+[MSMB]*C4+age*C5+C6 wherein C1-C6 are each constants for adjusting the contribution of each component.

The algorithm which integrates data from the different categories into a single value being indicative of if the subject is likely to suffer from solid tumor cancer may be, for example, a nonlinear function in which the dependency of different categories is employed for further increasing the diagnostic performance of the method. The algorithm used for predicting the risk for prostate cancer may also be improved through the use of transformed variables (e.g., through the use of log₁₀ (PSA) values). Transformation may be beneficial for variables with abnormal distribution. Possible variable transformations include, but are not limited to, logarithm, inverse, square, and square root transformations. In certain embodiments, the algorithm does not include an iPSA component.

Non-limiting examples of different types of logistic regression models that may be used in accordance with the techniques described herein include:

1. Simple Model (tPSA Only)

L=β₀+β₁(Age)+β₂(tPSA)+β₃(priorbx) (2)

or

L=β₀+β₁tpsa+β₂dre_{neg}+β₃dreₚₒₛ+β₁priorbx (3)

### 2. Four Assay Model Using Free/Total Ratio

In this model, the ratio of free PSA to total PSA is substituted for the free PSA term.

$$L = {β}_{0} + {β}_{1} ( Age ) + {β}_{2} ( tPSA ) + {β}_{3} ( \frac{fPSA}{tPSA} ) + {β}_{4} ( iPSA ) + {β}_{5} ( hK 2 ) + {β}_{6} ( priorbx ) \qquad ( 4 )$$

### 3. Four Assay Model Using Log(tPSA) and Free/Total Ratio

In this model, the log of tPSA is substituted for the tPSA term to account for the increased contribution of this predictive factor.

$$L = {β}_{0} + {β}_{1} ( Age ) + {β}_{2} ( log [ tPSA ] ) + {β}_{3} ( \frac{fPSA}{tPSA} ) + {β}_{4} ( iPSA ) + {β}_{5} ( hK 2 ) + {β}_{6} ( priorbx ) \qquad ( 5 )$$

### 4. Polynomial Model

In this model, additional non-linear terms for tPSA and fPSA are included. In the example equation provided below, the square of tPSA is used to emphasize the direct relationship between this term and risk of prostate cancer, and the square root of the free/total PSA term is used to reflect the inverse association of this term with risk. It should be appreciated however, that polynomial terms of higher order (e.g., cubic) may also be included in some embodiments.

$$L = {β}_{0} + {β}_{1} ( Age ) + {β}_{2} ( tPSA ) + {β}_{3} ( fPSA ) + {β}_{4} ( iPSA ) + {β}_{5} ( hK 2 ) + {β}_{6} ( {tPSA}^{2} ) + {β}_{7} ( \sqrt{\frac{fPSA}{tPSA}} ) + {β}_{8} ( priorbx ) \qquad ( 6 )$$

### 5. Linear Splines for all Four Assays

In this model, linear splines are added, with a single knot at the median value. The splines may be determined using the following equations:

sp1(x)=x if x<knot

sp1(x)=knot if x≥knot

sp2(x)=0 if x<knot

sp2(x)=x−knot if x≥knot (7)

with the model being represented as:

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA])+β₁₀(sp1[iPSA])+β₁₁(sp2[iPSA])++β₁₂(sp1[hK2])+β₁₃(sp2[hK2])+β₁₄(priorbx) (8)

6. Linear Splines for tPSA and fPSA

In this model, linear splines are included only for tPSA and fPSA to reduce the number of variables and simplify the model.

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA])+β₁₀(priorbx) (9)

In the equations above “priorbx” is a binary value indicate of whether a subject had a prior biopsy to detect prostate cancer. A value of 1 indicates that a prior biopsy occurred and a value of 0 indicates that the prior biopsy did not occur.

### 7. Cubic Splines for all Four Assays

In this model, cubic splines are included for each term. In the example provided below, a cubic spline with four knots is described. It should be appreciated, however, that a cubic spline using any suitable number of knots including, but not limited to, five knots, six knots, seven knots, and eight knots, may alternatively be used. The splines may be determined using the following

$$\begin{matrix}sp [ x ] 1 = {max ( [ x ] - knot 1 , 0 )}^{3} - {max ( [ x ] - knot 3 , 0 )}^{3} \frac{knot 4 - knot 1}{knot 4 - knot 3} + {max ( [ x ] - knot 4 , 0 )}^{3} \frac{knot 3 - knot 1}{knot 4 - knot 3} & equations ( 10 ) \\ sp [ x ] 2 = {max ( [ x ] - knot 2 , 0 )}^{3} - {max ( [ x ] - knot 3 , 0 )}^{3} \frac{knot 4 - knot 2}{knot 4 - knot 3} + {max ( [ x ] - knot 2 , 0 )}^{3} \frac{knot 3 - knot 2}{knot 4 - knot 3} & ( 11 )\end{matrix}$$

where knot1 and knot4 are external knots for the cubic spline, and knot2 and knot3 are internal knots for the cubic spline. The external knots may be set as the minimum and maximum levels of tPSA, fPSA, iPSA, or hK2 (free or total hK2) in a population. An internal knot (e.g., knot2) may be set as the 33.3 percentile value of tPSA, fPSA, iPSA, or hK2 (free or total hK2) levels in a population. Another internal knot (e.g., knot3) may be set as the 66.6 percentile value of tPSA, fPSA, iPSA, or hK2 (free or total hK2) levels in a population.

In some embodiments, the internal knots are specified within the range of between about 2 to about 8 and between about 3 to about 6 for tPSA, between about 0.25 to about 2 and between about 0.5 to about 1.5 for fPSA, between about 0.2 to about 0.5 and between about 0.4 to about 0.8 for iPSA, and between about 0.02 to about 0.04 and between about 0.04 to about 0.08 for hK2 (free or total hK2). For example, in one implementation, values of 3.92 and 5.61 are used for the internal knots for tPSA, values of 0.82 and 1.21 are used for the internal knots for fPSA, values of 0.3 and 0.51 are used for the internal knots of iPSA, and values of 0.036 and 0.056 are used for the internal knots of hK2 (free or total hK2).

In certain embodiments, one or more internal knots for tPSA may independently be in the range of between about 3 to about 5, between about 3 to about 6, between about 2.5 to about 6, between about 2.5 to about 6.5, between about 5 to about 8, between about 5.5 to about 8, between about 5 to about 9, between about 5 to about 10, between about 1 to about 5, between about 1 to about 4, and between about 1 to about 3. Other ranges are also possible.

In certain embodiments, one or more internal knots for fPSA may independently be in the range of between about 0.1 to about 1.0, between about 0.1 to about 1.2, between about 0.3 to about 0.8, between about 0.4 to about 0.9, between about 0.5 to about 1.2, between about 0.7 to about 1.4, between about 0.7 to about 0.9, between about 1.1 to about 1.6, between about 1.1 to about 1.2, and between about 1.1 to about 2. Other ranges are also possible.

In certain embodiments, one or more internal knots for iPSA may independently be in the range of between about 0.05 to about 0.5, between about 0.1 to about 0.5, between about 0.2 to about 0.5, between about 0.1 to about 0.8, between about 0.2 to about 0.8, between about 0.4 to about 0.8, between about 0.4 to about 1.0, between about 0.3 to about 0.6, between about 0.5 to about 1.0, and between about 0.6 to about 0.8. Other ranges are also possible. In certain cases, the model does not include an iPSA component or internal knot.

In certain embodiments, one or more internal knots for hK2 (free or total hK2) may independently be in the range of between about 0.01 to about 0.03, between about 0.01 to about 0.04, between about 0.01 to about 0.05, between about 0.02 to about 0.05, between about 0.02 to about 0.06, between about 0.03 to about 0.05, between about 0.4 to about 0.07, between about 0.04 to about 1.0, between about 0.5 to about 1.0, and between about 0.6 to about 1.0. Other ranges are also possible.

As discussed above, cubic splines incorporating any suitable number of internal knots (e.g., three, four, five, six internal knots) may be used, and the example of a cubic spline including two internal knots is provided merely for illustration and not limitation. In embodiments that include more than two internal knots, the knots may be placed within one or more of the ranges discussed above, or in some other suitable range. For example, in some embodiments, the knots may be specified such that the length of the segments of the spline between each of the pairs of neighboring knots is essentially equal.

The model may be represented as:

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA])+β₁₀(sp1[iPSA])+β₁₁(sp2[iPSA])+β₁₂(sp1[hK2])+β₁₃(sp2[hK2])+β₁₄(priorbx) (12)

8. tPSA Threshold Model

In some embodiments, the model selected may depend on the whether or not a threshold level of tPSA is detected in sample. In some embodiments, if the level of tPSA is above a threshold in a sample, then the predictive model is as follows:

L=β₀+β₁(tPSA)+β₂(DRE)_{neg}+β₃(DRE)ₚₒₛ+β₄(priorbx) (13)

In some embodiments, the range of values of the weighting coefficients in this model are as set forth in Table 1 below. Coefficients suitable for determining the probability that a prostate tissue biopsy will have a cancer of any grade are shown in the second and third columns; whereas coefficients suitable for determining the probability that a prostate tissue biopsy will have a cancer of high grade are shown in the fourth and fifth columns.

TABLE 1
Weighting Coefficients to be used when
level of tPSA is greater than Threshold

| Weighting | | | Cancer of High Grade | |
|---|---|---|---|---|
| Coefficient | Cancer of Any Grade | | (Gleason Score >= 7.0) | |
| Ranges | Low | High | Low | High |
| β₀ | −1.22E+00 | −9.07E−01 | 7.83E−01 | 9.31E−01 |
| β₁ | 1.04E−01 | 1.22E−01 | 1.24E−02 | 1.59E−02 |
| β₂ | −6.62E−02 | −4.99E−02 | −2.19E−01 | −1.72E−01 |
| β₃ | 1.34E−01 | 1.71E−01 | 5.23E−01 | 6.44E−01 |
| β₄ | −1.30E+00 | −8.91E−01 | −1.94E+00 | −1.68E+00 |

In some embodiments, if the level of tPSA detected in a sample is less than or equal to a threshold level, then the predictive model is as follows:

L=β₀+β₁(Age)+β₂(tPSA)+β₃sp1(tPSA)+β₄sp2(tPSA)+β₅(fPSA)+β₆(fPSA)+β₇sp2(fPSA)+β₈(tPSA)+β₉(hK2)+β₁₀(DRE_{neg})+β₁₁(DREₚₒₛ)+β₁₂(priorbx) (14)

In some embodiments, the range of values of the weighting coefficients in this model are as set forth in Table 2 below. Coefficients suitable for determining the probability that a prostate tissue biopsy will have a cancer of any grade are shown in the second and third columns; whereas coefficients suitable for determining the probability that a prostate tissue biopsy will have a cancer of high grade are shown in the fourth and fifth columns.

TABLE 2
Weighting Coefficients to be used when level
of tPSA is less than or equal to a threshold

| Weighting | | | Cancer of High Grade | |
|---|---|---|---|---|
| Coefficient | Cancer of Any Grade | | (Gleason Score > 7.0) | |
| Ranges | Low | High | Low | High |
| β₀ | −2.86E+00 | −1.97E+00 | −7.35E+00 | −6.00E+00 |
| β₁ | 2.88E−01 | 4.03E−01 | 4.79E−02 | 6.38E−02 |
| β₂ | 3.76E−01 | 4.72E−01 | 7.44E−01 | 9.19E−01 |
| β₃ | −2.18E−04 | −1.78E−04 | −6.43E−03 | −4.32E−03 |
| β₄ | −1.22E−03 | −9.46E−04 | 1.20E−02 | 1.66E−02 |
| β₅ | −3.63E+00 | −3.18E+00 | −6.27E+00 | −4.43E+00 |
| β₆ | 5.07E−01 | 7.07E−01 | 7.63E−01 | 1.04E+00 |
| β₇ | −2.02E+00 | −1.55E+00 | −2.76E+00 | −2.17E+00 |
| β₈ | 4.16E−02 | 5.45E−02 | 1.96E+00 | 2.40E+00 |
| β₉ | 7.87E+00 | 1.11E+01 | 6.62E+00 | 7.59E+00 |
| β₁₀ | −6.62E−02 | −4.65E−02 | −2.44E−01 | −1.74E−01 |
| β₁₁ | 1.28E−01 | 1.85E−01 | 4.57E−01 | 5.89E−01 |
| β₁₂ | −1.45E+00 | −1.01E+00 | −1.97E+00 | −1.53E+00 |

The spline terms of sp1(tPSA), sp2(tPSA), sp1(fPSA), and sp2(fPSA) in the model above may be determined according to the cubic spline formula presented above under model #7 above (Equations (10 and 11)). In some embodiments, the values of internal knots 2 and 3 and external knots 1 and 4 are within the ranges set forth in Table 3 below for tPSA and fPSA.

TABLE 3
Knot value ranges

| | | |
|---|---|---|
| Knot | | |
| value | Total PSA | Free PSA |

| ranges | Low | High | Low | High |
|---|---|---|---|---|
| Knot 1 | 0 | 2 | 0 | 0.5 |
| Knot 2 | 3.72E+00 | 4.16E+00 | 7.38E−01 | 9.43E−01 |
| Knot 3 | 4.71E+00 | 6.56E+00 | 1.10E+00 | 1.43E+00 |
| Knot 4 | 2.33E+02 | 3.13E+02 | 2.04E+01 | 2.78E+01 |

In some embodiments, a first logistic regression model may be used when a value for one or more of the proteins (markers) is above a certain threshold, and a second logistic regression model may be used when the value is below the threshold. A logistic regression model may be selected based on a threshold in accordance with some embodiments of the invention. As a non-limiting example, a value for the blood protein (marker) total PSA (tPSA) may be used to select a logistic regression model. protein (marker) However, it should be appreciated that any blood protein (marker) value, combination of blood protein (marker) values, or any other suitable information may alternatively be used. Accordingly, in some embodiments, at least one processor may be programmed to implement and select from a plurality of models based, at least in part, on one or more input values.

After receiving the one or more blood protein (marker) values, a logistic regression model may be selected selected based, at least in part, on the received blood protein (marker) value(s). For example, in one implementation, when the value of tPSA is ≥15 ng/ml, preferably ≥20 ng/ml and most preferably ≥25 ng/ml, the logistic regression model may be based on tPSA alone (e.g., the “Simple Model (tPSA only)” model described above may be used). For this implementation, when the tPSA value is less than a particular threshold (e.g., less than 15 ng/ml), one or more of the other logistic regression models may be selected.

After a model has been selected, it may be determined whether the selected model is a full model (e.g., includes all kallikrein proteins (markers) described herein) or is a partial model that includes less than all proteins (markers) in a kallikrein panel. If it is determined that the selected model is not a full model, the probability of cancer may be determined based solely on the received tPSA value, as described above. If it is determined that the selected model is a full model, the probability of cancer may be determined based on the selected model using multiple blood markers. Regardless of the particular model that is selected, after the probability of cancer is determined, the probability of cancer is output.

In some embodiments of the invention, said event for which said probability is obtained is evidence of prostate cancer at prostate biopsy taken from an asymptomatic subject or a subject with lower urinary tract symptoms.

In some embodiments of the invention, the event for which said probability is obtained is evidence of high grade prostate cancer, i.e., Gleason score 7 or higher, at prostate biopsy taken from an asymptomatic subject or a subject with lower urinary tract symptoms. Typically, the progression of prostate cancer or the prostate cancer status, is defined as (i) Gleason score 7 or higher, (ii) Gleason grade 4+3 or higher, or (iii) Gleason score 8 or higher.

In many preferred embodiments the data of the multitude of subjects comprises one or more biopsy data selected from the group consisting of reason for biopsy, year of biopsy, number of biopsy cores, the number of positive cores, the percent of positive in each core and any possible combination thereof.

As discussed above, in many preferred embodiments, the blood proteins (markers) are included in a logistic regression model employing up to two non-linear terms for at least one blood marker. In certain embodiments, the blood proteins (markers) are included in a logistic regression model employing up to three non-linear terms for at least one blood marker. In certain embodiments, the blood proteins (markers) are included in a logistic regression model employing up to four non-linear terms for at least one blood marker. In certain embodiments, the blood proteins (markers) are included in a logistic regression model including up to five non-linear terms for at least one blood protein (marker)

In some embodiments, the logistic regression model may be recalibrated when the anticipated event rate in a target population representative of the subject for which the event probability is to be obtained differs from the event rate of the multitude of subjects for which data have been employed to obtain the logistic regression model by defining, according to equation (II):

$$k = ( \frac{P / ( 1 - P )}{p / ( 1 - p )} ) , \qquad ( II )$$

wherein p is the event rate in said data of said multitude of subjects, and P is the anticipated event rate in said target population, defining, according to equation (III):

$$Odds = \frac{π}{1 - π} , \qquad ( III )$$

wherein π is the original probability from the model, and defining, according to equation (IV):

Odds_{recalibrated}=Odds×k (IV), and

obtaining a recalibrated probability, according to formula (V):

$${π}_{recalibrated} = ( \frac{{Odds}_{recalibrated}}{1 + {Odds}_{recalibrated}} ) , \qquad ( V )$$

wherein π_{recalibrated} is the probability of said event.

Some embodiments are directed to methods and apparatus for predicting prostate gland volume using a linear regression model, wherein said method comprises an act of a) providing a linear regression model obtained by employing linear regression of data of a multitude of subjects, said data comprising for each subject of said multitude of subjects: (i) data on prostate gland volume, and (ii) data, preceding data on prostate gland volume, comprising age; and determinations of blood proteins (markers) including one or more of tPSA, fPSA, iPSA, and optionally hK2 (free or total hK2), from blood samples of said subjects. In certain embodiments, the determinations of blood proteins include one or more of tPSA, fPSA, and optionally hK2 (free or total hK2) from blood samples of said subjects. In certain embodiments, the determination of blood proteins does not include iPSA. Said linear regression model may be generated employing formula (VI):

$$V = {\sum }_{i = 1}^{j} {β}_{i} {x}_{i} + c , \qquad ( VI )$$

wherein V is prostate gland volume, βᵢ is the coefficient for variable xᵢ for j variables comprising age, tPSA, fPSA, and optionally iPSA and/or hK2 (free or total hK2), respectively, to obtain said linear regression model. The method further comprises an act of b) providing the age of a subject in years, c) determining said blood proteins (markers) tPSA, fPSA, and optionally, iPSA and/or hK2 (free or total hK2), respectively, from a blood sample of said subject, and d) employing said linear regression model using said provided age of step b) and said determined blood proteins (markers) of step c) to obtain said predicted prostate volume of said subject. In some embodiments, the statistical model said risk for cancer is based on tPSA alone if tPSA is ≥15 ng/ml, preferably ≥20 ng/ml, and most preferably ≥25 ng/ml.

It should be appreciated that any suitable logistic regression model including, but not limited to, the models described above for determining a probability of prostate cancer upon biopsy, may be used with embodiments of the invention for determining prostate gland volume.

In some embodiments, the data of step a) (ii) for providing the logistic regression model or the linear regression model, and the determination of blood proteins (markers) of said subject comprise human kallikrein 2 (free or total hK2).

In many preferred embodiments of the method of the invention where prostate gland volume is predicted prostate gland volume is provided as defined by transrectal ultrasound.

In many preferred embodiments of the method of the present invention the data for each subject of said multitude of subjects for providing the logistic regression model or linear regression model further includes results of digital rectal examination (DRE) and accordingly DRE is carried out for the subject and obtained result is used when employing the logistic regression model or linear regression model, respectively, to obtain said probability. Preferably the results of DRE are expressed as binary values, i.e., normal=0, and nodularity present=1 with or without a second value for estimate volume, i.e., small=0, medium=1 and large=2.

In some preferred embodiments of the method of the present invention the data of the multitude of subjects for obtaining the model only comprises data of subjects with elevated levels, defined as age-specific median or higher, of tPSA and accordingly probabilities of the event or the predicted prostate volume are obtained only for subjects with said elevated levels of tPSA.

In preferred embodiments of the method of the present invention determinations of blood proteins (markers) of for each subject of the multitude of subjects for obtaining the model and accordingly those blood proteins (markers) determined to obtain the probability or predicted prostate gland volume are determined from blood samples of serum or plasma, preferably anti-coagulated, either fresh or frozen. Preferably all samples are of the same kind, i.e., either serum or plasma and either fresh or frozen.

In some preferred embodiments of the method of the present invention the logistic regression model or the linear regression model is provided employing data of a multitude of subjects aged 40 to 75 years; and accordingly the probability of the event or the predicted prostate volume is obtained of a subject aged 40 to 75 years.

In some preferred embodiments the method of the present invention the logistic regression model or the linear regression model is provided employing data of a multitude of subjects with a tPSA in blood ≥top age tertile, ≥top age quartile, ≥top age quintile, or ≥top age decile, and accordingly the probability of the event or the predicted prostate volume is obtained of a subject with tPSA in blood ≥top age tertile, ≥top age quartile, ≥top age quintile, or ≥top age decile, respectively. As an example, for a subject of age sixty, the corresponding total PSA values may be: 1.5 ng/ml, for the ≥top age tertile, 1.9 ng/ml, for the ≥top age quartile, 2.1 ng/ml, for the ≥top age quintile, and 3 ng/ml, for the ≥top age decile.

Computer Implementation

An illustrative implementation of a computer system on which some or all of the techniques and/or user interactions described herein may be may include one or more processors and one or more computer-readable non-transitory storage media (e.g., memory and one or more non-volatile storage media). The processor(s) may control writing data to and reading data from the memory and/or the non-volatile storage device in any suitable manner, as the aspects of the present invention described herein are not limited in this respect.

To perform any of the functionality described herein, the processor(s) may execute one or more instructions, such as program modules, stored in one or more computer-readable storage media (e.g., the memory), which may serve as non-transitory computer-readable storage media storing instructions for execution by the processor. Generally, program modules include routines, programs, objects, components, data structures, etc. that perform particular tasks or implement particular abstract data types. Embodiments may also be implemented in distributed computing environments where tasks are performed by remote processing devices that are linked through a communications network. In a distributed computing environment, program modules may be located in both local and remote computer storage media including memory storage devices.

The computer contemplated for use herein may operate in a networked environment using logical connections to one or more remote computers. The one or more remote computers may include a personal computer, a server, a router, a network PC, a peer device or other common network node, and may include many or all of the elements described above relative to the computer. Logical connections between the computer and the one or more remote computers may include, but are not limited to, a local area network (LAN) and a wide area network (WAN), but may also include other networks known or contemplated in the art. Such networks may be based on any suitable technology and may operate according to any suitable protocol and may include wireless networks, wired networks or fiber optic networks. Such networking environments are commonplace in offices, enterprise-wide computer networks, intranets and the Internet.

When used in a LAN networking environment, the computer may be connected to the LAN through a network interface or adapter. When used in a WAN networking environment, the computer typically includes a modem or other means for establishing communications over the WAN, such as the Internet. In a networked environment, program modules, or portions thereof, may be stored in the remote memory storage device.

Various inputs described herein for assessing a risk of prostate cancer and/or determining a prostate gland volume may be received by a computer via a network (e.g., a LAN, a WAN, or some other network) from one or more remote computers or devices that stores data associated with the inputs. One or more of the remote computers/devices may perform analysis on remotely-stored data prior to sending analysis results as the input data to a computer. Alternatively, the remotely stored data may be sent to a computer as it was stored remotely (i.e., providing the remotely stored data without any remote analysis). Additionally, inputs may be received directly by a user of a computer using any of a number of input interfaces (e.g., an input interface) that may be incorporated as components of a computer.

Various outputs described herein, including output of a probability of prostate cancer risk and/or prostate gland volume, may be provided visually on an output device (e.g., a display) connected directly to a computer or the output(s) may be provided to a remotely-located output device connected to a computer via one or more wired or wireless networks, as embodiments of the invention are not limited in this respect. Outputs described herein may additionally or alternatively be provided other than using visual presentation. For example, a computer or a remote computer to which an output is provided may include one or more output interfaces including, but not limited to speakers, and vibratory output interfaces, for providing an indication of the output.

It should be appreciated that although a computer may be a single device, in some embodiments, the computer may comprise a plurality of devices communicatively coupled to perform some or all of the functionality described herein.

As described above, in some embodiments, the computer contemplated for use herein may be included in a networked environment, where information about one or more blood markers, used to determine a probability of prostate cancer and/or prostate gland volume, is sent from an external source to the computer for analysis using one or more of the techniques described herein. For example, a networked environment may be used. In the contemplated network environment, the computer may be connected to one or more detectors via a network. As discussed above, the network may be any suitable type of wired or wireless network, and may include one or more local area networks (LANs) or wide area networks (WANs), such as the Internet.

The detector may be configured to determine values for one or more of the blood proteins (markers) used to determine a probability of prostate cancer and/or prostate gland volume, in accordance with one or more of the techniques described herein. Although the detector may be used alone (i.e., as a single detector, it should be appreciated that the detector may also be used with other detectors (i.e., as a group of detectors), with each detector configured to determine one or more of the blood protein (marker) values used in accordance with one or more of the techniques described herein.

In some embodiments, information corresponding to the values for the blood proteins (markers) determined from the detector may be stored prior to sending the values to a computer. In such embodiments, the information corresponding to the values may be stored locally in a local storage device communicatively coupled to a detector and/or stored in a network-connected central storage device. Accordingly, when values corresponding to the blood proteins (markers) are received by the computer in accordance with one or more of the techniques described herein, it should be appreciated that at least some of the values may be received directly from the detector or from one or more storage devices (e.g., one or more local storage devices or central storage devices) on which the values have been stored, as embodiments are not limited based on where the values are received from or stored.

The calculation methods, steps, simulations, algorithms, systems, and system elements described herein may be implemented using a computer system, such as the various embodiments of computer systems described below. The methods, steps, systems, and system elements described herein are not limited in their implementation to any specific computer system described herein, as many other different machines may be used.

The computer implemented control system can be part of or coupled in operative association with a sample analyzer, and, in some embodiments, configured and/or programmed to control and adjust operational parameters of the sample analyzer, as well as analyze and calculate values, as described above. In some embodiments, the computer implemented control system can send and receive reference signals to set and/or control operating parameters of the sample analyzer and, optionally, other system apparatus. In other embodiments, the computer implemented system can be separate from and/or remotely located with respect to the sample analyzer and may be configured to receive data from one or more remote sample analyzer apparatus via indirect and/or portable means, such as via portable electronic data storage devices, such as magnetic disks, or via communication over a computer network, such as the Internet or a local intranet.

The computer implemented control system may include several known components and circuitry, including a processing unit (i.e., a processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system may be a multi-processor computer system or may include multiple computers connected over a computer network.

The computer system may include a processor, for example, a commercially available processor such as one of the series x86, Celeron and Pentium processors (including the Pentium IV processor), available from Intel, similar devices from AMD and Cyrix, the 680X0 series microprocessors available from Motorola, the PowerPC microprocessor from IBM, and ARM processors. Many other processors are available, and the computer system is not limited to a particular processor.

A processor typically executes a program called an operating system, of which WindowsNT, Windows 2000, Windows95 or 98, Windows 7, Windows 8, UNIX, Linux, DOS, VMS, MacOS and OSX, and iOS are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer system is not limited to a particular computer platform.

The computer system may include a memory system, which typically includes a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive.

Such a recording medium stores signals, typically in binary form (i.e., a form interpreted as a sequence of one and zeros). A disk (e.g., magnetic or optical) has a number of tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer system also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer system that implements the methods, steps, systems and system elements described above is not limited thereto. The computer system is not limited to a particular memory system.

At least part of such a memory system described above may be used to store one or more data structures (e.g., look-up tables) or equations described above. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer system may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer system may include one or more output devices. Example output devices include a cathode ray tube (CRT) display, liquid crystal displays (LCD) and other video output devices, printers, communication devices such as a modem or network interface, storage devices such as disk or tape, and audio output devices such as a speaker.

The computer system also may include one or more input devices. Example input devices include a keyboard, keypad, track ball, mouse, pen and tablet, communication devices such as described above, and data input devices such as audio and video capture devices and sensors. The computer system is not limited to the particular input or output devices described herein.

It should be appreciated that one or more of any type of computer system may be used to implement various embodiments described herein. Aspects of the disclosure may be implemented in software, hardware or firmware, or any combination thereof. The computer system may include specially programmed, special purpose hardware, for example, an application-specific integrated circuit (ASIC). Such special-purpose hardware may be configured to implement one or more of the methods, steps, simulations, algorithms, systems, and system elements described above as part of the computer system described above or as an independent component.

The computer system and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

The methods, steps, simulations, algorithms, systems, and system elements may be implemented using any of a variety of suitable programming languages, including procedural programming languages, object-oriented programming languages, other languages and combinations thereof, which may be executed by such a computer system. Such methods, steps, simulations, algorithms, systems, and system elements can be implemented as separate modules of a computer program, or can be implemented individually as separate computer programs. Such modules and programs can be executed on separate computers.

Such methods, steps, simulations, algorithms, systems, and system elements, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system, or system element, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system, or system element.

It should be appreciated that various embodiments may be formed with one or more of the above-described features. The above aspects and features may be employed in any suitable combination as the present invention is not limited in this respect. It should also be appreciated that the drawings illustrate various components and features which may be incorporated into various embodiments. For simplification, some of the drawings may illustrate more than one optional feature or component. However, the invention is not limited to the specific embodiments disclosed in the drawings. It should be recognized that the disclosure encompasses embodiments which may include only a portion of the components illustrated in any one drawing figure, and/or may also encompass embodiments combining components illustrated in multiple different drawing figures.

### Other Systems and Components

As described herein, in some embodiments, a system may include a processor or computer programmed to evaluate a logistic regression model in electronic communication with an analyzer for determining a probability of an event associated with prostate cancer (e.g., risk of prostate cancer and/or prostate gland volume). The analyzer may be adapted and arranged to determine one or more characteristics of blood proteins (markers) for inputting into the logistic regression model. In some embodiments, the analyzer is a fluorescence laser scanner. In certain embodiments, the analyzer may be adapted and arranged to analyze a sample present on one or more solid supports (e.g., on one or more chips). For example, an adapter may be used so that two or more solid supports may be inserted into the analyzer at the same time. As a non-limiting example, the adapter may be a 2-slide adapter, a 3-slide adapter, a 4-slide adapter, a 5-slide adapter, or a 6-slide adapter. As a non-limiting example, the adapter may be a 2-chip adapter, a 3-chip adapter, a 4-chip adapter, a 5-chip adapter, or a 6-chip adapter. It should be appreciated, however, that other types of analyzers may also be used (e.g., analyzers for microwell ELISA-type assays) and that the systems described herein are not limited in this respect.

An example of such a system includes, in one set of embodiments, an analyzer comprising a housing, an opening in the housing configured to receive one or more chips or a chip adapter, wherein the housing includes a component configured to interface with a mating component on the one or more chips or a chip adapter to detect the one or more chips or a chip adapter within the housing. The analyzer may further comprise an optical system positioned within the housing, the optical system including at least one light source and at least one detector spaced adjacent to or apart from the light source, wherein the light source is configured to illuminate the one or more chips when they are inserted into the sample analyzer and wherein the detector is positioned to detect the amount of light that emits from or passes through the one or more chips. The system may also include a user interface associated with the housing for inputting at least the age of a person and/or other information for inputting into the linear regression model.

In certain embodiments, a processor is (or is adapted to be) in electronic communication with the analyzer. In some cases, the processor is within the housing of the analyzer. However, in other embodiments, the processor is not included within the housing of the analyzer but may be accessed by electronic means as described herein. The processor may be programmed to evaluate a logistic regression model based, at least in part, on information received from the at least one detector to determine a probability of an event associated with prostate cancer in a person, wherein evaluating the logistic regression model comprises scaling each of a plurality of variables by a different coefficient value to produce scaled variables and summing values for the scaled variables used to produce the probability of the event associated with prostate cancer in a person, wherein the plurality of variables includes age and at least two variables included in the information received from the detector and is selected from the group consisting of fPSA, iPSA, tPSA, total hK2, and/or free hK2. In some embodiments, the at least two variables included in the information received from the detector do not include data about, levels of, or information about iPSA.

A method for determining a probability of an event associated with prostate cancer in a person may include, for example, providing an analyzer. The analyzer may comprise a housing, an opening in the housing configured to receive one or more chips or a chip adapter, wherein the housing includes a component configured to interface with a mating component on the one or more chips or a chip adapter to detect the one or more chips or a chip adapter within the housing. The analyzer may further include an optical system positioned within the housing, the optical system including at least one light source and at least one detector spaced adjacent to or apart from the light source, wherein the light source is configured to illuminate the one or more chips or a chip adapter when the one or more chips or a chip adapter is inserted into the sample analyzer and wherein the detector is positioned to detect the amount of light that emits from or passes through the one or more chips. The analyzer may also include a user interface associated with the housing for inputting at least the age of a person. The method may involve determining information for a plurality of blood proteins (markers) using the analyzer, wherein the information for the plurality of blood proteins (markers) includes a fPSA value, iPSA value, tPSA value, a free hK2 value, and/or a total hK2 value. The method may involve determining information for a plurality of blood proteins (markers) using the analyzer, wherein the information for the plurality of blood proteins (markers) includes a fPSA value, tPSA value, a free hK2 value, and/or a total hK2 value. In certain embodiments, the information for the plurality of blood proteins (markers) does not include an iPSA value. The method may also involve evaluating, using at least one processor, a logistic regression model based, at least in part, on the information to determine a probability of an event associated with prostate cancer in a person, wherein evaluating the logistic regression model comprises scaling each of a plurality of variables by a different coefficient value to produce scaled variables and summing values for the scaled variables used to produce the probability of the event associated with prostate cancer in a person, wherein the plurality of variables includes age and at least two variables included in the information received from the detector and is selected from the group consisting of fPSA, iPSA, tPSA, free hK2, and/or total hK2. In certain embodiments, the at least two variables does not include iPSA.

In one set of embodiments, the system includes a device (e.g., a chip) comprising a first liquid containment region comprising a first binding partner and a second binding partner. Another example of a system includes, in one set of embodiments, a device (e.g., a chip) comprising a first liquid containment region comprising a first binding partner and a second liquid containment region comprising a second binding partner. The first binding partner is adapted to bind with at least one of fPSA, iPSA, tPSA, free hK2, and/or total hK2, and the second binding partner is adapted to bind with at least another of fPSA, iPSA, tPSA, free hK2, and/or total hK2. In some embodiments, the device includes a third liquid containment region including a third binding partner adapted to bind with the third of fPSA, iPSA, tPSA, free hK2, and/or total hK2. In some embodiments, the device includes a fourth liquid containment region including a fourth binding partner adapted to bind with the third of fPSA, iPSA, tPSA, free hK2, and/or total hK2. In some embodiments, neither or none of the binding partners are adapted to bind with iPSA. The system includes a detector associated with the first and second liquid containment region, and a processor programmed to evaluate a logistic regression model based, at least in part, on information received from the detector to determine a probability of an event associated with prostate cancer in a person. Evaluating the logistic regression model comprises scaling each of a plurality of variables by a different coefficient value to produce scaled variables and summing values for the scaled variables used to produce the probability of the event associated with prostate cancer in a person, wherein the plurality of variables includes age and at least two variables included in the information received from the detector and is selected from the group consisting of fPSA, iPSA, tPSA, free hK2, and/or total hK2. In some embodiments, the plurality of variables does not include iPSA. In certain embodiments, there are additional detectors associated with the additional liquid containment regions.

A method of determining the probability of the event associated with prostate cancer in such a system may include, for example, the acts of introducing a sample into or onto a device (e.g., a chip) comprising a first liquid containment region comprising a first binding partner and a second liquid containment region comprising a second binding partner, wherein the first binding partner is adapted to bind with at least one of fPSA, iPSA, tPSA, free hK2, and/or total hK2, and wherein the second binding partner is adapted to bind with at least another of fPSA, iPSA, tPSA, free hK2, and/or total hK2. In some embodiments, the device includes a third liquid containment region including a third binding partner adapted to bind with the third of fPSA, iPSA, tPSA, free hK2, and/or total hK2. In some embodiments, the device includes a fourth liquid containment region including a fourth binding partner adapted to bind with the third of fPSA, iPSA, tPSA, free hK2, and/or total hK2. The method may involve allowing any of the fPSA, iPSA, tPSA, free hK2, and/or total hK2 from the sample to bind with at least the first and/or second binding partners at the first and second liquid containment regions and determining a characteristic of fPSA, iPSA, tPSA, free hK2, and/or total hK2 using one or more detectors associated with the first and second liquid containment regions. In some embodiments, neither or none of the binding partners are adapted to bind with iPSA.

Another method of determining the probability of the event associated with prostate cancer in such a system may include, for example, the acts of introducing a sample into or onto a device (e.g., a chip) comprising a first liquid containment region comprising a first binding partner and a second binding partner, wherein the first binding partner is adapted to bind with at least one of fPSA, tPSA, free hK2, and/or total hK2, and wherein the second binding partner is adapted to bind with at least another of fPSA, tPSA, free hK2, and/or total hK2. In some embodiments, the device includes a third liquid containment region including a third binding partner adapted to bind with the third of fPSA, tPSA, free hK2, and/or total hK2. In some embodiments, the device includes a fourth liquid containment region including a fourth binding partner adapted to bind with the third of fPSA, tPSA, free hK2, and/or total hK2. The method may involve allowing any of the fPSA, tPSA, free hK2, and/or total hK2 from the sample to bind with at least the first and/or second binding partners at the first and/or second liquid containment regions and determining a characteristic of fPSA, tPSA, free hK2, and/or total hK2 using one or more detectors associated with the first and/or second liquid containment regions.

The method involves inputting the characteristics of fPSA, iPSA, tPSA, free hK2, and/or total hK2 into a processor programmed to evaluate a logistic regression model based, at least in part, on information received from the at least one detector to determine a probability of an event associated with prostate cancer in a person, wherein evaluating the logistic regression model comprises scaling each of a plurality of variables by a different coefficient value to produce scaled variables and summing values for the scaled variables used to produce the probability of the event associated with prostate cancer in a person, wherein the plurality of variables includes age and at least two variables included in the information received from the detector and is selected from the group consisting of fPSA, iPSA, tPSA, free hK2, and/or total hK2. Accordingly, the probability of the event associated with prostate cancer may be determined. The method may also involve inputting the characteristics of fPSA, tPSA, free hK2, and/or total hK2 into a processor programmed to evaluate a logistic regression model based, at least in part, on information received from the at least one detector to determine a probability of an event associated with prostate cancer in a person, wherein evaluating the logistic regression model comprises scaling each of a plurality of variables by a different coefficient value to produce scaled variables and summing values for the scaled variables used to produce the probability of the event associated with prostate cancer in a person, wherein the plurality of variables includes age and at least two variables included in the information received from the detector and is selected from the group consisting of fPSA, tPSA, free hK2, and/or total hK2. In some embodiments, the method does not include the use of information or data about iPSA. Accordingly, the probability of the event associated with prostate cancer may be determined.

In certain embodiments, a device for determining blood proteins (markers) (e.g., fPSA, iPSA, tPSA, free hK2, and/or total hK2) is provided. In some embodiments, the device is or may not be used for determining information about iPSA. In some cases, the device may allow for simultaneous determination of the blood markers, e.g., on a single chip. The device may include one or more liquid containment region, and each of the liquid containment regions may have one or more analysis regions. For example, the device may include a single liquid containment region with one or more analysis regions. The device may include two or more liquid containment region, and each of the liquid containment regions may have one or more analysis regions. Each of the analysis regions may include one of an anti-iPSA specific capture antibody, an anti-fPSA specific capture antibody, an anti-tPSA specific capture antibody, a free hK2 specific capture antibody, and/or a total hK2 specific capture antibody). In some embodiments, none of the analysis regions include an anti-iPSA specific capture antibody. Two or more of the liquid containment regions are not in fluid communication and are considered “fluidically isolated”.

Certain reagents may be attached to the first, second, third and/or fourth liquid containment regions, e.g., prior to use of the device. The reagents may include, for example, an anti-iPSA specific capture antibody, an anti-fPSA specific capture antibody, an anti-tPSA specific capture antibody, and/or an hK2 (free or total) specific capture antibody). In certain embodiments, only one liquid containment region is present in the device. In some embodiments, the reagents do not include an anti-iPSA specific capture antibody.

Two different detector antibodies, e.g., an anti-tPSA detector antibody with a fluorescent tag for one wavelength, and an anti-fPSA detector antibody with a fluorescent tag for a different wavelength, may be used for detection. A different analysis region may include, for example, an anti-fPSA capture antibody, and optionally an anti-iPSA capture antibody. In some embodiments, the analysis region does not include an anti-iPSA capture antibody. Two different detector antibodies, e.g., an anti-fPSA detector antibody with a fluorescent tag for one wavelength, and an anti-iPSA detector antibody with a fluorescent tag for a different wavelength, may be used for detection. In certain embodiments, specific capture antibodies may be used for detection of the species. In some embodiments, an anti-iPSA detector antibody is not used for detection.

In certain embodiments, an identification system including one or more identifiers is used and associated with one or more components or materials associated with a solid support (e.g., a chip) and/or analyzer. The “identifiers,” as described in greater detail below, may themselves be “encoded with” information (i.e., carry or contain information, such as by use of an information carrying, storing, generating, or conveying device such as a radio frequency identification (RFID) tag or barcode) about the component including the identifier, or may not themselves be encoded with information about the component, but rather may only be associated with information that may be contained in, for example, a database on a computer or on a computer readable medium (e.g., information about a user, and/or sample to be analyzed). In the latter instance, detection of such an identifier can trigger retrieval and usage of the associated information from the database.

Identifiers “encoded with” information about a component need not necessarily be encoded with a complete set of information about the component. For example, in certain embodiments, an identifier may be encoded with information merely sufficient to enable a unique identification of the one or more chips or a chip adapter (e.g., relating to a serial no., part no., etc.), while additional information relating to the one or more chips or a chip adapter (e.g., type, use (e.g., type of assay), ownership, location, position, connectivity, contents, etc.) may be stored remotely and be only associated with the identifier.

“Information about” or “information associated with” a solid support (e.g., a chip), material, or component, etc., is information regarding the identity, positioning, or location of the solid support (e.g., a chip), material or component or the identity, positioning, or location of the contents of a solid support (e.g., a chip), material or component and may additionally include information regarding the nature, state or composition of the solid support (e.g., a chip), material, component or contents. “Information about” or “information associated with” a solid support (e.g., a chip), material or component or its contents can include information identifying the solid support (e.g., a chip), material or component or its contents and distinguishing the solid support (e.g., a chip), material, component or its contents from others. For example, “information about” or “information associated with” a solid support (e.g., a chip), material or component or its contents may refer to information indicating the type or what the chip, material or component or its contents is, where it is or should be located, how it is or should be positioned, the function or purpose of the solid support (e.g., a chip), material or component or its contents, how the solid support (e.g., a chip), material or component or its contents is to be connected with other components of the system, the lot number, origin, calibration information, expiration date, destination, manufacturer or ownership of the solid support (e.g., a chip), material or component or its contents, the type of analysis/assay to be performed in or on the chip, information about whether the chip has been used/analyzed, etc.

Non-limiting examples of identifiers that may be used in the context of the invention include radio frequency identification (RFID) tags, barcodes, serial numbers, color tags, fluorescent or optical tags (e.g., using quantum dots), chemical compounds, radio tags, magnetic tags, among others.

In one embodiment, the identifier of a chip may be associated with predetermined or programmed information contained in a database regarding the use of the system or chip for a particular purpose, user or product, or with particular reaction conditions, sample types, reagents, users, and the like. If an incorrect match is detected or an identifier has been deactivated, the process may be halted or the system may be rendered not operable until the user has been notified, or upon acknowledgement by a user.

It should be appreciated that various embodiments may be formed with one or more of the above-described features. The above aspects and features may be employed in any suitable combination as the present disclosure is not limited in this respect. It should also be appreciated that the drawings illustrate various components and features which may be incorporated into various embodiments. For simplification, some of the drawings may illustrate more than one optional feature or component. However, it should be recognized that the disclosure encompasses embodiments which may include only a portion of the components illustrated in any one drawing figure, and/or may also encompass embodiments combining components illustrated in multiple different drawing figures.

The embodiments and methods described herein may be combined with one or more embodiments and/or methods described in U.S. application Ser. No. 14/671,355, filed on Mar. 27, 2015 and entitled “Compositions and Methods Related to Diagnosis of Prostate Cancer”, which is hereby incorporated by reference in its entirety for all purposes.

### Examples

### Example 1—Assay and Predictive Model

Described herein is an assay based on a panel of four kallikrein proteins (markers) that include total prostate specific antigen (tPSA), free PSA (fPSA), intact PSA (iPSA), and human Kallikrein 2 (hK2) linked to subject specific information. The four kallikrein proteins (markers) have been studied individually and in various combinations for prostate cancer detection applications. A logistic regression algorithm incorporating the blood plasma levels of these four proteins (markers) as well as subject-specific information such as age, result from a digital rectal exam (DRE) and existence of prior negative prostate biopsy(-ies) demonstrate a higher positive predictive value for prostate cancer than the PSA test alone.

Levels (e.g., in ng/mL) of tPSA, fPSA, iPSA, and hK2 present in human plasma samples are determined using the AutoDELFIA automatic immunoassay system. The averaged amount of each protein (marker) is calculated from the duplicate tests for each protein (marker) and used in a predictive model to determine a risk score for a given human plasma sample. tPSA and fPSA may also be determined using an Elecsys immunoassay analyzer (Roche Diagnostics).

Each run uses at least one set of two plates—one plate for free/total PSA and one plate for iPSA. hK2 can be analyzed using either of the plates. A complete run at full capacity involves two sets of these two plates. The whole procedure involves approximately 3 to 5 hours from the initiation to obtaining the test results depending on the number of plates being run.

### Example 2—Immunoassay Chip

Described herein is an immunoassay based on a panel of four kallikrein proteins (markers) that include total prostate specific antigen (tPSA), free PSA (fPSA), intact PSA (iPSA), and human Kallikrein 2.

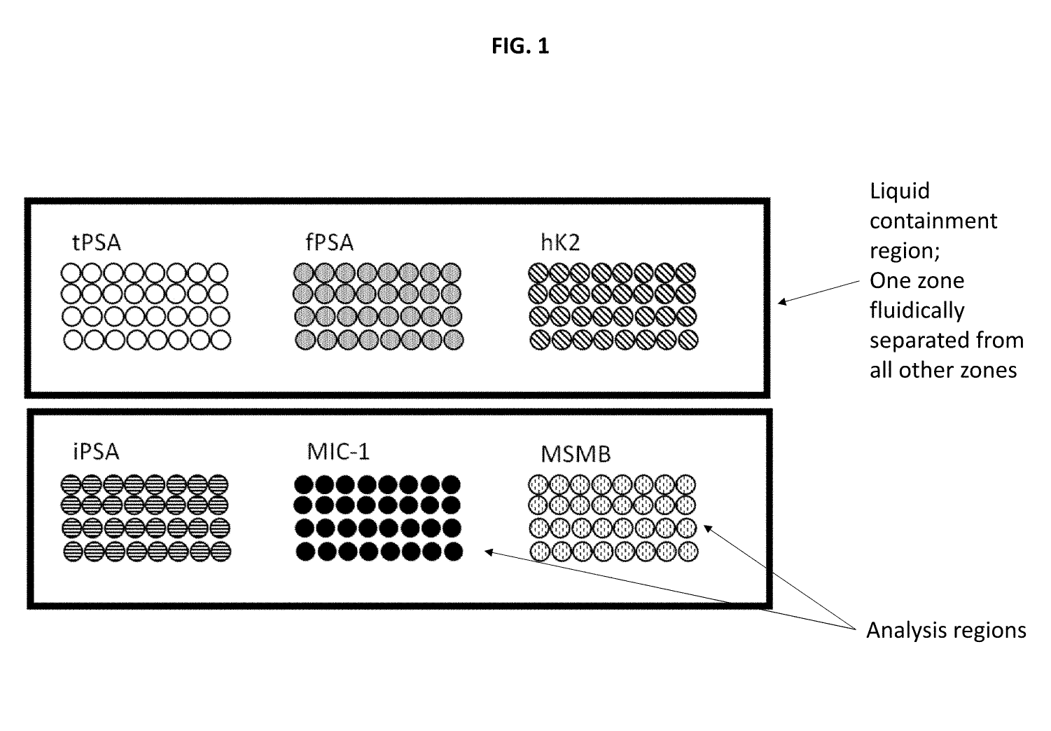

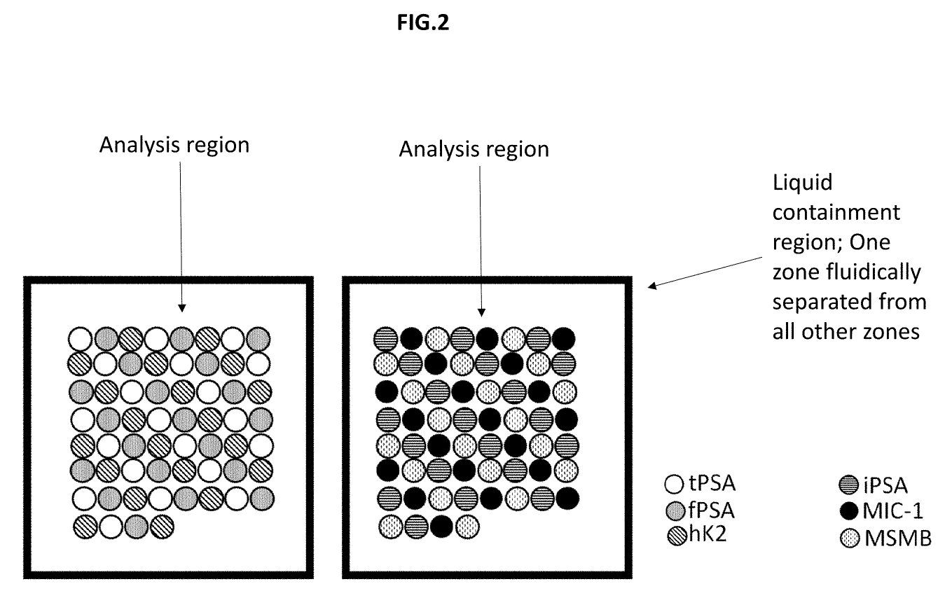

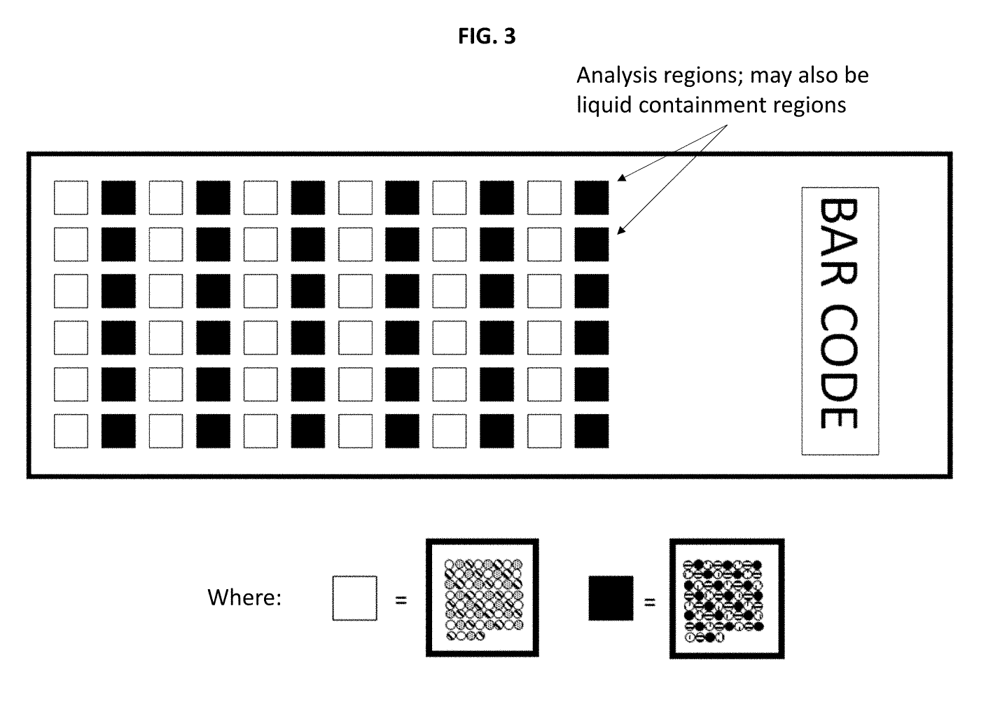

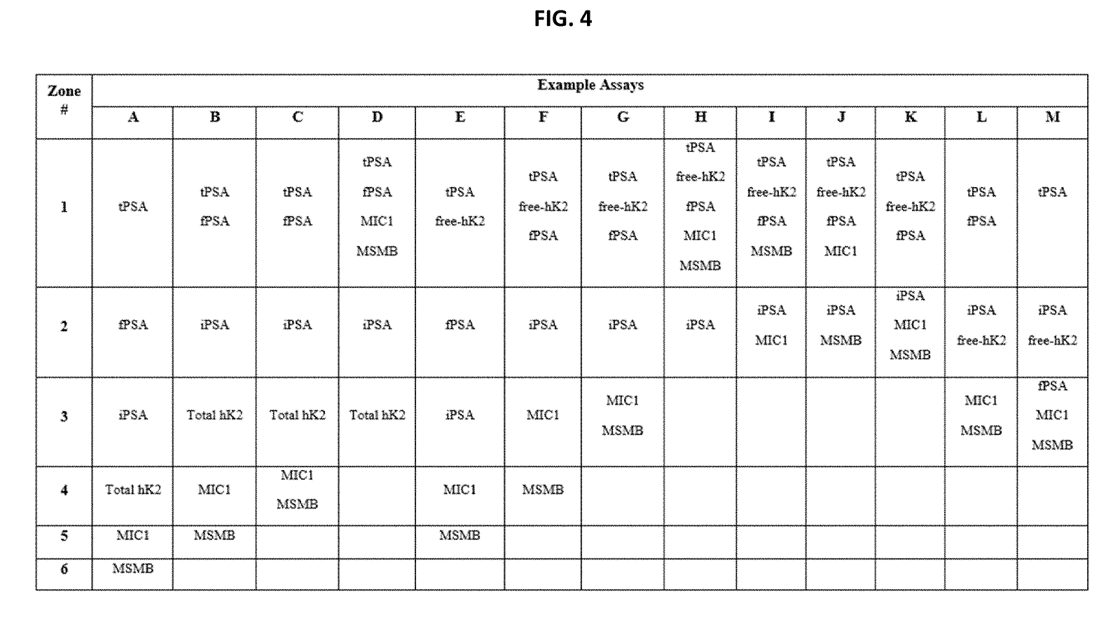

An immunoassay as described herein may be on a chip or slide that is made of a transparent material, such as glass. The immunoassay has two or more liquid containment regions that are not in fluid communication (i.e., are fluidically isolated). Each liquid containment region has one or more analysis regions with binding proteins that recognize one or more kallikrein proteins, represented in FIGS. 1 and 2 as circles. Binding proteins for kallikrein proteins with common epitopes are segregated into different liquid containment regions. Binding proteins for kallikrein proteins with unique epitopes may be in the same liquid containment regions. The different binding proteins may be spotted as replicates in a group (as shown in FIG. 1), or may be spotted in an interspersed manner (as shown in FIG. 2). As another example, FIG. 3 shows a chip that has 96 (6×12) liquid containment regions, with each liquid containment region defined as a fluidically independent zone with a group of spots. For example, one group of spots could be a grid of 60 analysis regions or spots. Each grid of spots could, for example, have binding partners for different proteins. The proteins (markers) that will bind to the binding partners present in each liquid containment region should be selected so that they do not have common epitopes. In some embodiment, the binding partners present in each liquid containment region should be selected so that each binding partner binds to an epitope that is unique to one protein being detected in the liquid containment region (i.e., such that the epitope is not a common epitope shared with another protein being detected within the liquid containment region). Alternatively, in some embodiments, a set of binding partners should be used to detect a unique epitope for each protein being detected in the liquid containment region (e.g., the capture antibody and detection antibody used to identify the presence of a protein should detect the presence of a unique epitope on that protein, such that the unique epitope is not a common epitope shared with another protein being detected within that liquid containment region). As a specific example, one liquid containment region could have binding proteins for tPSA, fPSA, and free or total hK2; and a second liquid containment region could have binding proteins for iPSA, MIC-1, and MSMB. Exemplary assay combinations for use are shown in FIG. 4.

### Example 3—Exemplary Immunoassay A

Glass substrate (25×75 mm²) are thoroughly activated and cleaned with concentrated HCl, sequentially rinsed with deionized water, isopropanol, and high purity water (>18 MOhm cm) and dried with compressed nitrogen. The activated glass substrate are silanized in a 2% (v/v) of glycidoxypropyltrimethoxysilane in o-xylene (5 hours at 55° C.) with occasional mixing. The slides are rinsed with o-xylene, and dried at 135° C. A 2-mm thick Teflon mask with three 7×7 mm² openings is applied to the surface of the array, to prevent contact between reagents used in different openings. The capture probes (at a concentration of 2 mg/mL in phosphate buffer pH 8.4) are spotted with a commercial pin and ring instrument onto the surface of the glass, resulting in spots covering an area of approximately 0.2 mm in diameter. Three spots are prepared for each capture probe to generate triplicate measurements (for quality control procedure, see below). The capture probes for tPSA, fPSA, MIC1 and MSMB are spotted in a first opening, the capture probe for iPSA is spotted in the second opening, and the capture probe for total hK2 is spotted in a third opening. A series of controls (purified human IgE at increasing concentrations from 1 to 300 ug/mL) is spotted in each opening. The spots are left to dry in ambient conditions, and after 1 hour are rinsed with a blocking solution (5% milk powder in TRIS buffer pH 7 with 0.2% Tween-20 and 0.05% sodium azide). Subsequently, the blocking solution is rinsed off with a stabilizer solution (PBS pH7.4 with 1% sucrose and 0.05% sodium azide), and then left to dry at ambient conditions for three hours.

Prior to use of the microarray for analysis of a sample, the surface of the opening is rinsed first with a rinse solution (150 mM sodium chloride, 10 mM Tris base, and 0.5% Tween 20, pH 8.0), then rinsed with deionized water, and finally dried in ambient conditions. An undiluted serum sample is pipetted in the first opening (20 μL). The serum is diluted 1:1 (v:v) with an iPSA assay buffer (TRIS buffer pH 6.7 with BSA, denatured mouse IgG and commercial HAMA blockers) and pipetted in the second opening. The serum is separately diluted 1:1 (v:v) with hK2 assay buffer (TRIS buffer pH 7.5 with BSA, tPSA antibodies, denatured mouse IgG, commercial HAMA blockers) and pipetted in the third opening. The samples are incubated for three hours in an environmental chamber (relative humidity >90% and temperature 25° C.). The surface of the microarray is then rinsed first with a rinse solution, then rinsed with deionized water, and finally dried at ambient conditions. The tracer antibodies solutions are then pipetted in the three openings. The tracers for tPSA, fPSA, MIC1, and MSMB are pipetted in the first opening, the tracer for iPSA is pipetted in the second opening, and the tracer for total hK2 mixed with tPSA blockers is pipetted in the third opening. Each tracer solutions also contains an anti-human IgE tracer. All tracer reagents are conjugates of the fluorescent dye Cy3. The tracer solutions are incubated for one hour in an environmental chamber (relative humidity >90% and temperature 25 degree C.), then rinsed with rinse solution, dried and stored in the dark.

The microarray is analyzed with a commercial laser scanner to image each spot and selected control areas between the spots (to evaluate non-specific binding, NSB). NSB must be below a pre-defined threshold in order for the measurements to be considered accurate. A quantitative fluorescence measurement is established for each spot by subtracting the NSB from the raw (unsubtracted) intensity of each spot. Consistency in the intensity across the triplicate measurements is assessed across the microarray. A maximum of one replicate is removed if a CV across triplicates is greater than 10%, and if outlier removal would bring the CV to less than 10%. The series of IgE controls is used to establish a relationship between fluorescent intensity and protein concentration, and the concentrations of tPSA, fPSA, MIC1, MSMB, iPSA and hK2 are calculated.

### Example 4—Exemplary Immunoassay B

Glass substrate (25×75 mm²) are thoroughly activated and cleaned with concentrated HCl, sequentially rinsed with deionized water, isopropanol, and high purity water (>18 MOhm cm) and dried with compressed nitrogen. The activated glass substrate are silanized in a 2% (v/v) of glycidoxypropyltrimethoxysilane in o-xylene (5 hours at 55° C.) with occasional mixing. The slides are rinsed with o-xylene, and dried at 135° C. A 2-mm thick Teflon mask with three 7×7 mm² openings is applied to the surface of the array, to prevent contact between reagents used in different openings. The capture probes (at a concentration of 2 mg/mL in phosphate buffer pH 8.4) are spotted with a commercial pin and ring instrument onto the surface of the glass, resulting in spots covering an area of approximately 0.2 mm in diameter. Three spots are prepared for each capture probe to generate triplicate measurements (for quality control procedure, see below). The capture probes for tPSA, hK2, and fPSA are spotted in a first opening; and the capture probes for iPSA, MIC1, and MSMB are spotted in the second opening. A series of controls (purified human IgE at increasing concentrations from 1 to 300 ug/mL) is spotted in each opening. The spots are left to dry in ambient conditions, and after 1 hour are rinsed with a blocking solution (5% milk powder in TRIS buffer pH 7 with 0.2% Tween-20 and 0.05% sodium azide). Subsequently, the blocking solution is rinsed off with a stabilizer solution (PBS pH7.4 with 1% sucrose and 0.05% sodium azide), and then left to dry at ambient conditions for three hours.

Prior to use of the microarray for analysis of a sample, the surface of the openings are rinsed first with a rinse solution (150 mM sodium chloride, 10 mM Tris base, and 0.5% Tween 20, pH 8.0), then rinsed with deionized water, and finally dried in ambient conditions. An undiluted serum sample is pipetted in the first opening and the second opening (20 μL). The serum is diluted 1:1 (v:v) with appropriate buffer solutions. The samples are incubated for three hours in an environmental chamber (relative humidity >90% and temperature 25° C.). The surface of the microarray is then rinsed first with a rinse solution, then rinsed with deionized water, and finally dried at ambient conditions. The tracer antibodies solutions are then pipetted in the openings. The tracers for tPSA, hK2, and fPSA are spotted in the first opening; and the tracers for iPSA, MIC1, and MSMB are pipetted in the second opening. Each tracer solutions also contains an anti-human IgE tracer. All tracer reagents are labeled and may, for example, be conjugated to a fluorescent dye (for example, Cy3). The tracer solutions are incubated for one hour in an environmental chamber (relative humidity >90% and temperature 25 degree C.), then rinsed with rinse solution, dried and stored in the dark.

The microarray is analyzed with a commercial laser scanner to image each spot and selected control areas between the spots (to evaluate non-specific binding, NSB). NSB must be below a pre-defined threshold in order for the measurements to be considered accurate. A quantitative fluorescence measurement is established for each spot by subtracting the NSB from the raw (unsubtracted) intensity of each spot. Consistency in the intensity across the triplicate measurements is assessed across the microarray. A maximum of one replicate is removed if a CV across triplicates is greater than 10%, and if outlier removal would bring the CV to less than 10%. The series of IgE controls is used to establish a relationship between fluorescent intensity and protein concentration, and the concentrations of tPSA, fPSA, MIC1, MSMB, iPSA and hK2 are calculated.

### Example 5—Immunoassay and Model

Described herein is an assay based on a panel of four kallikrein proteins (markers) that include total prostate specific antigen (tPSA), free PSA (fPSA), intact PSA (iPSA), and human Kallikrein 2 (total hK2 or free hK2). The four kallikrein proteins (markers) have been studied individually and in various combinations for prostate cancer detection applications.

Levels (e.g., in ng/mL) of tPSA, fPSA, iPSA, free hK2 and/or total hK2 present in human plasma samples are determined. The levels of additional proteins (markers) such as MSMB and/or MIC-1 may also be determined using any method described herein. The averaged amount of each protein (marker) is calculated from the duplicate tests for each protein (marker) and subsequently analyzed using a predictive model to determine a risk score for a given human plasma sample. The level of any protein (marker) may also be determined using an analyzer such as an Elecsys immunoassay analyzer (Roche Diagnostics).

Each run uses at least one plate with two liquid containment regions, each of the liquid containment regions having one or more analysis region. The liquid containment regions may not be in fluidic communication (i.e., liquid cannot pass between the regions).

Additionally, genetic profile information may be gathered through, for example, levels of one or more SNPs. The SNPs may be any of the SNPs disclosed herein or known to be related to prostate cancer (including those SNPs generally known to be related to any proliferative disorder or risk factor for a proliferative disorder). Any number of SNPs can be chosen for this additional genetic analysis, and this selection of SNPs may be related to the population under examination. In certain embodiments, no SNPs are used and/or no additional genetic analysis is performed.

A logistic regression algorithm incorporating the blood plasma levels of recited proteins (markers) as well as subject-specific information such as age, results from a digital rectal exam (DRE), existence of prior negative prostate biopsy(-ies), and genetic profile information (such as that described above) may demonstrate a higher positive predictive value for prostate cancer than the PSA test alone.

### Example 6—Assay and Predictive Model

Described herein is an assay based on a panel of three kallikrein proteins (markers) that include total prostate specific antigen (tPSA), free PSA (fPSA), and human Kallikrein 2 (hK2) linked to subject specific information. The three kallikrein proteins (markers) have been studied individually and in various combinations for prostate cancer detection applications. A logistic regression algorithm incorporating the blood plasma levels of these three proteins (markers) as well as subject-specific information such as age, result from a digital rectal exam (DRE) and existence of prior negative prostate biopsy(-ies) demonstrate a higher positive predictive value for prostate cancer than the PSA test alone.

Levels (e.g., in ng/mL) of tPSA, fPSA, and hK2 present in human plasma samples are determined using the AutoDELFIA automatic immunoassay system. The averaged amount of each protein (marker) is calculated from the duplicate tests for each protein (marker) and used in a predictive model to determine a risk score for a given human plasma sample. tPSA and fPSA may also be determined using an Elecsys immunoassay analyzer (Roche Diagnostics).

Each run uses at least one plate. The whole procedure involves approximately 3 to 5 hours from the initiation to obtaining the test results depending on the number of plates being run.

### Example 7—Immunoassay Chip

Described herein is an immunoassay based on a panel of three kallikrein proteins (markers) that include total prostate specific antigen (tPSA), free PSA (fPSA), and human Kallikrein 2.

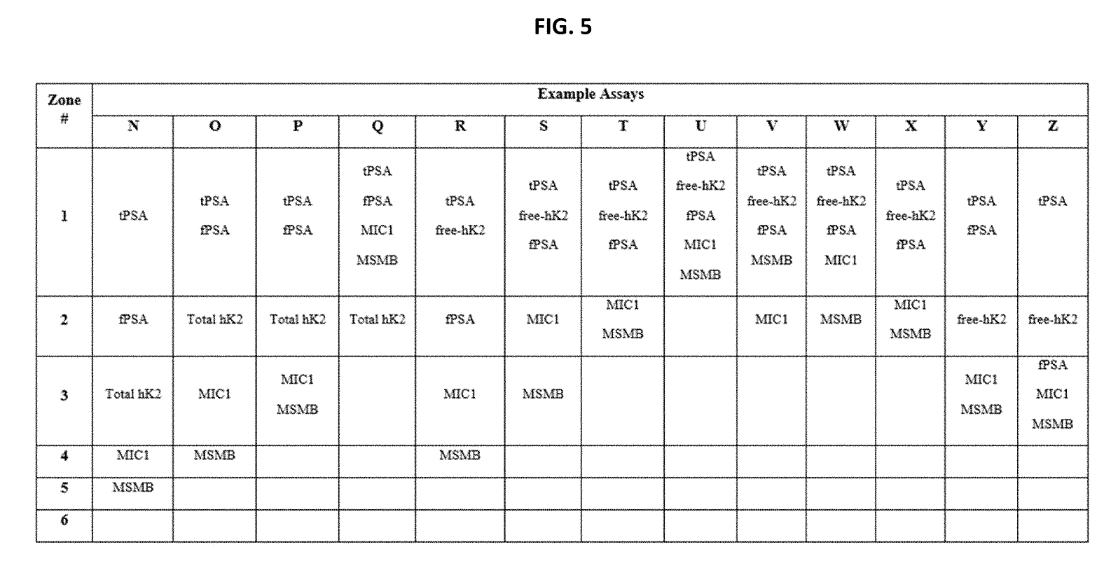

An immunoassay as described herein may be on a chip or slide that is made of a transparent material, such as glass. The immunoassay has one or more liquid containment regions. If the immunoassay has two or more liquid containment regions, these liquid containment regions are not in fluid communication (i.e., are fluidically isolated). Each liquid containment region has one or more analysis regions with binding proteins that recognize one or more kallikrein proteins, represented in FIGS. 1 and 2 as circles. Binding proteins for kallikrein proteins with common epitopes may be segregated into different liquid containment regions. Binding proteins for kallikrein proteins with unique epitopes may be in the same liquid containment regions. The different binding proteins may be spotted as replicates in a group (as shown in FIG. 1), or may be spotted in an interspersed manner (as shown in FIG. 2). As another example, FIG. 3 shows a chip that has 96 (6×12) liquid containment regions, with each liquid containment region defined as a fluidically independent zone with a group of spots. For example, one group of spots could be a grid of 60 analysis regions or spots. Each grid of spots could, for example, have binding partners for different proteins. The proteins (markers) that will bind to the binding partners present in liquid containment region should be selected so that they do not have common epitopes. The proteins (markers) that will bind to the binding partners present in each liquid containment region should be selected so that they have unique epitopes. As a specific example, one liquid containment region could have binding proteins for tPSA, fPSA, and free or total hK2; and a second liquid containment region could have binding proteins for MIC-1, and MSMB. In certain embodiments of the assay, binding partners for iPSA are absent from all liquid containment regions (i.e., no liquid containment regions have binding partners for iPSA). Exemplary assay combinations for use are shown in FIG. 4 and FIG. 5.

### Example 8—Exemplary Immunoassay A

Glass substrate (25×75 mm²) are thoroughly activated and cleaned with concentrated HCl, sequentially rinsed with deionized water, isopropanol, and high purity water (>18 MOhm cm) and dried with compressed nitrogen. The activated glass substrate are silanized in a 2% (v/v) of glycidoxypropyltrimethoxysilane in o-xylene (5 hours at 55° C.) with occasional mixing. The slides are rinsed with o-xylene, and dried at 135° C. A 2-mm thick Teflon mask with three 7×7 mm² openings is applied to the surface of the array, to prevent contact between reagents used in different openings. The capture probes (at a concentration of 2 mg/mL in phosphate buffer pH 8.4) are spotted with a commercial pin and ring instrument onto the surface of the glass, resulting in spots covering an area of approximately 0.2 mm in diameter. Three spots are prepared for each capture probe to generate triplicate measurements (for quality control procedure, see below). The capture probes for tPSA, fPSA, MIC1 and MSMB are spotted in a first opening and the capture probe for total hK2 is spotted in a second opening. A series of controls (purified human IgE at increasing concentrations from 1 to 300 ug/mL) is spotted in each opening. The spots are left to dry in ambient conditions, and after 1 hour are rinsed with a blocking solution (5% milk powder in TRIS buffer pH 7 with 0.2% Tween-20 and 0.05% sodium azide). Subsequently, the blocking solution is rinsed off with a stabilizer solution (PBS pH7.4 with 1% sucrose and 0.05% sodium azide), and then left to dry at ambient conditions for three hours.

Prior to use of the microarray for analysis of a sample, the surface of the opening is rinsed first with a rinse solution (150 mM sodium chloride, 10 mM Tris base, and 0.5% Tween 20, pH 8.0), then rinsed with deionized water, and finally dried in ambient conditions. An undiluted serum sample is pipetted in the first opening (20 μL). The serum is separately diluted 1:1 (v:v) with hK2 assay buffer (TRIS buffer pH 7.5 with BSA, tPSA antibodies, denatured mouse IgG, commercial HAMA blockers) and pipetted in the second opening. The samples are incubated for three hours in an environmental chamber (relative humidity >90% and temperature 25° C.). The surface of the microarray is then rinsed first with a rinse solution, then rinsed with deionized water, and finally dried at ambient conditions. The tracer antibodies solutions are then pipetted in the openings. The tracers for tPSA, fPSA, MIC1, and MSMB are pipetted in the first opening and the tracer for total hK2 mixed with tPSA blockers is pipetted in the third opening. Each tracer solutions also contains an anti-human IgE tracer. All tracer reagents are conjugates of the fluorescent dye Cy3. The tracer solutions are incubated for one hour in an environmental chamber (relative humidity >90% and temperature 25 degree C.), then rinsed with rinse solution, dried and stored in the dark.

The microarray is analyzed with a commercial laser scanner to image each spot and selected control areas between the spots (to evaluate non-specific binding, NSB). NSB must be below a pre-defined threshold in order for the measurements to be considered accurate. A quantitative fluorescence measurement is established for each spot by subtracting the NSB from the raw (unsubtracted) intensity of each spot. Consistency in the intensity across the triplicate measurements is assessed across the microarray. A maximum of one replicate is removed if a CV across triplicates is greater than 10%, and if outlier removal would bring the CV to less than 10%. The series of IgE controls is used to establish a relationship between fluorescent intensity and protein concentration, and the concentrations of tPSA, fPSA, MIC1, MSMB, and hK2 are calculated.

### Example 9—Exemplary Immunoassay B

Glass substrate (25×75 mm²) are thoroughly activated and cleaned with concentrated HCl, sequentially rinsed with deionized water, isopropanol, and high purity water (>18 MOhm cm) and dried with compressed nitrogen. The activated glass substrate are silanized in a 2% (v/v) of glycidoxypropyltrimethoxysilane in o-xylene (5 hours at 55° C.) with occasional mixing. The slides are rinsed with o-xylene, and dried at 135° C. A 2-mm thick Teflon mask with three 7×7 mm² openings is applied to the surface of the array, to prevent contact between reagents used in different openings. The capture probes (at a concentration of 2 mg/mL in phosphate buffer pH 8.4) are spotted with a commercial pin and ring instrument onto the surface of the glass, resulting in spots covering an area of approximately 0.2 mm in diameter. Three spots are prepared for each capture probe to generate triplicate measurements (for quality control procedure, see below). The capture probes for tPSA, hK2, and fPSA are spotted in a first opening; and the capture probes MIC1 and MSMB are spotted in the second opening. A series of controls (purified human IgE at increasing concentrations from 1 to 300 ug/mL) is spotted in each opening. The spots are left to dry in ambient conditions, and after 1 hour are rinsed with a blocking solution (5% milk powder in TRIS buffer pH 7 with 0.2% Tween-20 and 0.05% sodium azide). Subsequently, the blocking solution is rinsed off with a stabilizer solution (PBS pH7.4 with 1% sucrose and 0.05% sodium azide), and then left to dry at ambient conditions for three hours.

Prior to use of the microarray for analysis of a sample, the surface of the openings are rinsed first with a rinse solution (150 mM sodium chloride, 10 mM Tris base, and 0.5% Tween 20, pH 8.0), then rinsed with deionized water, and finally dried in ambient conditions. An undiluted serum sample is pipetted in the first opening and the second opening (20 μL). The serum is diluted 1:1 (v:v) with appropriate buffer solutions. The samples are incubated for three hours in an environmental chamber (relative humidity >90% and temperature 25° C.). The surface of the microarray is then rinsed first with a rinse solution, then rinsed with deionized water, and finally dried at ambient conditions. The tracer antibodies solutions are then pipetted in the openings. The tracers for tPSA, hK2, and fPSA are spotted in the first opening; and the tracers for MIC1 and MSMB are pipetted in the second opening. Each tracer solutions also contains an anti-human IgE tracer. All tracer reagents are labeled and may, for example, be conjugated to a fluorescent dye (for example, Cy3). The tracer solutions are incubated for one hour in an environmental chamber (relative humidity >90% and temperature 25 degree C.), then rinsed with rinse solution, dried and stored in the dark.

The microarray is analyzed with a commercial laser scanner to image each spot and selected control areas between the spots (to evaluate non-specific binding, NSB). NSB must be below a pre-defined threshold in order for the measurements to be considered accurate. A quantitative fluorescence measurement is established for each spot by subtracting the NSB from the raw (unsubtracted) intensity of each spot. Consistency in the intensity across the triplicate measurements is assessed across the microarray. A maximum of one replicate is removed if a CV across triplicates is greater than 10%, and if outlier removal would bring the CV to less than 10%. The series of IgE controls is used to establish a relationship between fluorescent intensity and protein concentration, and the concentrations of tPSA, fPSA, MIC1, MSMB, and hK2 are calculated.

### Example 10—Immunoassay and Model

Described herein is an assay based on a panel of three kallikrein proteins (markers) that include total prostate specific antigen (tPSA), free PSA (fPSA), and human Kallikrein 2 (total hK2 or free hK2). The three kallikrein proteins (markers) have been studied individually and in various combinations for prostate cancer detection applications.

Levels (e.g., in ng/mL) of tPSA, fPSA, free hK2 and/or total hK2 present in human plasma samples are determined. The levels of additional proteins (markers) such as MSMB and/or MIC-1 may also be determined using any method described herein. The averaged amount of each protein (marker) is calculated from the duplicate tests for each protein (marker) and subsequently analyzed using a predictive model to determine a risk score for a given human plasma sample. The level of any protein (marker) may also be determined using an analyzer such as an Elecsys immunoassay analyzer (Roche Diagnostics).

Each run uses at least one plate with two liquid containment regions, each of the liquid containment regions having one or more analysis region. The liquid containment regions may not be in fluidic communication (i.e., liquid cannot pass between the regions).

Additionally, genetic profile information may be gathered through, for example, levels of one or more SNPs. The SNPs may be any of the SNPs disclosed herein or known to be related to prostate cancer (including those SNPs generally known to be related to any proliferative disorder or risk factor for a proliferative disorder). Any number of SNPs can be chosen for this additional genetic analysis, and this selection of SNPs may be related to the population under examination. In certain embodiments, no SNPs are used and/or no additional genetic analysis is performed.

A logistic regression algorithm incorporating the blood plasma levels of recited proteins (markers) as well as subject-specific information such as age, results from a digital rectal exam (DRE), existence of prior negative prostate biopsy(-ies), and genetic profile information (such as that described above) may demonstrate a higher positive predictive value for prostate cancer than the PSA test alone.

1. A solid-phase assay system comprising:
a chip comprising a substantially rigid substrate and two or more fluidically isolated liquid containment regions,
each liquid containment region comprising one or more analysis regions,
wherein each analysis region comprises one or more binding partners immobilized to a substrate portion therein,
wherein the one or more binding partners in the first liquid containment region each specifically bind to an epitope present on one or more proteins selected from the group consisting of: total prostate specific antigen (tPSA), free prostate specific antigen (fPSA), total human kallikrein 2, and free human kallikrein 2 (fhK2); and
wherein the one or more binding partners in the second liquid containment region each specifically bind to an epitope present on one or more proteins selected from the group consisting of: intact prostate specific antigen (iPSA), macrophage inhibitory cytokine-1 (MIC-1), and beta-microseminoprotein (MSMB); and
a detector configured to measure a signal indicative of the extent of binding of the one or more binding partners to the one or more epitopes.

2-4. (canceled)

5. A solid-phase assay system comprising:
a chip comprising a substantially rigid substrate and one or more fluidically isolated liquid containment regions,
each liquid containment region comprising one or more analysis regions,
wherein each analysis region comprises one or more binding partners immobilized to a substrate portion therein,
wherein a first binding partner in the first liquid containment region binds to an epitope present on free prostate specific antigen (fPSA), total prostate specific antigen (tPSA), total human kallikrein 2 (thK2), and/or free human kallikrein 2 (fhK2); and
a detector configured to measure a signal indicative of the extent of binding of the one or more binding partners to the one or more epitopes.

6. (canceled)

7. The solid-phase assay system of claim 5, wherein the first liquid containment region or the second liquid containment region further comprises a binding partner that binds to an epitope present on a protein selected from the group: tPSA and fPSA; and wherein the first binding partner has about a 20-fold higher affinity for hK2 than for tPSA and/or fPSA.

8. The solid-phase assay system of claim 5, wherein the second liquid containment region further comprises a binding partner that binds to an epitope present on a protein selected from the group: tPSA and fPSA; and wherein the first binding partner and the second binding partner have approximately equal affinities for hK2.

9. The solid-phase assay system of claim 5, wherein the first liquid containment region or the second liquid containment region further comprises two or more binding partners that each bind to an epitope present on a protein selected from the group: MIC-1, MSMB, tPSA, and fPSA.

10. The solid-phase assay system of claim 5, wherein the first or second liquid containment region further comprises one or more binding partners that bind to an epitope present on intact prostate specific antigen (iPSA).

11. The solid-phase assay system of claim 5, wherein the solid-phase assay system further comprises one or more additional liquid containment regions.

12. The solid-phase assay system of claim 11, wherein the solid-phase assay system further comprises a third liquid containment region.

13. The solid-phase assay system of claim 12, wherein the third liquid containment region further comprises one or more binding partners that each bind to a protein selected from the group: MIC-1, MSMB, tPSA, and fPSA.

14. The solid-phase assay system of claim 12, wherein the third liquid containment region further comprises two or more binding partners that each bind to a protein selected from the group: MIC-1, MSMB, tPSA, and fPSA.

15. The solid-phase assay system of claim 12, wherein the third liquid containment region further comprises a binding partner that binds to MIC-1, and a binding partner that binds MSMB.

16. The solid-phase assay system of claim 12, wherein the third liquid containment region further comprises a binding partner that binds to iPSA.

17-20. (canceled)

21. A solid-phase assay system comprising:
a chip comprising a substantially rigid substrate and two or more fluidically isolated liquid containment regions, each liquid containment region comprising one or more analysis regions,
wherein each analysis region comprises one or more binding partners immobilized to a substrate portion therein, wherein a first binding partner in the first liquid containment region binds to an epitope present on a first kallikrein protein, wherein a second binding partner in the second liquid containment region binds to an epitope present on a second kallikrein protein,
wherein the first kallikrein protein and the second kallikrein protein have at least one common epitope; and
a detector configured to measure a signal indicative of the extent of binding of the one or more binding partners to the one or more epitopes.

22. The method of claim 21, wherein the first kallikrein protein and the second kallikrein protein are each selected from the group: free human kallikrein 2 (fhK2), total human kallikrein 2 (thK2), total prostate specific antigen (tPSA), and free prostate specific antigen (fPSA).

23. The method of claim 21, wherein the first kallikrein protein and the second kallikrein protein are each selected from the group: free human kallikrein 2 (fhK2), total human kallikrein 2 (thK2), total prostate specific antigen (tPSA), intact prostate specific antigen (iPSA), and free prostate specific antigen (fPSA).

24. The method of claim 21, wherein the first kallikrein protein is fPSA and the second kallikrein protein is iPSA.

25. The method of claim 21, wherein the first kallikrein protein is fPSA and the second kallikrein protein is fhK2 or thK2.

26. The method of claim 21, wherein the first kallikrein protein is fhK2 or thK2 and the second kallikrein protein is iPSA.

27. The method of claim 21, wherein the first kallikrein protein is tPSA and the second kallikrein protein is fhK2 or thK2.

28. An in vitro method, comprising:
a) introducing a sample from a subject into an assay system, wherein the assay system comprises the chip of any preceding claim;
b) allowing one or more epitopes from the sample to bind with the one or more binding partners in the one or more analysis regions;
c) determining a characteristic of one or more proteins using one or more detectors associated with the one or more analysis regions;
d) inputting the characteristics of the one or more proteins into a processor programmed to evaluate a logistic regression model to determine the probability of an event associated with prostate cancer in a subject, wherein evaluating the logistic regression model comprises scaling each of a plurality of variables by a different coefficient value to produce scaled variables and summing values for the scaled variables used to produce the probability of the event associated with prostate cancer in a subject, wherein the plurality of variables includes one or more clinical variables and two or more variables included in the information received from the detector; and
e) determining the probability of the event associated with prostate cancer in the subject.

29-35. (canceled)
